(19) United States
(12) Patent Application Publication
(10) Pub. No.: US 2024/0076609 A1
(43) Pub. Date: Mar. 7, 2024

(54) COMPOSITIONS AND METHODS FOR CULTURING AND EXPANDING CELLS

(71) Applicant: LIFE TECHNOLOGIES CORPORATION, Carlsbad, CA (US)

(72) Inventors: Angel VARELA-ROHENA, Frederick, MD (US); Jennifer DONATO, Williamsville, NY (US); Sarya MANSOUR, Grand Island, NY (US); Matthew DALLAS, Frederick, MD (US); Anna-Barbara HACHMANN, Buffalo, NY (US)

(21) Appl. No.: 18/176,123

(22) Filed: Feb. 28, 2023

Related U.S. Application Data
(63) Continuation of application No. 16/647,848, filed on Mar. 16, 2020, now abandoned.
(60) Provisional application No. 62/559,391, filed on Sep. 15, 2017.
(60) Provisional application No. 62/725,376, filed on Aug. 31, 2018.

(51) Int. Cl. C12N 5/00; C12N 5/0783

(52) U.S. Cl. CPC C12N 5/0037; C12N 5/0636; C12N 2500/34; C12N 2500/36; C12N 2510/02

(57) ABSTRACT

Provided herein are, inter alia, compositions, systems, kits, and methods for culturing and expanding cells (such as T cells, diploid or non-diploid cells), as well as methods for treating disorders (e.g., with T cells), and methods for producing biological molecules and compositions (e.g., proteins, viruses, viral particles or fragments thereof, etc.), including vaccines.

### BACKGROUND

Methods which allow for increased rate of cellular properties, such as cell division, have the potential for improving both bioproduction and therapeutic interventions, especially where cells are removed from an individual, cultured, and then reintroduced into that individual.

The ability of T cells to recognize the universe of antigens associated with, for example, various cancers or infectious organisms is conferred by T cell antigen receptor (TCR), which is made of both an α (alpha) chain and a β (beta) chain or a γ (gamma) and a 6 (delta) chain. T cells and various subsets have broad ranging therapeutic implications in the treatment of cancers, autoimmune disorders, inflammatory diseases, allergic diseases, and infectious diseases. There is a long felt need for reliable, efficient and rapid way to expand T cells, as well as other cells.

### BRIEF SUMMARY OF THE INVENTION

Provided are compositions and methods for culturing and/or expanding cells (e.g., human cells) where the cells produce one or more products (e.g., one or more protein (e.g., one or more heterologous protein), one or more nucleic acid molecule (e.g., one or more heterologous protein), one or more virus, and/or one or more VLP). Further provided herein are, inter alia, compositions, systems, kits, and methods for culturing and/or expanding cells (e.g., T cells), as well as methods for treating disorders with cells (e.g., T cells).

In an aspect, a culture medium composition (e.g., a serum-free cell culture medium composition) comprising a cyclodextrin and at least one lipid is provided herein. In some embodiments, the composition comprises linoleic acid, at least one other omega-6 fatty acid, cholesterol, and a methylated cyclodextrin.

In an aspect, a culture supplement composition (e.g., a serum-free cell culture supplement composition) comprising a cyclodextrin and at least one lipid is included herein. In some embodiments, the composition comprises linoleic acid, at least one other omega-6 fatty acid, cholesterol, and a methylated cyclodextrin.

Further provided herein are cell culture media compositions (e.g., serum-free cell culture media compositions) comprising linoleic acid, at least one other omega-6 fatty acid, cholesterol, and at least one cyclodextrin and cell culture supplement compositions (e.g., serum-free cell culture supplement compositions) comprising linoleic acid, at least one other omega-6 fatty acid, cholesterol, and at least one cyclodextrin. In many instances, the cyclodextrin is a methylated cyclodextrin. In many instances, cyclodextrin is present at a level from about 50 μM to about 200 μM. In many instances, the cholesterol is a synthetic cholesterol and the cholesterol is present at a concentration of from about 5 μM to about 30 μM. Additionally, in some instances, the at least one other omega-6 fatty acid is a polyunsaturated omega-6 fatty acid. In some instances, the at least one other omega-6 fatty acid is or includes arachidonic acid. In some instances, the polyunsaturated omega-6 fatty acid is one or more fatty acid selected from the group consisting of arachidonic acid, linoleic acid, linolenic acid, myristic acid, oleic acid, palmitic acid, palmitoleic acid, and stearic acid. In some instances, the effective dilution of the cell culture supplement compositions is from about 1:10 to about 1:5000.

Cell culture media compositions (e.g., serum-free cell culture medium compositions) provided herein, and/or cell culture media supplements (e.g., serum-free cell culture media supplements) provided herein composition are capable for use in the culturing cells that can produce a protein (e.g., a heterologous protein), a nucleic acid molecule (e.g., a heterologous protein), a vaccine, a virus, a viral particle, a viral protein or nucleic acid, or a viral fragment.

Cell that may be cultivated using cell culture media compositions provided herein include fungal cells (e.g., yeast cells, such as Saccharomyces cerevisiae, plant cells and animal cells (e.g., insect cells, such as sf9 cells, and mammalian cells, such as human cells, chicken cells, monkey cells, etc.). In some instances, the animal cells are bovine cells, canine cells, feline cells, insect cells, avian cells, primate cells or human cells. Further, cells cultivated as set out herein may be diploid cells.

In some instances, cell culture media compositions provided herein are used to cultivate cells, wherein the cells are selected from the group consisting of MRC-5, MRC-5 RCB, MRC-9, WI-38, 2BS, Walvax-2, IMR-90, IMR-91, KMB-17, HUT series cell, Chang liver, U937, MDCK, CD4-expressing T cell, CD8-expressing T cell, VERO and any clone of the preceding cells.

In some instances, cell culture media compositions provided herein further comprising 2-deoxy-D-glucose.

In some instances, cell culture media compositions provided herein may be used to increase the growth (e.g., the growth rate, as compared to cell culture media compositions which omit one or more components) of cells, the viable cell density of the cell, the viral titer of a virus produced by an infected cell, or a combination thereof.

In some instances, cells cultivated using cell culture media compositions provided herein are infected with a virus (e.g., an animal virus, a plant virus, a bacteriophage, etc.) or contain heterologous nucleic acid which encodes one or more expression product (e.g., one or more protein such as one or more cytokine, erythropoietin, antibody, etc.) for which production is desired. In some instances, such the viruses are one or more virus selected from the group consisting of Varicella zoster virus (VZV), Rubella, Measles, Mumps, Hepatitis A, Adenovirus, Poliomyelitis, Rotavirus, Smallpox, Chickenpox, Yellow fever, Papillomavirus, Ebola virus, HIV, Rabies or vesicular stomatitis virus (VSV), and Dengue virus. In some instances, viral particle produced using cells cultivated in cell culture media compositions provided herein are derived from a Parvoviridae family, Retroviridae family, Flaviviridae family or a bacteriophage.

Cell culture supplement compositions provided herein may be added to basal media to culture cells (e.g., diploid cells) capable of producing proteins, vaccines, viruses, viral particles, viral proteins or nucleic acids, and/or a viral fragments. In many instances, such cells are cultured under serum-free conditions.

Further provided herein are methods for culturing a cell population (e.g., a diploid cell population), comprising incubating the cell population in a cell culture medium comprising a cyclodextrin and at least one lipid. In some instances, such methods comprises vaccine producing cells (e.g., diploid cells), where the method comprising incubating the cell population in a serum-free, cell culture medium comprising: (i) a cyclodextrin (e.g., methylated cyclodextrin), linoleic acid, at least one other omega-6 fatty acid (e.g., one or more polyunsaturated omega-6 fatty acid, such as one or more of the following: arachidonic acid, linolenic acid, myristic acid, oleic acid, palmitic acid, palmitoleic acid, and stearic acid) and cholesterol, or, a suitable dilution of the supplements described in Table 1 and/or Table 2; wherein the culture increases viable cell density in said serum-free, cell culture medium compared to a viable cell density in a serum-containing medium without cyclodextrin.

In some instances, methods for culturing a cell population (e.g., a diploid cell population) include instances where: (i) the medium or supplement increases: the growth of the cell, the viable cell density of the cell, the viral titer of a virus infected cell, or a combination thereof, and/or, (ii) the cell (e.g., the diploid cell) is capable of producing a vaccine, a virus, a viral particle, a viral protein or nucleic acid, or a viral fragment thereof under serum-free conditions; and/or, (iii) said cell is infected with a virus. Further, in some instances: (i) the virus is an animal virus, a plant virus or a bacteriophage; and/or, (ii) the virus is selected from the group consisting of Varicella zoster virus (VZV), Rubella, Measles, Mumps, Hepatitis A, Adenovirus, Poliomyelitis, Rotavirus, Smallpox, Chickenpox, Yellow fever, Papillomavirus, Ebola virus, HIV, Rabies or vesicular stomatitis virus (VSV), and Dengue virus; and/or, (iii) the viral particle is derived from a Parvoviridae family, Retroviridae family, Flaviviridae family or a bacteriophage.

In some instances, the cell population cultivated using compositions and methods provided herein are selected from the group consisting of: MRC-5 cells, MRC-5 RCB cells, MRC-9 cells, WI-38 cells, 2BS cells, Walvax-2 cells, IMR-90 cells, IMR-91 cells, KMB-17 cells, HUT series cell, Chang liver cells, U937 cells, MDCK cells, CD4-expressing T cells, CD8-expressing T cells, VERO cells, and any clone of these cells.

Further provided herein are combinations comprising: (A) (i) a population of cells; (ii) a serum-free cell culture medium that comprises a cyclodextrin and at least one lipid, OR, (B) (i) a population of cells; (ii) a serum-free basal cell culture medium; and (iii) a supplement that comprises a cyclodextrin and at least one lipid, OR (C) (i) a population of cells; (ii) a serum-free basal cell culture medium; and (iii) a suitable dilution of the supplements described in Table 1 and/or Table 2. In some instances of such combinations (i) the cell is and animal cell; and/or, (ii) the animal cell is a bovine cell, a feline cell, an insect cell, an avian cell, a primate cell or a human cell; and/or, (iii) the animal cell is a diploid cell; and/or, (iv) the cell is selected from the group consisting of MRC-5 cells, MRC-5 RCB cells, MRC-9 cells, WI-38 cells, 2BS cells, Walvax-2 cells, IMR-90 cells, IMR-91 cells, KMB-17 cells, HUT series cell, Chang liver cells, U937 cells, MDCK cells, CD4-expressing T cells, CD8-expressing T cells, VERO cells, and any clone of these cells. In some instances the cells (e.g., diploids cells) produce a vaccine, a virus, a viral particle, a viral protein or nucleic acid, or a viral fragment under serum-free conditions.

Further provided herein are methods of making a cell culture medium (e.g., a serum-free culture medium, a serum-free diploid cell culture medium, etc.), comprising admixing (i) a basal medium; and either (ii) a supplement that comprises a cyclodextrin and at least one lipid; or (ii) a suitable dilution of the supplements described in Table 1 and/or Table 2. In some instances, the supplement further comprises one or more growth factors.

Further provided herein are systems for the supplementation of a cell medium (e.g., a diploid cell medium), comprising (i) a one or more different cyclodextrins, wherein each cyclodextrin is in a separate vessel; and (ii) two or more different fatty acids, wherein each fatty acid is in a separate vessel.

Further provided herein are kits for culturing a cell or cell line comprising: (i) a population of cells; (ii) a serum-free cell culture medium that comprises a cyclodextrin and at least one lipid, and/or, (i) a population of cells; (ii) a serum-free basal cell culture medium; and (iii) a supplement that comprises a cyclodextrin and at least one lipid, and/or, (i) a population of cells; (ii) a serum-free basal cell culture medium; and (iii) a suitable dilution of the supplements described in Table 1 and/or Table 2. Kits provided herein include those wherein: (i) the cell is animal cell; or, (ii) the animal cell is a bovine cell, a feline cell, an insect cell, an avian cell, a primate cell or a human cell; or, (iii) the animal cell is a diploid cell; or, (iv) the cell is selected from the group consisting of MRC-5 cells, MRC-5 RCB cells, MRC-9 cells, WI-38 cells, 2BS cells, Walvax-2 cells, IMR-90 cells, IMR-91 cells, KMB-17 cells, HUT series cell, Chang liver cells, U937 cells, MDCK cells, CD4-expressing T cells, CD8-expressing T cells, VERO cells and any clone of the preceding cells. Kits provided herein also include those wherein the cell produces a vaccine, a virus, a viral particle, a viral protein or nucleic acid, or a viral fragment under serum-free conditions.

In an aspect, provided herein are methods for culturing a cell population (e.g., a T cell population). Such methods include incubating the population in a cell culture medium comprising a cyclodextrin and at least one lipid.

In an aspect, provided herein are methods of culturing a T cell population that comprises CD8+ T cells and CD4+ T cells while minimizing a change in the ratio of CD8+ T cells to CD4+ T cells within the population. Such methods include incubating the population in a medium comprising a cyclodextrin and at least one lipid (e.g., a polyunsaturated fatty acid).

In an aspect, provided herein are methods for preferentially expanding members of a cell subpopulation (e.g., a T cell subpopulation). Such methods comprise exposing a mixed population of cells (e.g., T cells) to: (i) cyclodextrin; and (ii) fatty acids. In some embodiments, the molar ratio of two or more fatty acids is adjusted to induce the members of the cell subpopulation (e.g., a T cell subpopulation) to preferentially expand over members of other cell subpopulations (e.g., one of more other T cell subpopulations).

In an aspect, provided herein are methods of culturing a T cell population that comprises CD8+ T cells and CD4+ T cells while increasing the ratio of CD8+ T cells to CD4+ T cells within the population. Such methods comprise incubating the population in a cell culture medium comprising 2-deoxy-D-glucose (2-DG).

The invention further includes compositions and method for adjusting and/or maintaining ratio of CD8+ T cells to CD4+ T cells within the populations. Such methods include those where cells of a mixed population of CD8+ T cells to CD4+ T cells are contacted with (1) cyclodextrin/lipid compositions set out herein, (2) 2-DG, and/or combinations of (1) and (2). In some instances, it is desirable to generate or maintain populations of T cells where the ratio of CD8+ T cells to CD4+ T cells is at or near 1:1. In this a ratio context “near 1:1” refers to less than 10% variation. Thus, a ratio of 1:0.95 CD8+ T cells to CD4+ T cells would be near 1:1.

In some instances, it may be desirable to generate and/or maintain populations of T cells where the ratio of CD8+ T cells to CD4+ T cells is not at or near 1:1. In such instances, either CD8+ T cells or CD4+ T cells may predominate in the population. As an example, in some instances, it may be desirable to have a T cell population where the number of CD4+ T cells is two-fold higher than the number of CD8+ T cells (a 1:0.5 ratio of CD4+ T cells to CD8+ T cells). The invention thus provides compositions and methods for generating and/or maintaining T cell populations where the ratio of CD8+ T cells to CD4+ T cells or where the ratio of CD4+ T cells to CD8+ T cells is from about 1:1 to about 1:0.1 (e.g., from about 1:1 to about 1:0.2, from about 1:1 to about 1:0.3, from about 1:1 to about 1:0.4, from about 1:1 to about 1:0.5, from about 1:1 to about 1:0.7, etc.).

In an aspect, provided herein are methods for treating a disease in a subject in need thereof, comprising administering to the subject T cells obtained using a method, composition, kit, or system provided herein.

In an aspect, provided herein is a combination comprising (i) a population of cells (e.g., T cells), (ii) a cell culture medium that comprises a cyclodextrin and at least one lipid, and/or (iii) a cell culture medium that comprises 2-deoxy-D-glucose (2-DG).

In an aspect, provided herein is a biofermentor comprising the combination of a medium and/or supplement provided herein and a population of cells (e.g., T cells). In an aspect, provided herein is a cell culture plate or flask comprising the combination of a medium and/or supplement provided herein and a population of cells (e.g., T cells).

In an aspect, a system for the supplementation of a cell medium (e.g., a T cell medium) is provided. The system includes (i) two or more different cyclodextrins, wherein each cyclodextrin is in a separate vessel; (ii) two or more different fatty acids, wherein each fatty acid is in a separate vessel; and (iii) 2-deoxy-D-glucose (2-DG).

In an aspect, a kit for culturing cells (e.g., T cells) is provided. The kit may include one or more of the following: a culture medium (e.g., a serum free culture medium), a cyclodextrin, one or more lipids, and/or 2-deoxy-D-glucose (2-DG).

Also provided herein are combinations comprising (i) a population of cells (e.g., diploid or non-diploid cells) and (ii) a cell culture medium that comprises a cyclodextrin and at least one lipid. In some instances, the cell (e.g., the diploid cell) produces a vaccine, a virus, a viral particle, a viral protein or nucleic acid, or a viral fragment. In many instances, the combinations will be serum-free.

Further provided herein are methods for culturing a cell population (e.g., a diploid cell population), comprising incubating the cell population in a cell culture medium comprising a cyclodextrin and at least one lipid. In many instances, the cell culture medium will be serum-free. In some instances, the cell population is selected from the group consisting of MRC-5, MRC-5 RCB, WI-38, 2BS, Walvax-2, IMR-90, IMR-91, KMB-17, VERO cells, Chang liver cells, U937, MRC-9, MDCK, CD4-expressing T cells, CD8-expressing T cells, HUT (T cell line) series (for e.g. HUT78, HUT102, etc.) or clones of any such cell.

In some instances, the cell produces a vaccine, a virus, a viral particle, a viral protein or nucleic acid, or a viral fragment. In many instances, such production will be under serum-free conditions.

Also provided herein are systems for the supplementation of a cell medium (e.g., a diploid cell medium), comprising (i) a one or more different cyclodextrins, wherein each cyclodextrin is in a separate vessel; and (ii) two or more different fatty acids, wherein each fatty acid is in a separate vessel. Such systems may further comprise growth factors.

Further provided herein are systems for the supplementation of a diploid cell medium, comprising (i) a cyclodextrin and (ii) two or more different fatty acids, wherein each fatty acid is in a separate vessel.

Also provided are kits for culturing a vaccine producing cell or cell line comprising a basal medium and a serum free growth supplement. In some instances, the serum free growth supplement in such kits will comprise a cyclodextrin and one or more lipid. Further, the kit may contain or be suitable for culturing a vaccine producing cell or cell line. In some instances, the vaccine producing cell is a diploid cell (e.g., a human cell) or a non-diploid cell.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the data presented in Table 4. Results indicate that CD Supplements 1, 2, and 3 (1:500) increase T cell expansion expressed as cumulative population doublings over time compared to Lipid Concentrate (1:100) and medium without lipid supplementation. Titrations of CD Supplements 1, 2, and 3 indicate that high concentrations (1:250) cause toxicity, while low concentrations (1:1000, 1:1250) yield fewer population doublings, and therefore decreased T cell expansion.

FIG. 2 depicts the data presented in Table 5, highlighting selected conditions from the expansion in FIG. 1. Results demonstrate CD Supplements 1, 2, and 3 (1:500) yield more population doublings than Lipid Concentrate and medium without lipid supplementation.

FIG. 3 depicts the data presented in Table 6, which is the same growth data in FIG. 1 (Table 4) but represented in cumulative viable T cells overtime. Results demonstrate CD Supplements 1, 2, and 3 (1:500) yield the most viable T cells by day 12. Titrations of CD Supplements 1, 2, and 3 indicate that high concentrations (1:250) cause toxicity, while low concentrations (1:1000, 1:1250) yield fewer viable T cells, and therefore decreased T cell expansion. The difference in performance among CD Supplements 1, 2, and 3 become more evident when represented in cumulative cell number compared to cumulative population doublings over time.

FIG. 4 depicts the data presented in Table 7, highlighting selected conditions from the expansion in FIG. 3. Results demonstrate CD Supplements 1, 2, and 3 (1:500) yield more cumulative viable T cells than Lipid Concentrate and medium without lipid supplementation.

FIG. 5 depicts the data presented in Table 8, which represents T cell viability during the expansion represented by FIGS. 1 and 3. Results demonstrate CD Supplements 1, 2, and 3 (1:500) maintain increased cell viability throughout expansion compared to other CD Supplement concentrations, Lipid Concentrate, and no lipid supplementation. Titrations of CD Supplements 1, 2, and 3 indicate that high concentrations (1:250) cause toxicity, while low concentrations (1:1000, 1:1250) may not provide enough lipids to maintain increased cell viability.

FIG. 6 depicts the data presented in Table 9, highlighting selected conditions from the expansion in FIG. 5. Results demonstrate CD Supplements 1, 2, and 3 (1:500) maintain increased cell viability throughout expansion compared to Lipid Concentrate and medium without lipid supplementation.

FIG. 7 depicts the gating strategy for differentiation phenotyping. T cells expanded for 10 days were stained with antibodies against CD3, CD4, CD8, CCR7, and CD62L. Sequential gating was used to characterize T cells as central memory (TCM: CCR7+/CD62L+), intermediate (CCR7−/CD62L+), and effector memory (TEM: CCR7−/CD62L−). Flow cytometric analysis was performed in a Beckman-Coulter Gallios analyzer.

FIG. 8 depicts the average percentage of CD4+ and CD8+ T cells in the gated CD3+ population, presented in Table 10. Day 0 represents the average frequency of the two populations prior to expansion. Results demonstrate a preferential expansion of CD8+ T cells in culture medium supplemented with CD Sup. 1 compared to medium supplemented with 5% human AB serum and CD Supplements 2 and 3. Error bars represent the standard deviation among three replicates. Without being bound by any theory, since CD Supp. 1 contains all polyunsaturated fatty acids, and yields the most favorable CD4+/CD8+ ratio, one can expect that polyunsaturated fatty acids yield more CD8+ cell growth. Specifically, since CD Supp. 1 contains mainly Omega-6 polyunsaturated fatty acids and essentially fatty acids, these specific fatty acids may be contributing to the increased CD8+ cell expansion.

FIG. 9 depicts the differentiation status of CD4+ T cells expanded in 5% human AB serum and CD Supplements 1, 2, and 3. CD4+ T cells cultured with 5% human AB serum lose the CCR7+/CD62L+ phenotype and accumulate the CCR7−/CD62L-phenotype, indicating cellular stress and nutritional deficiencies. Alternatively, CD4+ T cells cultured with CD Supplements 1, 2, and 3 avoid CCR7−/CD62L-accumulation.

FIG. 10 depicts the differentiation status of CD8+ T cells expanded in 5% human AB serum and CD Supplements 1, 2, and 3. CD8+ T cells cultured with 5% human AB serum lose the CCR7+/CD62L+ phenotype and accumulate the CCR7−/CD62L-phenotype, indicating cellular stress and nutritional deficiencies. Alternatively, CD8+ T cells cultured with CD Supplements 1, 2, and 3 avoid CCR7−/CD62L-accumulation.

FIG. 11 compares Th1 cytokine profiles between T cells grown in medium containing either 5% human AB serum or CD Sup. 1. Primary human T cells from normal donors were negatively isolated from PBMCs with DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell and cultured in serum-free medium supplemented with CD Sup. 1 (1:500) or 5% human AB serum. T cells were counted and fed on days 5 and 7 on a Beckman-Coulter Vi-Cell analyzer and fed to a density of 5×10⁵ vc/mL on days 3, 5, and 7. DYNABEADS® were removed from the cultures on day 11 and cells were spun to remove conditioned medium and rested overnight in fresh medium. One million T cells were re-stimulated with DYNABEADS® CD3 at a 1:1 bead to cell ratio and incubated for 24 hours. Supernatants were collected and processed for analysis with Invitrogen Cytokine Human Magnetic 35-Plex Panel for LUMINEX™. As depicted in FIG. 11, results demonstrate that the cytokine profile of T cells cultured in serum-free medium plus CD Sup. 1 is comparable, if not slightly better, than the profile of T cells cultured with 5% human AB serum. This is represented by the increase in MIP-1Alpha, decrease in IL-13, IL-10, and IL-6, and no change in IFN-γ and IL-2 production (Table 11). Viable Cells Per Milliliter=vc/mL.

FIG. 12 shows data (Table 12) obtained from the titration of 2-DG with Pan CD3+ T cells. X-VIVO™ 15 medium supplemented with 5% human AB serum was used as a positive control. Briefly, 2-DG was prepared in sterile filtered water at a stock concentration of 100 mM. Primary human T cells from normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell and cultured in serum-free medium free of cholesterol and free fatty acids supplemented with one of four lipid supplements. T cells were counted on days 5, 7, 10, and 12 on a Beckman-Coulter Vi-CELL XR analyzer and fed to a density of 5×10⁵ vc/mL on days 3, 5, and 7 and 1×10⁶ vc/mL on day 10. FIG. 12 results demonstrate expansion comparable to control in cultures treated with 2-DG at 0.25 mM and 0.5 mM concentrations.

FIGS. 13 and 14 represent the data shown in Table 13 which depicts the gating strategy for differentiation phenotyping. T cells expanded for 10 days were stained with antibodies against CD3, CD4, and CD8. Flow cytometric analysis was performed in a Beckman-Coulter Gallios analyzer. Results show that supplementing cells with either 0.25 mM 2-DG or 0.5 mM 2-DG result in about a 3-fold increase in CD8+ to CD4+ ratio.

FIGS. 15 and 16 represent the growth curves of cultured naïve and non-naïve T cells with 4 mM 2-DG. The data is represented in Tables 14 and 15. CD8+ and CD4+ T cells were isolated from PBMCs by negative selection using Untouched Human CD8+ and CD4+ T Cells Kits. Naïve and non-naïve T cells were isolated from enriched T cells by positive selection using CD45RA nanobeads (Miltenyi). Naïve and non-naïve T cells from Pan CD3+ T cells were purified is to determine if 2-DG has an effect on terminally or non-terminally differentiated donors (non-naïve T cells). Both cell types grew with 2-DG.

FIGS. 17 and 18 depict the data shown in Tables 16 and 17, which illustrates the gating strategy for differentiation phenotyping. T cells expanded for 10 days were stained with antibodies against CD3, CD4, and CD8. Results highlight that 2-DG has a much stronger effect on naïve T cells (3.2 fold increase in CD8+ to CD4+ T cell ratio) than on non-naïve T cells.

FIGS. 19 and 20 represent the growth curves of 2-DG in mixed CD4+ T cells and CD8+ T cells at set ratio (5:1 and 10:1 respectively). Both ratios of cells grew similarly. (See Tables 18 and 19.)

FIGS. 21 and 22 depict the average percentage of CD4+ and CD8+ T cells in the gated CD3+ population, presented in Tables 20 and 21. Day 0 represents the average frequency of the two populations prior to expansion. These results demonstrate that 2-DH is able to correct for substantial deficits in CD8+ T cells compared to day 0.

FIG. 23 illustrates protocols used for the culturing of T cells for 12 days. Stimulation of T cells with DYNABEADS® Human T-Expander CD3/CD28 at a ratio of 3 beads per T cell and cultured in serum-free, animal origin free medium and culturing the cells with 2-DG. Followed by feeding the cells with 2-DG at various time points (day 3, day 5, day 7, and every time cells were fed).

FIGS. 24 and 25 show the growth curves of T cells cultured in presence or absence of 2-DG as well as introducing it during expansion at different time points as indicated. Treatment with supplement was started on day 0, 3, 5, 7 and maintained with subsequent feedings. T cells were cultured for 12 days in serum-free T cell medium and in X-VIVO™ 15 medium supplemented with 5% human AB serum.

FIG. 26 represents the data shown in Table 24 which highlights the fold increase in CD8+ to CD4+ ratio of T cells cultured in absence or presence of 2-DG at different time points and with subsequent feedings. Results demonstrate that culturing T cells with 0.25 mM 2-DG on day 7 only and every time cells were fed resulted in the same 3-fold increase in CD8+ T cells.

FIG. 27 represents the expansion T cells for 12 days and re-stimulated with DYNABEADS® Human T-Expander CD3/CD28. Cytokine production upon re-stimulation was assessed with Invitrogen Cytokine Human Magnetic 35-Plex Panel for LUMINEX™. Fifteen cytokines shown out of 35-plex assay. All values were normalized relative to X-VIVO™ 15 medium. Results demonstrate that 2-DG does not alter the function of the cells as measured by multiplexed cytokine assay.

FIG. 28 describes the titration of 2-Deoxy-D-Glucose (2-DG) (0, 1 mM, 2 mM, and 4 mM) in Pan CD3+ T cells (Table 25). X-VIVO™ supplemented with 5% human AB serum was added as a positive control. Briefly, 2-DG was prepared in sterile filtered water at a stock concentration of 100 mM. Primary human T cells from normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell and cultured in serum-free medium free of cholesterol and free fatty acids supplemented with one of four lipid supplements. T cells were counted on days 5, 7, 10, and 12 on a Beckman-Coulter Vi-CELL XR analyzer and fed to a density of 5×10⁵ vc/mL on days 3, 5, and 7 and 1×10⁶ vc/mL on day 10. Results show that supplementation with 2-DG does not affect cell growth.

FIGS. 29 and 30 represent the data shown in Table 26 which depicts the gating strategy for differentiation phenotyping. T cells expanded for 10 days were stained with antibodies against CD3, CD4, and CD8. Flow cytometric analysis was performed in a Beckman-Coulter GALLIOS™ analyzer. Results show that supplementing cells with 4 mM 2-DG result in about a 4.1 fold increase in CD8+ to CD4+ ratio.

FIGS. 31 and 32 represent the growth curves of cultured naïve and non-naïve T cells with 4 mM 2-DG. The data is represented in Tables 27 and 28. CD8+ and CD4+ T cells were isolated from PBMCs by negative selection using Untouched Human CD8+ and CD4+ T Cells Kits. Naïve and non-naïve T cells were isolated from enriched T cells by positive selection using CD45RA nanobeads (Miltenyi). The reason for purifying naïve and non-naïve T cells from Pan CD3+ T cells is to see if 2-DG will have any effect on terminally or non-terminally differentiated donors (non-naïve T cells). Both cell types grew with 2-DG.

FIGS. 33 and 34 depict the data shown in Tables 29 and 30 which illustrates the gating strategy for differentiation phenotyping. T cells expanded for 10 days were stained with antibodies against CD3, CD4, and CD8. Results highlight that 2-DG has a much stronger effect in naïve T cells (2.4 fold increase in CD8 to CD4 ratio) than in non-naïve T cells.

FIG. 35 represents data shown in Table 31 which describes the titration of 2-Deoxy-D-Glucose (2-DG) (0 mM, 0.25 mM, and 0.5 mM) in Pan CD3+ T cells. X-VIVO™ supplemented with 5% human AB serum was added as a positive control. Primary human T cells from normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell and cultured in serum-free medium free of cholesterol and free fatty acids supplemented with one of four lipid supplements. T cells were counted on days 5, 7, 10, and 12 on a Beckman-Coulter Vi-CELL XR analyzer and fed to a density of 5×10⁵ vc/mL on days 3, 5, and 7 and 1×10⁶ vc/mL on day 10. Results show that supplementation with 2-DG does not affect cell growth.

FIGS. 36 and 37 represent the data shown in Table 32 which depicts the gating strategy for differentiation phenotyping. T cells expanded for 10 days were stained with antibodies against CD3, CD4, and CD8. Flow cytometric analysis was performed in a Beckman-Coulter Gallios analyzer. Results show that 0.25 mM 2-DG and 0.5 mM 2-DG both result in about a 3-fold increase in CD8+ to CD4+ ratio. FIG. 36 data is a representative from a single donor. FIG. 37 is representative from 3 different donors.

FIGS. 38 and 39 signifies data shown in Tables 33 and 34 which represent the growth curves of 2-DG in mixed CD4+ T cells and CD8+ T cells at set ratio (5:1 and 10:1 respectively). Both ratios of cells grew similarly.

FIGS. 40 and 41 depict the average percentage of CD4+ and CD8+ T cells in the gated CD3+ population, presented in Tables 35 and 36. Day 0 represents the average frequency of the two populations prior to expansion. Results demonstrate that 2-DG is able to correct for a large deficits in CD8+ T cells compared to day 0.

FIG. 42 illustrates culturing of T cells for 12 days. Stimulation of T cells with DYNABEADS® Human T-Expander CD3/CD28 at a ratio of 3 beads per T cell and cultured in serum-free, animal origin free medium and culturing the cells with 2-DG. Followed by feeding the cells with 2-DG at various time points (day 3, day 5, day 7, and every time cells were fed).

FIGS. 43 and 44 show the growth curves of T cells cultured with 2-DG at different time points. 2-DG was cultured with the cells on day 0, 3, 5, 7 and maintained with subsequent feedings. T cells were cultured for 12 days in serum-free T cell medium and in medium with 5% Human AB Serum. Tables 37 and 38.

FIG. 45 represents the data shown in Table 39 which highlights the fold increase in CD8+ to CD4+ ratio of T cells cultured with 2-DG at different time points and every time the cells were fed. Results demonstrate that culturing T cells with 0.25 mM 2-DG on day 7 alone and at every time cells were fed resulted in the same 3-fold increase in CD8:CD4 ratio.

FIGS. 46 and 47 represent the growth curves of 2-DG cultured with different concentrations of CD Lipid Concentrate 1 and 2 respectively and in 5% Human AB Serum as a positive control shown in Tables 40 and 41. Cells were expanded for 12 days. Results demonstrate that 1:1000 CLC1 and CLC2 with 2-DG demonstrate the optimal cell expansion.

FIGS. 48 and 49 represent the data from Tables 42 and 43 which depicts the gating strategy for differentiation phenotyping. T cells expanded for 10 days were stained with antibodies against CD3, CD4, and CD8. Flow cytometric analysis was performed in a Beckman-Coulter Gallios analyzer. Results show that there is a 1.6 fold increase in CD8:CD4 ratio when adding 2-DG with CLC1 and a 1.4 fold increase in CD8:CD4 ratio when adding 2-DG with CLC2 compared to day 0, pre-expansion.

FIG. 50: Diploid viable cell density (VCD) is expressed as viable cells per milliliter. Results in this figure indicate that Diploid SFM yields cell growth in various diploid cell lines that are commonly used in vaccine production, e.g., MRC-5, WI-38, IMR-90, etc. Results are shown here for an exemplary diploid cell, e.g., MRC-5, and are representative of at least 3 independent experiments.

FIG. 51: Results in this figure indicate that CD Supplements 1 and 2 increase MRC-5 VCD compared to Lipid Concentrate (1:100 and 1:1000). Additionally, CD Supplements 1 and 2 yield comparable VCD to serum-containing medium at 1:500 and 1:2000 concentrations, respectively.

FIG. 52: Results in this figure indicate that MRC-5 cells grown in Diploid Growth SFM yield varicella zoster virus production comparable to that of cells grown in serum-containing medium.

### DETAILED DESCRIPTION OF THE INVENTION

### Definitions

Unless specifically defined otherwise, all technical and scientific terms used herein shall be taken to have the same meaning as commonly understood by one of ordinary skill in the art (e.g., in cell culture, immunology, molecular genetics, and biochemistry).

The following definitions are included for the purpose of understanding the present subject matter and for constructing the appended patent claims. Abbreviations used herein have their conventional meaning within the chemical and biological arts.

As used herein, the singular forms “a,” “an,” and “the” include the plural reference unless the context clearly dictates otherwise. Thus, for example, a reference to “a cyclodextrin” “a lipid” or “a fatty acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. It will be appreciated that, although not precluded, treating a disorder or condition does not require that the disorder, condition or symptoms associated therewith be completely eliminated. As used herein, “inhibition” of disease progression or a disease complication in a subject means preventing or reducing the disease progression and/or disease complication in the subject. As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention.

As used herein, the term “about” in the context of a numerical value or range means±10% of the numerical value or range recited or claimed, unless the context requires a more limited range.

In the descriptions herein, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg, etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, the term “lipid” includes waxes, fats, oils, fatty acids, sterols, monoglycerides, diglycerides, triglycerides, phospholipids, and others. In embodiments, a lipid is a substance such as a wax, fat, oil, fatty acid, sterol, monoglyceride, diglyceride, triglyceride, or phospholipid that dissolves in alcohol but not in water. In embodiments, a lipid is a fatty acid, a glycerolipid, a glycerophospholipid, a sphingolipid, a prenol lipid, a saccharolipid, or a polyketide. In embodiments, a lipid comprises a ketoacyl or an isoprene group. In embodiments, a lipid is a wax ester. In embodiments, a lipid is an eicosanoid (e.g., a prostaglandin, a thromboxane, a leukotriene, a lipoxins, a resolvin, or an eoxin). In embodiments, a lipid is a sterol lipid. In embodiments, the sterol lipid is cholesterol or a derivative thereof. In embodiments, the cholesterol is nat-cholesterol and/or ent-cholesterol.

As used herein, the term “fatty acid” refers to a carboxylic acid (or organic acid), often with a long aliphatic tail, either saturated or unsaturated. In embodiments, a fatty acid has a carbon-carbon bonded chain of at least 4 carbon atoms in length. In embodiments, a fatty acid has a carbon-carbon bonded chain of at least 8 carbon atoms in length. In embodiments, a fatty acid has a carbon-carbon bonded chain of at least 12 carbon atoms in length. In embodiments, a fatty acid has a carbon-carbon bonded chain of at between 4 and 24 carbon atoms in length. In embodiments, a fatty acid is a naturally occurring fatty acid.

In embodiments, a fatty acid is artificial (e.g., is not produced in nature). In embodiments, a naturally occurring fatty acid has an even number of carbon atoms. In embodiments, the biosynthesis of a naturally occurring fatty acid involves acetate which has two carbon atoms. In embodiments, a fatty acid may be in a free state (non-esterified) or in an esterified form such as part of a triglyceride, diacylglyceride, monoacyglyceride, acyl-CoA (thio-ester) bound orotherbound form. In embodiments, the fatty acid may be esterified as a phospholipid such as a phosphatidylcholine, phosphatidylethanolamine, phosphatidylserine, phosphatidyCglycerol, phosphatidylinositol or diphosphatidylglycerol form. In embodiments, a fatty acid or derivative of a fatty acid is a free fatty acid, an ester (e.g., methyl, ethyl, propyl, etc.), a mono-, di-, or triglyceride (e.g., a glycerol ester), an aldehyde, an amide, or a phospholipid version of a fatty acid disclosed herein. A “saturated fatty acid” does not contain any double bonds or other functional groups along the chain. The term “saturated” refers to hydrogen, in that all carbons (apart from the carboxylic acid [—COOH] group) contain as many hydrogens as possible. In other words, the omega end contains 3 hydrogens (CH3-) and each carbon within the chain contains 2 hydrogens (—CH2-). In an “unsaturated fatty acid,” one or more alkene functional groups exist along the chain, with each alkene substituting a singly-bonded “—CH2-CH2-” part of the chain with a doubly-bonded “—CH═CH—” portion (that is, a carbon double bonded to another carbon). The two next carbon atoms in the chain that are bound to either side of the double bond can occur in a cis or trans configuration. A table of non-limiting examples of fatty acids is as follows:

| | | | Omega-3, | Essential |
|---|---|---|---|---|
| Saturation | Lipid Number | Common Name | 6 or 9? | Fatty Acid? |
| Saturated | 4:0 | Butyric acid | | |
| | 8:0 | Caprylic acid | | |
| | 10:0 | Capric acid | | |
| | 12:0 | Lauric acid | | |
| | 14:0 | Myristic acid | | |
| | 16:0 | Palmitic acid (PA) | | |
| | 18:0 | Stearic acid (SA) | | |
| | 20:0 | Arachidic acid | | |
| | 22:0 | Behenic acid | | |
| | 24:0 | Lignoceric acid | | |
| | 26:0 | Cerotic acid | | |
| Monounsaturated | 16:1 | Palmitoleic Acid | | |
| | 18:1(n-9) | Oleic acid (OA) | Omega-9 | |
| | 20:1(n-9) | Eicosenoic acid | Omega-9 | |
| | 22:1(n-9) | Erucic acid | Omega-9 | |
| | 24:1(n-9) | Nervonic acid | Omega-9 | |
| Polyunsaturated | 16:3(n-3) | Hexadecatrienoic acid | Omega-3 | |
| | | (HTA) | | |
| | **18:2(n-6) | Linoleic acid (LA) | Omega-6 | Yes |
| | **18:3 (n-3) | Alpha-linolenic acid | Omega-3 | Yes |
| | | (ALA) | | |
| | **18:3 (n-6) | Gamma-linolenic acid | Omega-6 | |
| | | (GLA) | | |
| | 18:4(n-3) | Stearidonic acid (SDA) | Omega-3 | |
| | 20:2(n-6) | Eicosadienoic acid | Omega-6 | |
| | 20:3(n-3) | Eicosatrienoic acid (ETE) | Omega-3 | |
| | 20:3(n-6) | Dihomo-gamma-linolenic | Omega-6 | |
| | | acid (DGLA) | | |
| | 20:3(n-9) | Mead acid | Omega-9 | |
| | **20:4(n-6) | Arachidonic acid (AA) | Omega-6 | |
| | 20:4(n-3) | Eicosatetraenoic acid | Omega-3 | |
| | | (ETA) | | |
| | 20:5(n-3) | Eicosapentaenoic acid | Omega-3 | |
| | | (EPA, Timnodonic acid) | | |
| | 21:5(n-3) | Heneicosapentaenoic acid | Omega-3 | |
| | | (HPA) | | |
| | 22:2(n-6) | Docosadienoic acid | Omega-6 | |
| | 22:4(n-6) | Adrenic acid | Omega-6 | |
| | 22:5(n-3) | Docosapentaenoic acid | Omega-3 | |
| | | (DPA, Clupanodonic acid) | | |
| | 22:5(n-6) | Docosapentaenoic acid | Omega-6 | |
| | | (Osbond acid) | | |
| | 22:6(n-3) | Docosahexaenoic acid | Omega-3 | |
| | | (DHA, Cervonic acid) | | |
| | 24:4(n-6) | Tetracosatetraenoic acid | Omega-6 | |
| | 24:5(n-3) | Tetracosapentaenoic acid | Omega-3 | |
| | 24:5(n-6) | Tetracosapentaenoic acid | Omega-6 | |
| | 24:6(n-3) | Tetracosahexaenoic acid | Omega-3 | |
| | | (Nisinic acid) | | |

Underlined fatty acids indicate fatty acids tested in CD Supplements 1, 2, and 3.
**indicates fatty acids that are in CD Supp. 1 of Examples 1-4. CD Supp. 1, 2, and 3 of Examples 1-4 contain a 50/50 mix of alpha- and gamma-linolenic acids.

Cyclodextrins (sometimes called cycloamyloses) are compounds made up of sugar molecules bound together in a ring (cyclic oligosaccharides). In embodiments, a cyclodextrin comprises of 5 or more α-D-glucopyranoside units linked 1-4 [e.g., an α(1-4) linkage]. In embodiments, a cyclodextrin is a cyclomalto-oligosaccharide having at least five glucopyranose units Joined by an α(1-4) linkage. In embodiments, cyclodextrins are cyclic oligosaccharides with hydroxyl groups on the outer surface and a void cavity in the center. In embodiments, cyclodextrins are characterized by a rigid, truncated conical molecular structure having a hollow interior, or pore (e.g., cavity), of specific volume. Cyclodextrins are capable of forming inclusion complexes with a wide variety of hydrophobic molecules by taking up a whole molecule, or some part of it, into the center (e.g., cavity) thereof. In embodiments, the stability of the complex formed depends on how well the guest molecule fits into the cyclodextrin cavity. Non-limiting examples of cyclodextrins include α-, β-, or γ-cyclodextrin wherein α-cyclodextrin has six glucose residues; β-cyclodextrin has seven glucose residues, and β-cyclodextrin has eight glucose residues. Cyclodextrins include cyclodextrin derivatives (e.g., derivatives of α-, β-, and γ-cyclodextrin), or a blend of one or more cyclodextrins and/or cyclodextrin derivatives. Common cyclodextrin derivatives are formed by alkylation (e.g. methyl- and ethyl-β-cyclodextrin) or hydroxyalkylation of the hydroxyl groups (e.g. hydroxypropyl- and hydroxyethyl-derivatives of α-, β-, and γ-cyclodextrin) or by substituting the primary hydroxyl groups with saccharides (e.g. glucosyl- and maltosyl-β-cyclodextrin).

In embodiments, a cyclodextrin comprises 6-8 glucopyranoside units, and can be topologically represented as a toroid with the larger and the smaller openings of the toroid exposing to the solvent secondary and primary hydroxyl groups respectively. In embodiments, the interior of the toroids is not hydrophobic, but considerably less hydrophilic than the aqueous environment and thus able to host other hydrophobic molecules. In contrast, the exterior is sufficiently hydrophilic to impart cyclodextrins (or their complexes) solubility in aqueous solutions.

As used herein, the term “monounsaturated fatty acid” refers to a fatty acid that comprises only one alkene group (carbon-carbon double bond) in the chain. As used herein, the terms “polyunsaturated fatty acid” and “PUFA” refer to a fatty acid which comprises at least two alkene groups (carbon-carbon double bonds).

As used herein, the terms “long-chain polyunsaturated fatty acid” and “LC-PUFA” refer to a fatty acid which comprises at least 20 carbon atoms in its carbon chain and at least two carbon-carbon double bonds, and hence include VLC-PUFAs. As used herein, the terms “very long-chain polyunsaturated fatty acid” and “VLC-PUFA” refer to a fatty acid which comprises at least 22 carbon atoms in its carbon chain and at least two or three carbon-carbon double bonds. Ordinarily, the number of carbon atoms in the carbon chain of a fatty acid refers to an unbranched carbon chain. If the carbon chain is branched, the number of carbon atoms excludes those in side-groups.

In embodiments, a fatty acid is an omega-3 fatty acid having a desaturation (carbon-carbon double bond) in the third carbon-carbon bond from the methyl end of the fatty acid. In embodiments, a fatty acid is an omega-6 fatty acid having a desaturation (carbon-carbon double bond) in the sixth carbon-carbon bond from the methyl end of the fatty acid. In embodiments, a fatty acid is an omega-9 fatty acid having a desaturation (carbon-carbon double bond) in the ninth carbon-carbon bond from the methyl end of the fatty acid.

As used herein, the term “heterologous”, when used with respect to a cell, refers to a material (e.g., a protein, a nucleic acid, a protein or protein nucleic acid complex, a virus, etc.) that is not normally associated with the cell. For purposes of clarity, a virus which naturally infects a cell would be considered to be “heterologous” to that cell because the virus not a component which is normally associated with the cell in the cell's natural state. Thus, expression vectors introduced into a cell would also be considered to be “heterologous”.

The term “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. In embodiments, the methods disclosed herein are suitable for use in a patient that is, e.g., a member of the Vertebrate class, Mammalia, including, without limitation, primates, rodents, livestock, and domestic pets (e.g., a companion animal). Typically, a patient will be a human patient.

The terms “subject,” “patient,” “individual,” and the like as used herein are not intended to be limiting and can be generally interchanged. That is, an individual described as a “patient” does not necessarily have a given disease, but may be merely seeking medical advice.

The term “subject” as used herein includes all members of the animal kingdom that may suffer from the indicated disorder. In embodiments, the subject is a mammal. In embodiments, the subject is a primate, a non-primate, or a rodent. In embodiments, the subject is a human. In embodiments, the subject is a research animal. In embodiments, the subject is a work animal (e.g., a police or military dog or horse), a service animal, or a domestic pet. In embodiments, the subject is a dog, cat, horse, cow, pig, mouse, rat, camel, llama, goat, rabbit, sheep, hamster, or guinea pig. In embodiments, the subject is a non-human primate such as, for example, monkey, chimpanzee, gorilla, orangutan, or a gibbon.

Depending on context, the terms “cell culture supplement” and “cell culture supplement composition” are used interchangeably.

Depending on context, the terms “cell culture medium” and “cell culture medium composition” are used interchangeably.

The term “oxidative phosphorylation” (OXPHOS) refers to the metabolic pathway in which cells use enzymes to oxidize nutrients, releasing energy used to reform ATP. This takes place inside mitochondria. Almost all aerobic organisms carry out oxidative phosphorylation. This pathway is a highly efficient way of releasing energy, compared to alternative fermentation processes such as anaerobic glycolysis. During oxidative phosphorylation, electrons are transferred from electron donors to electron acceptors such as oxygen, in redox reactions. These redox reactions release energy, which is used to form ATP. These redox reactions are carried out by a series of protein complexes within the inner membrane of the cell's mitochondria. These linked sets of proteins are called electron transport chains. The energy released by electrons flowing through this electron transport chain is used to transport protons across the inner mitochondrial membrane, in a process called electron transport.

The term “activation,” as used herein, refers to the state of a cell following sufficient cell surface moiety ligation to induce a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and up- or down-regulation of expression of cell surface molecules such as receptors or adhesion molecules, or up- or down-regulation of secretion of certain molecules, and performance of regulatory or cytolytic effector functions. Within the context of other cells, this term infers either up- or down-regulation of a particular physico-chemical process.

In embodiments, stimulation comprises a primary response induced by ligation of a cell surface moiety. For example, in the context of receptors, such stimulation may entail the ligation of a receptor and a subsequent signal transduction event. In embodiments, culturing T cells comprises stimulating the T cells. With respect to stimulation of a T cell, such stimulation may refer to the ligation of a T cell surface moiety that in embodiments subsequently induces a signal transduction event, such as binding the TCR/CD3 complex. In embodiments, the stimulation event may activate a cell and up- or down-regulate expression of cell surface molecules such as receptors or adhesion molecules, or up- or down-regulate secretion of a molecule, such as down regulation of Tumor Growth Factor beta (TGF-β) or up-regulation of IL-2, IFN-γ etc. In embodiments, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “ligand” or “stimulatory agent”, as used herein, refers to a molecule that binds to one or more defined population of cells (e.g., members of T cell subpopulations) and induces a cellular response. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. In embodiments, in the context of T cell stimulation, antibodies are used as a prototypical example of such an agent.

Antibodies for use in methods of the present invention may be of any species, class or subtype providing that such antibodies can react with the target of interest, e.g., CD3, the TCR, or CD28 as appropriate.

Thus “antibodies” for use in the present invention include:
(a) any of the various classes or sub-classes of immunoglobulin (e.g., IgG, IgA, IgM, IgD or IgE derived from any animal, e.g., any of the animals conventionally used, e.g., sheep, rabbits, goats, mice, camelids, or egg yolk),
(b) monoclonal or polyclonal antibodies,
(c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, VHH, or other single domain antibodies), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains;
(d) antibodies produced or modified by recombinant DNA or other synthetic techniques, including monoclonal antibodies, fragments of antibodies, “humanized antibodies”, chimeric antibodies, or synthetically made or altered antibody-like structures.

Also included are functional derivatives or “equivalents” of antibodies e.g., single chain antibodies, CDR-grafted antibodies etc. A single chain antibody (SCA) may be defined as a genetically engineered molecule containing the variable region of the light chain, the variable region of the heavy chain, linked by a suitable polypeptide linker as a fused single chain molecule.

Methods of preparation of antibody fragments and synthetic and derivatized antibodies are well known in the art and widely described in the literature and are not be described herein.

The term “differentiation”, as used herein, refers to a stage in development of the life cycle of a cell. T cells originate from hematopoietic stem cells in the bone marrow and generate a large population of immature thymocytes. The thymocytes (or T cells) progress from double negative cells to become double-positive thymocytes (CD4+ CD8+), and finally mature to single-positive (CD4+ CD8− or CD4−CD8+). During T cell differentiation, the naïve T cell becomes a blast cell that proliferates by clonal expansion and differentiates into memory and effector T cells. Many subsets of helper T cells (Th cells) are created during T cell differentiation and perform different functions for the immune system. In some embodiments, the differentiation stage of a T cell may be assessed through the presence or absence of markers including, but not limited to, CD3, CD4, CD5, CD8, CD11c, CD14, CD19, CD20, CD25, CD27, CD33, CD34, CD45, CD45RA, CD45RB, CD56, CD62L, CD123, CD127, CD278, CD335, CD11a, CD45RO, CD57, CD58, CD69, CD95, CD103, CD161, CCR7, as well as the transcription factors FOXP3, RORγ, T-bet, c-Rel, GATA3, etc.

The term “fluorescence-activated cell sorting (FACS)” as used herein, refers to a specialized type of flow cytometry. FACS provides a method for sorting a heterogeneous mixture of biological cells into two or more containers, one cell at a time, based upon the specific light scattering and fluorescent characteristics of each cell. FACS provides fast, objective and quantitative recording of fluorescent signals from individual cells as well as physical separation of cells of particular interest.

The term “proliferation” as used herein, means to grow or multiply by producing new cells.

The term “serum free media” as used herein, refers to cell culture media that does contain serum. “Low serum media” refers to cell culture media with a low percentage of serum supplementation (0.5-2% serum).

A “co-stimulatory signal,” as used herein, refers to a signal, which in combination with a primary signal, such as TCR/CD3 ligation, leads to T cell proliferation and/or activation and/or polarization.

“Separation,” as used herein, includes any means of substantially purifying one component from another (e.g., by filtration, affinity, buoyant density, or magnetic attraction).

As used, herein, the term “CD8+ T cell” refers to a T cell that presents the co-receptor CD8 on its surface. CD8 is a transmembrane glycoprotein that serves as a co-receptor for T cell receptor (TCR), which can recognize a specific antigen. Like the TCR, CD8 binds to a major histocompatibility complex I (MHC I) molecule. In embodiments, CD8+ T cells are cytotoxic CD8+ T cells (also known as cytotoxic T lymphocytes, T-killer cells, cytolytic T cells, or killer T cells). In embodiments, CD8+ T cells are regulatory CD8+ T cells, also referred to as CD8+ T cell suppressors.

As used, herein, the term “CD4+ T cell” refers to a T cell that presents the co-receptor CD4 on its surface. CD4 is a transmembrane glycoprotein that serves as a co-receptor for T cell receptor (TCR), which can recognize a specific antigen. In embodiments, CD4+ T cells are T helper cells. T helper cells (TH cells) assist other white blood cells in immunologic processes, including maturation of B cells into plasma cells and memory B cells, and activation of cytotoxic T cells and macrophages. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate different types of immune responses. Signaling from the APC directs T cells into particular subtypes. In embodiments, CD4+ T cells are regulatory T cells.

Included herein are methods for efficiently generating regulatory T cells (or “T regulatory cell” or “Treg”) and the use of these methods in the generation of T cell populations which have applications in, for example, immunotherapy. Treg cells can be characterized by markers, such as CD4+, CD25+, FOXP3+, CD127neg/low. In embodiments, Treg cells expanded using compositions and methods provided herein are CD4+, CD25+, FOXP3−. Non-limiting examples of compositions and methods for generating FOXP3− regulatory T cells are set out in Aarvak et al., U.S. Pat. No. 9,119,807.

Without being bound by any scientific theory, naturally occurring regulatory T (Treg) cells negatively regulate the activation of other T cells, including effector T cells, as well as innate immune system cells and can be utilized in immunotherapy against autoimmune diseases and provide transplantation tolerance. Various populations of Treg cells have been described and include naturally occurring CD4+ CD25+FOXP3+ cells and induced Tr1 and Th3 cells that secrete IL-10 and TGF-β respectively.

Treg cells are characterized by sustained suppression of effector T cell responses. Traditional or conventional Treg cells can be found, e.g., in the spleen or the lymph node or in the circulation. Tregs are proven highly effective in preventing GVHD and autoimmunity in murine models. Clinical trials with adoptive transfer of Tregs in transplantation, treatment of diabetes and other indications are underway. The relative frequency of Tregs in peripheral blood is approximately 1-2% of total lymphocytes implicating the necessity of ex vivo expansion of Tregs prior to adoptive transfer for most clinical applications. Producing sufficient Tregs during the ex vivo expansion has been a major challenge in applying Treg therapy to humans.

T helper 17 cells (or “Th17 cells” or “Th17 helper cells”) are an inflammatory subset of CD4+ T helper cells that regulate host defense, and are involved in tissue inflammation and various autoimmune diseases. Th17 cells have been found in various human tumors however their function in cancer immunity is unclear. When adoptively transferred into tumor-bearing mice, Th17 cells have been found to be more potent at eradicating melanoma than Th1 or non-polarized (Th0) T cells (Muranski et al. Blood. 2008). Th17 cells are developmentally distinct from Th1 and Th2 lineages. Th17 cells are CD4+ cells that are responsive to IL-1R1 and IL-23R signaling and produce the cytokines IL-17A, IL-17F, IL-17AF, IL-21, IL-22, IL-26 (human), GM-CSF, MIP-3a, and TNFα. The phenotype of Th17 cells is controversial but currently defined as CD3+, CD4+, CCR4+, CCR6+ or CD3+, CD4+, CCR6+, CXCR3+. One obstacle to the use of Th17 cells for adoptive cell transfer has been the identification of robust culture conditions that can expand the Th17 cell subset.

Included herein are compositions and methods for the generation of T cell subtypes. One T cell subtype that may be produced using compositions and methods of the invention are Th17 cells.

T helper 9 cells (or “Th9 cells” or “Th9 helper cells”) are an inflammatory subset of CD4+ T helper cells that regulate host defense and are involved in allergy, inflammation and various autoimmune diseases. Th9 cells are identified by secretion of the signature cytokine IL-9. Although Th9 cells share some functional roles with Th2 cells, including promoting allergic inflammation and helminthic parasite immunity, Th9 cells can also promote autoimmunity in responses that are generally characterized as dependent on Th1 or Th17 cells. Th9 cells are differentiated under a cytokine environment containing both IL-4 and transforming growth factor β (TGFβ), which induce the transcriptional network required for the expression of IL-9. The Th9 subset is defined by its ability to produce large amounts of the signature cytokine IL-9. Transcription factors required for the development of Th9 cells include signal transducer and activator of transcription-6 (STAT6), interferon regulatory factor 4 (IRF4), B-cell activating transcription factor-like (BATF), GATA3, PU.1 and Smads. Th9 cells express high levels of IL-25 receptor (IL17RB), which is a potential surface maker to distinguish Th9 cells from other T helper subsets. Immune responses mediated by Th9 cells contribute to the protective immunity against intestinal parasite infection and to anti-tumor immunity.

Provided herein are compositions and methods for the generation of T cell subtypes. A non-limiting example of a T cell subtype that may be produced using compositions and methods of the invention are Th9 cells.

Memory T cells, or antigen-experienced cells, are experienced in a prior encounter with an antigen. These T cells are long-lived and can recognize antigens and quickly and strongly affect an immune response to an antigen to which they have been previously exposed. Memory T cells can encompass: stem memory cells (TSCM), central memory cells (TCM), effector memory cells (TEM,). TSCM cells have the phenotype CD45RO−, CCR7+, CD45RA+, CD62L+(L-selectin), CD27+, CD28+ and IL-7Ra+, but they also express large amounts of IL-2Rβ, CXCR3, and LFA-1. TCM cells express L-selectin and CCR7, and they secrete IL-2. TEM cells do not express L-selectin or CCR7 but produce effector cytokines like IFN-γ and IL-4.

Included herein are methods and compositions for the expansion of T cell populations.

“Chimeric antigen receptor” or “CAR” or “CARs” as used herein refers to engineered receptors, which graft an antigen specificity onto cells (for example T cells such as naïve T cells, central memory T cells, effector memory T cells or any combination thereof). CARs are also known as artificial T cell receptors, chimeric T cell receptors or chimeric immunoreceptors. In embodiments, a CAR comprises one or more antigen-specific targeting domains, an extracellular domain, a transmembrane domain, one or more co-stimulatory domains, and an intracellular signaling domain. In embodiments, if the CAR targets two different antigens, the antigen-specific targeting domains may be arranged in tandem. In embodiments, if the CAR targets two different antigens, the antigen-specific targeting domains may be arranged in tandem and separated by linker sequences.

CARs are engineered receptors, which graft an arbitrary specificity onto an immune effector cell (T cell). These receptors are used to graft the specificity of a monoclonal antibody onto a T cell; with transfer of their coding sequence facilitated by retroviral vectors. The receptors are called chimeric because they are composed of parts from different sources. CARs may be used as a therapy for cancer through adoptive cell transfer. T cells are removed from a patient and modified so they express receptors specific to the patient's particular cancer. The T cells, which recognize and kill the cancer cells, are reintroduced into the patient. In embodiments, modification of T cells sourced from donors other than the patient may be used to treat the patient.

Using adoptive transfer of T cells expressing chimeric antigen receptors, CAR-modified T cells can be engineered to target any tumor-associated antigen. Following the collection of a patient's T cells, the cells are genetically engineered to express CARs specifically directed towards antigens on the patient's tumor cells before being infused back into the patient.

A method for engineering CAR T cells for cancer immunotherapy is to use viral vectors such as retrovirus, lentivirus or transposon, which integrate the transgene into the host cell genome. Alternatively, non-integrating vectors such as plasmids or mRNA may be used but these types of episomal DNA/RNA may be lost after repeated cell division. Consequently, the engineered CAR T cells may eventually lose their CAR expression. In another approach, a vector is used that is stably maintained in the T cell, without being integrated in its genome. This strategy has been found to enable long-term transgene expression without the risk of insertional mutagenesis or genotoxicity.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Media and Media Supplements

In an aspect, provided herein is cell culture medium (e.g., for T cells) that includes a cyclodextrin and at least one lipid (e.g., about or at least about 1, 2, 3, 4, 5, 6, 7, 8, 9, 10, 15, or 20 lipids). In embodiments, the cell culture medium includes (1) a cyclodextrin and (2) at least 2 lipids, at least 3 lipids, at least 4 lipids, at least 5 lipids, at least 6 lipids, at least 7 lipids, at least 8 lipids, at least 9 lipids, at least 10 lipids, at least 15 lipids, or at least 20 lipids (e.g., from about 3 to about 20, from about 4 to about 20, from about 5 to about 20, from about 6 to about 20, from about 7 to about 20, from about 3 to about 15, from about 3 to about 12, from about 3 to about 10, from about 3 to about 8, from about 5 to about 20, from about 5 to about 15, from about 5 to about 12, from about 5 to about 9, etc. fatty acids).). In another aspect, the cell culture medium includes a cyclodextrin and at least one lipid (e.g., about or at least about 1, 2, 3, 4, 5, 6, 7, 8, 9, 10, 15, or 20 lipids), and/or 2-deoxy-D-glucose (2-DG).

In an aspect, provided herein is cell culture medium supplement for cells (e.g., for T cells) that includes a cyclodextrin, at least one lipid (e.g., about or at least about 1, 2, 3, 4, 5, 6, 7, 8, 9, 10, 15, or 20 lipids), and/or 2-deoxy-D-glucose (2-DG). In embodiments, the cell culture medium supplement includes (1) a cyclodextrin, (2) at least 2 lipids, at least 3 lipids, at least 4 lipids, at least 5 lipids, at least 6 lipids, at least 7 lipids, at least 8 lipids, at least 9 lipids, at least 10 lipids, at least 15 lipids, or at least 20 lipids (e.g., from about 3 to about 20, from about 4 to about 20, from about 5 to about 20, from about 6 to about 20, from about 7 to about 20, from about 3 to about 15, from about 3 to about 12, from about 3 to about 10, from about 3 to about 8, from about 5 to about 20, from about 5 to about 15, from about 5 to about 12, from about 5 to about 9, etc. fatty acids), and/or 2-deoxy-D-glucose (2-DG).

In embodiments, the at least one lipid (1) is or (2) is select from the group consisting: of cholesterol, a fatty acid, a fatty acid ester, a phospholipid, and/or a glycerolipid. In embodiments, the at least one lipid is cholesterol. In embodiments, the at least one lipid is a fatty acid. In embodiments, the at least one lipid is a fatty acid ester. In embodiments, the at least one lipid is a phospholipid. In embodiments, the at least one lipid is a glycerolipid. In some embodiments, the at least one lipid two or more lipids select from the group consisting: of cholesterol, a fatty acid, a fatty acid ester, a phospholipid, and/or a glycerolipid.

In embodiments, the fatty acid is a saturated fatty acid, a monounsaturated fatty acid, or a polyunsaturated fatty acid. In embodiments, the fatty acid is a saturated fatty acid. In embodiments, the fatty acid is a monounsaturated fatty acid. In embodiments, the fatty acid is a polyunsaturated fatty acid.

In embodiments, the fatty acid is a monounsaturated fatty acid. In embodiments, the fatty acid is a polyunsaturated fatty acid. In some embodiments, the fatty acid is one or more fatty acid type selected from the group consisting of: (1) a saturated fatty acid, (2) a monounsaturated fatty acid, and (1) a polyunsaturated fatty acid, as well as combinations of such fatty acids (e.g., two polyunsaturated fatty acids, three monounsaturated fatty acid, and one saturated fatty acid).

In embodiments, the fatty acid is an omega-3 fatty acid, an omega-6 fatty acid, or an omega-9 fatty acid, or a combination of one or more (e.g., two or more or three) of an omega-3 fatty acid, an omega-6 fatty acid, or an omega-9 fatty acid. In embodiments, the fatty acid is a long chain polyunsaturated fatty acids (LC-PUFA). In embodiments, the fatty acid is a saturated fatty acid. In embodiments, the fatty acid is a monounsaturated fatty acid. In embodiments, the fatty acid is a polyunsaturated fatty acid.

In some embodiments, the medium or supplement comprises linoleic acid, at least one other omega-6 fatty acid, cholesterol, a methylated cyclodextrin. In other embodiments, the medium or supplement comprises: (i) linoleic acid, at least one other omega-6 fatty acid, cholesterol, a methylated cyclodextrin, and/or (ii) 2-deoxy-D-glucose (2-DG).

In some embodiments, the cell culture medium further includes one or more fatty acids selected from the group consisting of: (1) linoleic acid (2) linolenic acid, (3) arachidonic acid, (4) myristic acid, (5) oleic acid, (6) palmitic acid, (7) palmitoleic acid, (8) stearic acid, (9) oleic acid, and (10) palmitic acid. In specific embodiments, the cell culture medium further includes one or more fatty acids selected from the group consisting of: (1) linoleic acid and/or (2) linolenic acid. In some embodiments, the linolenic acid is alpha-linolenic acid, gamma-linolenic acid, and/or alpha-linolenic acid and gamma-linolenic acid. In some embodiments, the linolenic acid is alpha-linolenic acid. In embodiments, the linolenic acid is gamma-linolenic acid. In embodiments, the linolenic acid is alpha-linolenic acid and gamma-linolenic acid.

In embodiments, the cell culture medium further includes arachidonic acid. In embodiments, the cell culture medium supplement further includes arachidonic acid.

In embodiments, the cell culture medium further includes myristic acid, oleic acid, palmitic acid, palmitoleic acid, and/or stearic acid. In embodiments, the cell culture medium further includes myristic acid. In embodiments, the cell culture medium further includes oleic acid. In embodiments, the cell culture medium further includes palmitic acid. In embodiments, the cell culture medium further includes palmitoleic acid. In embodiments, the cell culture medium further includes stearic acid.

In embodiments, the cell culture medium supplement further includes myristic acid, oleic acid, palmitic acid, palmitoleic acid, and/or stearic acid. In embodiments, the cell culture medium supplement further includes myristic acid. In embodiments, the cell culture medium supplement further includes oleic acid. In embodiments, the cell culture medium supplement further includes palmitic acid. In embodiments, the cell culture medium supplement further includes palmitoleic acid. In embodiments, the cell culture medium supplement further includes stearic acid.

In embodiments, the at least one lipid is: (a) any one of linoleic acid, linolenic acid, arachidonic acid, myristic acid, oleic acid, palmitic acid, palmitoleic acid, or stearic acid; (b) 2, 3, 4, 5, 6, or 7 of any of linoleic acid, linolenic acid, arachidonic acid, myristic acid, oleic acid, palmitic acid, palmitoleic acid, and stearic acid; (c) a mixture of linoleic acid, linolenic acid, arachidonic acid, myristic acid, oleic acid, palmitic acid, palmitoleic acid, and stearic acid; (d) a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid; (e) monounsaturated fatty acid, where the monounsaturated fatty acid is palmitoleic acid, oleic acid, eicosenoic acid, erucic acid, or nervonic acid; (f) polyunsaturated fatty acid, and the polyunsaturated fatty acid is hexadecatrienoic acid, linoleic acid, alpha-linolenic acid, gamma-linolenic acid, stearidonic acid, eicosadienoic acid, eicosatrienoic acid, dihomo-gamma-linolenic acid, mead acid, arachidonic acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid; (g) omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid; (h) omega-6 fatty acid, and the omega-6 fatty acid is linoleic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid; or (i) omega-9 fatty acid, and the omega-9 fatty acid is palmitoleic acid, oleic acid, eicosenoic acid, erucic acid, nervonic acid, or mead acid. In embodiments, the at least one lipid is any one of linoleic acid, linolenic acid, arachidonic acid, myristic acid, oleic acid, palmitic acid, palmitoleic acid, or stearic acid. In embodiments, the at least one lipid is 2, 3, 4, 5, 6, or 7 of linoleic acid, linolenic acid, arachidonic acid, myristic acid, oleic acid, palmitic acid, palmitoleic acid, and stearic acid. In embodiments, the at least one lipid is linoleic acid, linolenic acid, arachidonic acid, myristic acid, oleic acid, palmitic acid, palmitoleic acid, and stearic acid. In embodiments, the at least one lipid is a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid. In embodiments, the at least one lipid is monounsaturated fatty acid, and the monounsaturated fatty acid is palmitoleic acid, oleic acid, eicosenoic acid, erucic acid, or nervonic acid. In embodiments, the at least one lipid is polyunsaturated fatty acid, and the polyunsaturated fatty acid is hexadecatrienoic acid, linoleic acid, alpha-linolenic acid, gamma-linolenic acid, stearidonic acid, eicosadienoic acid, eicosatrienoic acid, dihomo-gamma-linolenic acid, mead acid, arachidonic acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid. In embodiments, the at least one lipid is omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid. In embodiments, the at least one lipid is omega-6 fatty acid, and the omega-6 fatty acid is linoleic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid. In embodiments, the at least one lipid is omega-9 fatty acid, and the omega-9 fatty acid is palmitoleic acid, oleic acid, eicosenoic acid, erucic acid, nervonic acid, or mead acid.

In embodiments, the at least one lipid is cholesterol. In embodiments, the cholesterol is a synthetic cholesterol. In embodiments, the cholesterol is free cholesterol.

In some embodiments, the cyclodextrin is an α-cyclodextrin, a β-cyclodextrin, and/or a γ-cyclodextrin. In embodiments, the cyclodextrin is an α-cyclodextrin. In embodiments, the cyclodextrin is a β-cyclodextrin. In embodiments, the cyclodextrin is a γ-cyclodextrin. In embodiments, the cyclodextrin is methylated. In embodiments, the cyclodextrin is methyl-β-cyclodextrin.

In embodiments, the cyclodextrin is one or more of the following: 2-hydroxypropyl-β-cyclodextrin, sulfobutylether-β-cyclodextrin, 2-hydroxypropyl-γ-cyclodextrin, 2,6-dimethyl-α-cyclodextrin, hydroxypropyl-γ-cyclodextrin, hydroxyethyl-β-cyclodextrin, β-cyclodextrin polysulfate, trimethyl β-cyclodextrin, and/or γ-cyclodextrin polysulfate. In embodiments, the cyclodextrin is 2-hydroxypropyl-β-cyclodextrin, sulfobutylether-β-cyclodextrin, 2-hydroxypropyl-γ-cyclodextrin, 2,6-dimethyl-α-cyclodextrin, hydroxypropyl-γ-cyclodextrin, hydroxyethyl-β-cyclodextrin, β-cyclodextrin polysulfate, trimethyl β-cyclodextrin, and/or γ-cyclodextrin polysulfate.

In embodiments, the composition includes a plurality of different cyclodextrins, wherein the plurality of cyclodextrins includes at least two cyclodextrins (e.g., about or at least about 2, 3, 4, 5, 6, 7, 8, 9, or 10 cyclodextrins). In embodiments, the composition includes different cyclodextrins ranging from about two to about ten (e.g., from about two to about ten, from about three to about ten, from about four to about ten, from about five to about ten, from about two to about eight, from about three to about eight, from about four to about eight from about two to about ten, etc.).

In embodiments, the plurality of cyclodextrins includes at least one (e.g., about or at least about 1, 2, 3, 4, 5, 6, 7, 8, 9, or 10) α-cyclodextrin, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 2 α-cyclodextrins, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 3 α-cyclodextrins, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 4 α-cyclodextrins, at least one (e.g., about or at least about 1, 2, 3, 4, 5, 6, 7, 8, 9, or 10) β3-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 5 α-cyclodextrins, at least one (e.g., about or at least about 1, 2, 3, 4, 5, 6, 7, 8, 9, or 10) β3-cyclodextrin, and/or at least one (e.g., about or at least about 1, 2, 3, 4, 5, 6, 7, 8, 9, or 10) γ-cyclodextrin.

In embodiments, the cell culture medium includes linoleic acid, cholesterol, and the cyclodextrin.

In embodiments, the cell culture medium supplement includes linoleic acid, cholesterol, and the cyclodextrin.

In embodiments, at least about 5%, 10%, 15%, 20%, 25%, 30%, 35%, 40%, 45%, 50%, 55%, 60%, 65%, 70%, 75%, 80%, 85%, 90%, 98% (e.g., from about 5% to about 50%, from about 5% to about 50%, from about 5% to about 50%, from about 5% to about 98%, from about 5% to about 85%, from about 5% to about 75%, from about 5% to about 60%, from about 10% to about 98%, from about 15% to about 95%, from about 20% to about 95%, from about 40% to about 80%, from about 65% to about 99%, etc.) of the cholesterol molecules in a composition or combination are within (e.g., at least a portion thereof is inside of) the ring of a cyclodextrin molecule.

In embodiments, at least about 5%, 10%, 15%, 20%, 25%, 30%, 35%, 40%, 45%, 50%, 55%, 60%, 65%, 70%, 75%, 80%, 85%, 90%, 98% (e.g., from about 5% to about 50%, from about 5% to about 50%, from about 5% to about 50%, from about 5% to about 98%, from about 5% to about 85%, from about 5% to about 75%, from about 5% to about 60%, from about 10% to about 98%, from about 15% to about 95%, from about 20% to about 95%, from about 40% to about 80%, from about 65% to about 99%, etc.) of the fatty acid molecules in a composition or combination are within (e.g., at least a portion thereof is inside of) the ring of a cyclodextrin molecule.

In an aspect, subject matter provided herein relates to formulations with defined ratios, such as cyclodextrin/fatty acid ratios. A number of factors will determine ratios of components of such formulations, such as cyclodextrin toxicity, the fatty acid “carrying capacity” of the particular cyclodextrin or cyclodextrins used, and the desired effect on the particular cell population that cyclodextrin/fatty acid formulation is used in conjunction with. In another aspect, subject matter provided herein relates to formulations with defined ratios, such as cyclodextrin/fatty acid/2-deoxy-D-glucose (2-DG) ratios or cyclodextrin/lipid/2-deoxy-D-glucose (2-DG) ratios.

A formula that may be used to describe the relative amount (i.e., ratio) of one compound (or group of compounds) to another within a formulation is X:X, wherein each X is individually a specifically named compound or class of compounds. For example, each X may be, individually, (1) the total amount of cyclodextrin(s) present in a formulation (TC), a specifically named cyclodextrin present in a formulation, or a class of cyclodextrins present in a formulation; (2) the total amount of fatty acid(s) present in a formulation (TFA), a specifically named fatty acid present in a formulation, or a class of fatty acids present in a formulation; or (3) the total amount of cholesterol present in a formulation (TCOL), a specifically named cholesterol present in a formulation, or a class of cholesterols present in a formulation.

Optionally, a formula that may be used to describe the relative amounts of cyclodextrin(s) and fatty acid(s) in a formulation (such as a cell culture medium or a cell culture supplement) is TC:TFA, where TC represents the total amount of cyclodextrin(s) present and TFA represents the total amount of fatty acid(s) present. Alternatively, the formula TFA:TC may be used. One convenient way of setting out values for such formulas is through the use of molar amounts and, in particular, molar ratios. In embodiments, suitable molar ratios of TC:TFA range from 1:0.001 to 1:0.5 (e.g., from about 1:0.01 to about 1:0.4, from about 1:0.01 to about 1:0.3, from about 1:0.01 to about 1:0.2, from about 1:0.01 to about 1:0.15, from about 1:0.01 to about 1:0.1, from about 1:0.05 to about 1:0.1, from about 1:0.05 to about 1:0.08, from about 1:0.02 to about 1:0.2, from about 1:0.02 to about 1:0.1, from about 1:0.02 to about 1:0.08, from about 1:0.04 to about 1:0.2, etc.). Additional non-limiting examples of ratios are disclosed herein.

The amount of total cyclodextrin (TC) may be represented by a single cyclodextrin or two or more cylodextrins. Further, when more than one cyclodextrin is present, the amount of these cyclodextrins may be the same or different. In embodiments, the molar ratio of 1 cyclodextrin to 1 or more other cyclodextrins (e.g., all other cyclodextrins present in a formulation) may be, e.g., from 1:0.1 to 1:10 (e.g., from about 1:0.1 to about 1:5, from about 1:0.2 to about 1:5, from about 1:0.3 to about 1:5, from about 1:0.4 to about 1:5, from about 1:0.5 to about 1:5, from about 1:1 to about 1:10, from about 1:2 to about 1:10, from about 1:3 to about 1:10, from about 1:4 to about 1:10, from about 1:5 to about 1:10, etc.).

In many instances, more than one fatty acid will be present in a formulation (such as a cell culture medium or a cell culture supplement). Further, when more than one fatty acid is present in a formulation provided herein, these fatty acids may be present in the same or different amounts. Tables 1-3 set out exemplary formulations of provided herein and exemplary molar ratios of components.

In embodiments, at least some fatty acids present in formulations provided herein will be present in differing amounts.

In embodiments, a cell culture medium and/or cell culture medium supplement includes cyclodextrin and cholesterol, wherein the molar ratio of TC to TCOL is less than 10.5:1, less than 10:1, less than 9.5:1, less than 9:1, less than 8.5:1, less than 8:1, less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1. The molar ratio of TC to the TCOL may thus be from about 10.5:1 to about 0.1:1, from about 8:1 to about 0.1:1, from about 7:1 to about 0.1:1, from about 6:1 to about 0.1:1, from about 5:1 to about 0.1:1, from about 3:1 to about 0.1:1, from about 2:1 to about 0.1:1, etc.

In embodiments, a cell culture medium and/or culture medium supplement includes a cyclodextrin and at least one fatty acid, wherein the molar ratio of TC to TFA is less than 11.5:1, less than 11:1, less than 10.5:1, less than 10:1, less than 9.5:1, less than 9:1, less than 8.5:1, less than 8:1, less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1. The molar ratio of the cyclodextrin to the at least one fatty acid may thus be from about 11.5:1 to about 0.1:1, from about 10.5:1 to about 0.1:1, from about 8:1 to about 0.1:1, from about 7:1 to about 0.1:1, from about 6:1 to about 0.1:1, from about 5:1 to about 0.1:1, from about 3:1 to about 0.1:1, from about 2:1 to about 0.1:1, etc.

In embodiments, the cell culture medium and/or culture medium supplement includes a cyclodextrin, cholesterol, and at least one fatty acid, wherein the molar ratio of (1) TC to (2) TCOL and TFA (e.g., the sum of the molar values of TCOL and TFA) is less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1. The molar ratio of (1) TC to (2) the TCOL and TFA may thus be from about 7:1 to about 0.1:1, from about 6:1 to about 0.1:1, from about 5:1 to about 0.1:1, from about 3:1 to about 0.1:1, from about 2:1 to about 0.1:1, etc.

In embodiments, the ratio of TC to TCOL on a molar basis is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of TFA to TC on a molar basis is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of TFA on a molar basis to TCOL is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of total polyunsaturated fatty acid molecules on a molar basis to other fatty acid molecules is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of total omega-3, omega-6, and/or omega-9 polyunsaturated fatty acid molecules on a molar basis to other fatty acid molecules is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In some embodiments, multiple hydrophobic compounds with immune activity are incorporated in a lipid supplement, including prostaglandins, corticosteroids, leukotrienes, lipoxins, protectins and resolvins. Also, lipid drugs such as Etomoxir and statins. Etomoxir is a chemical entity shown to have activities associated with irreversible O-carnitine palmitoyltransferase-1 (CPT-1) inhibition and PPARα activation. Additional compounds that may be present in and used in methods of the invention are chemical entities having CPT-1 inhibition and/or PPARα activation activity.

In some embodiments, oligonucleotides are loaded in cyclodextrins. For a non-limiting review of cyclodextrin use in drug delivery, see Chordiya and Senthilkumaran (2012) Research and Reviews: Journal of Pharmacy and Pharmaceutical Sciences 1(1):19-29 (especially Table 2 thereof as it describes different cycodextrins in use as carriers), the entire content of which is incorporated herein by reference.

In embodiments, the cell culture medium further includes a prostaglandin, a corticosteroid, a leukotriene, a lipoxin, a protectin, a resolvin, an oligonucleotide, or hydrophobic drug compound. In embodiments, the cell culture medium further includes a prostaglandin. In embodiments, the cell culture medium further includes a corticosteroid. In embodiments, the cell culture medium further includes a leukotriene. In embodiments, the cell culture medium further includes a lipoxin. In embodiments, the cell culture medium further includes a protectin. In embodiments, the cell culture medium further includes a resolvin. In embodiments, the cell culture medium further includes an oligonucleotide. In embodiments, the cell culture medium further includes a hydrophobic drug compound.

In embodiments, the cell culture medium supplement further includes a prostaglandin, a corticosteroid, a leukotriene, a lipoxin, a protectin, a resolvin, an oligonucleotide, or hydrophobic drug compound. In embodiments, the cell culture medium supplement further includes a prostaglandin. In embodiments, the cell culture medium supplement further includes a corticosteroid. In embodiments, the cell culture medium supplement further includes a leukotriene. In embodiments, the cell culture medium supplement further includes a lipoxin. In embodiments, the cell culture medium supplement further includes a protectin. In embodiments, the cell culture medium supplement further includes a resolvin. In embodiments, the cell culture medium supplement further includes an oligonucleotide. In embodiments, the cell culture medium supplement further includes a hydrophobic drug compound.

In embodiments, the hydrophobic drug compound is etomoxir or a statin. In embodiments, the hydrophobic drug compound is etomoxir. In embodiments, the hydrophobic drug compound is a statin.

In embodiments, the cell culture medium comprises 2-DG. In embodiments, the cell culture medium supplement comprises 2-DG.

In embodiments, the cell culture medium includes a level of 2-DG that is about 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 3 mM, 4 mM, 5 mM, 6 mM, 7 mM, 8 mM, 9 mM, or 10 mM. In embodiments, the cell culture medium includes a level of 2-DG from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.25 mM to about 5 mM, from about 0.25 mM to about 5 mM, from about 0.25 mM to about 4 mM, from about 0.25 mM to about 3 mM, from about 0.25 mM to about 2 mM, or from about 0.25 mM to about 1 mM. In embodiments, the level of 2-DG is less than 5 mM, 4 mM, 3 mM, 2 mM, 1 mM, 0.5 mM, or 0.25 mM. In embodiments, the level of 2-DG is about 5 mM, 4 mM, 3 mM, 2 mM, 1 mM, 0.5 mM, or 0.25 mM.

In embodiments, the cell culture medium includes a level of cyclodextrin that is about 200 μM, 150 μM, 140 μM, 130 μM, 120 μM, 110 μM, 100 μM, 90 μM, 80 μM, 70 μM, 60 μM, 50 μM, 40 μM, 30 μM, 20 μM, 10 μM, 5 μM, 1 μM, 0.5 μM, 0.25 μM, 0.1 μM, 0.05 μM, 0.025 μM, 0.001 μM, less than 200 μM, less than 150 μM, less than 140 μM, less than 130 μM, less than 120 μM, less than 110 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM, less than 1 μM, less than 0.5 μM, less than 0.25 μM, less than 0.1 μM, less than 0.05 μM, less than 0.025 μM, or less than 0.01 μM.

In embodiments, the cell culture medium includes a level of cyclodextrin that is from about 50 μM to about 200 μM, from about 55 μM to about 195 μM, from about 50 μM to about 190 μM, from about 65 μM to about 185 μM, from about 70 μM to about 180 μM, from about 75 μM to about 175 μM, from about 80 μM to about 170 μM, from about 85 μM to about 165 μM, from about 90 μM to about 160 μM, from about 90 μM to about 155 μM, from about 95 μM to about 150 μM, from about 100 μM to about 145 μM, from about 105 μM to about 140 μM, from about 110 μM to about 135 μM, from about 115 μM to about 130 μM, or from about 120 μM to about 125 μM.

In embodiments, the cell culture medium includes a level of at least one lipid that is at least about 5 μM, 10 μM, 15 μM, 20 μM, 25 μM, 30 μM, 35 μM, 40 μM, 45 μM, or 50 μM. In embodiments, the cell culture medium includes a level of at least one lipid that is from about 5 μM, 10 μM, or 15 μM to about 20 μM, 25 μM, 30 μM, 35 μM, 40 μM, 45 μM, or 50 μM. In embodiments, the cell culture medium includes a level of at least one lipid that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 19 μM to about 21 μM. In embodiments, the level of the at least one lipid in the cell culture medium is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium includes a level of at least one fatty acid that is at least about 5 μM, 10 μM, 15 μM, 20 μM, 25 μM, 30 μM, 35 μM, 40 μM, 45 μM, or 50 μM. In embodiments, the cell culture medium includes a level of at least one fatty acid that is from about 5 μM, 10 μM, or 15 μM to about 20 μM, 25 μM, 30 μM, 35 μM, 40 μM, 45 μM, or 50 μM. In embodiments, the cell culture medium includes a level of at least one fatty acid that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 19 μM to about 21 μM. In some embodiments, the level of the at least one fatty acid in the cell culture medium is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium includes a level of at least one polyunsaturated fatty acid (e.g., an omega-6 fatty acid such as arachidonic acid, linoleic acid, and/or gamma-linolenic acid) that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of at least one polyunsaturated fatty acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of at least one polyunsaturated fatty acid that is from about 0.05 μM to about 50 μM, from about 0.5 μM to about 10 μM, from about 5 μM to about 10 μM, or from about 1 μM to about 5 μM.

In embodiments, the cell culture medium includes a level of arachidonic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of arachidonic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of arachidonic acid that is from about 0.05 μM to about 50 μM, from about 5 μM to about 10 μM, from about 0.5 μM to about 10 μM, or from about 1 μM to about 5 μM.

In embodiments, the cell culture medium includes a level of linoleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linoleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linoleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of linolenic that (e.g., alpha-linolenic acid, gamma-linolenic acid, or a combination thereof) is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linolenic that that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linolenic that that is from about 0.05 μM to about 50 μM, from about 0.5 μM to about 10 μM, from about 5 μM to about 10 μM, or from about 1 μM to about 5 μM.

In embodiments, the cell culture medium includes a level of at least one fatty acid other than a polyunsaturated fatty acid (e.g., a saturated fatty acid such as myristic acid, palmitic acid or stearic acid, and/or a monounsaturated fatty acid such as palmitoleic acid or oleic acid) that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of the fatty acid(s) that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of the fatty acid(s) that is from about 0.05 μM to about 50 μM, from about 0.5 μM to about 10 μM, from about 5 μM to about 10 μM, or from about 1 μM to about 5 μM.

In embodiments, the cell culture medium includes a level of myristic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of myristic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of myristic acid that is from about 0.05 μM to about 50 μM, from about 0.5 μM to about 10 μM, from about 5 μM to about 10 μM, from about 1 μM to about 5 μM.

In embodiments, the cell culture medium includes a level of palmitic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitic acid that is from about 0.05 μM to about 50 μM, from about 0.5 μM to about 10 μM, from about 5 μM to about 10 μM, or from about 1 μM to about 5 μM.

In embodiments, the cell culture medium includes a level of stearic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of stearic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of stearic acid that is from about 0.05 μM to about 50 μM, from about 0.5 μM to about 10 μM, from about 5 μM to about 10 μM, or from about 1 μM to about 5 μM.

In embodiments, the cell culture medium includes a level of palmitoleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitoleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitoleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of oleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of oleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of oleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of cholesterol that is at least about 5 μM, 10 μM, 15 μM, 20 μM, 25 μM, 30 μM, 35 μM, 40 μM, 45 μM, or 50 μM. In embodiments, the cell culture medium includes a level of cholesterol that is from about 5 μM, 10 μM, or 15 μM to about 20 μM, 25 μM, 30 μM, 35 μM, 40 μM, 45 μM, or 50 μM. In embodiments, the cell culture medium includes a level of cholesterol that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 18 μM to about 21 μM, from about 19 μM to about 20 μM. In embodiments, the cell culture medium includes a level of cholesterol that is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium does not include a drug compound. In embodiments, the cell culture medium supplement does not include a drug compound.

In embodiments, the cell culture medium does not include alprostadil, cefotiam hexetil HCl, benexate HCl, dexamethasone, iodine, nicotine, nimesulide, nitroglycerin, omeprazol, PGE2, piroxicam, tiaprofenic acid, cisapride, hydrocortisone, ludomethacin, itraconazole, mitomycin, 17β-estradiol, chloramphenicol, voriconazole, ziprasidoue maleate, diclofenac sodium, etomoxir or a statin. In embodiments, the cell culture medium does not include alprostadil. In embodiments, the cell culture medium does not include cefotiam hexetil HCl. In embodiments, the cell culture medium does not include benexate HCl. In embodiments, the cell culture medium does not include dexamethasone. In embodiments, the cell culture medium does not include iodine. In embodiments, the cell culture medium does not include nicotine. In embodiments, the cell culture medium does not include nimesulide. In embodiments, the cell culture medium does not include nitroglycerin. In embodiments, the cell culture medium does not include nitroglycerin. In embodiments, the cell culture medium does not include omeprazol. In embodiments, the cell culture medium does not include PGE2. In embodiments, the cell culture medium does not include piroxicam. In embodiments, the cell culture medium does not include tiaprofenic acid. In embodiments, the cell culture medium does not include cisapride. In embodiments, the cell culture medium does not include hydrocortisone. In embodiments, the cell culture medium does not include ludomethacin. In embodiments, the cell culture medium does not include itraconazole. In embodiments, the cell culture medium does not include mitomycin. In embodiments, the cell culture medium does not include 17β-estradiol. In embodiments, the cell culture medium does not include chloramphenicol. In embodiments, the cell culture medium does not include voriconazole. In embodiments, the cell culture medium does not include ziprasidoue maleate. In embodiments, the cell culture medium does not include diclofenac sodium. In embodiments, the cell culture medium does not include etomoxir. In embodiments, the cell culture medium does not include a statin.

In embodiments, the cell culture medium supplement does not include alprostadil, cefotiam hexetil HCl, benexate HCl, dexamethasone, iodine, nicotine, nimesulide, nitroglycerin, omeprazol, PGE2, piroxicam, tiaprofenic acid, cisapride, hydrocortisone, ludomethacin, itraconazole, mitomycin, 17β-estradiol, chloramphenicol, voriconazole, ziprasidoue maleate, diclofenac sodium, etomoxir or a statin. In embodiments, the cell culture medium supplement does not include alprostadil. In embodiments, the cell culture medium supplement does not include cefotiam hexetil HCl. In embodiments, the cell culture medium supplement does not include benexate HCl. In embodiments, the cell culture medium supplement does not include dexamethasone. In embodiments, the cell culture medium supplement does not include iodine. In embodiments, the cell culture medium supplement does not include nicotine. In embodiments, the cell culture medium supplement does not include nimesulide. In embodiments, the cell culture medium supplement does not include nitroglycerin. In embodiments, the cell culture medium supplement does not include nitroglycerin. In embodiments, the cell culture medium supplement does not include omeprazol. In embodiments, the cell culture medium supplement does not include PGE2. In embodiments, the cell culture medium supplement does not include piroxicam. In embodiments, the cell culture medium supplement does not include tiaprofenic acid. In embodiments, the cell culture medium supplement does not include cisapride. In embodiments, the cell culture medium supplement does not include hydrocortisone. In embodiments, the cell culture medium supplement does not include ludomethacin. In embodiments, the cell culture medium supplement does not include itraconazole. In embodiments, the cell culture medium supplement does not include mitomycin. In embodiments, the cell culture medium supplement does not include 17β-estradiol. In embodiments, the cell culture medium supplement does not include chloramphenicol. In embodiments, the cell culture medium supplement does not include voriconazole. In embodiments, the cell culture medium supplement does not include ziprasidoue maleate. In embodiments, the cell culture medium supplement does not include diclofenac sodium. In embodiments, the cell culture medium supplement does not include etomoxir. In embodiments, the cell culture medium supplement does not include a statin.

In embodiments, the cell culture medium does not include a hydrophobic drug compound. In embodiments, the cell culture medium supplement does not include a hydrophobic drug compound. In embodiments, the cell culture medium does not include albumin. In embodiments, the cell culture medium does not include a protein. In embodiments, the cell culture medium is serum-free cell culture medium. Thus, the invention includes compositions (e.g., cell culture media and supplements) that are serum-free, albumin-free, or protein-free.

In embodiments, the cell culture medium includes albumin. In embodiments, the cell culture medium includes a protein.

In embodiments, the population of T cells includes T cells that are capable of greater retention of phenotype, greater expansion, greater potency, and/or higher transduction efficiency compared to corresponding T cells in a population of T cells that is in combination with a cell culture medium that does not include a cyclodextrin and at least one lipid.

In an aspect is provided a serum-free cell culture medium composition including linoleic acid, at least one (e.g., about or at least about 1, 2, 3, 4, 5, 6, 7, 8, 9, or 10) other omega-6 fatty acid, cholesterol, and a methylated cyclodextrin.

In an aspect is provided a serum-free cell culture supplement composition, including linoleic acid, at least one (e.g., about or at least about 1, 2, 3, 4, 5, 6, 7, 8, 9, or 10) other omega-6 fatty acid, cholesterol, and a methylated cyclodextrin.

In embodiments, the methylated cyclodextrin is present in a cell culture medium at a level from 50 μM to 200 μM, from 55 μM to 195 μM, from 60 μM to 190 μM, from 65 μM to 185 μM, from 70 μM to 180 μM, from 75 μM to 175 μM, from 80 μM to 170 μM, from 85 μM to 165 μM, from 90 μM to 160 μM, from 95 μM to 155 μM, from 95 μM to 150 μM, from 100 μM to 145 μM, from 105 μM to 140 μM, from 110 μM to 135 μM, from 115 μM to 130 μM, or from 120 μM to 125 μM.

In embodiments, the cell culture medium includes a level of at least one lipid that is at least about 5 μM, 10 μM, 15 μM, 20 μM, 25 μM, 30 μM, 35 μM, 40 μM, 45 μM, or 50 μM. In embodiments, the cell culture medium includes a level of at least one lipid that is from about 5 μM, 10 μM, or 15 μM to about 20 μM, 25 μM, 30 μM, 35 μM, 40 μM, 45 μM, or 50 μM. In embodiments, the cell culture medium includes a level of at least one lipid that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 19 μM to about 21 μM. In embodiments, the level of the at least one lipid in the cell culture medium is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium includes a level of at least one fatty acid that is at least about 5 μM, 10 μM, 15 μM, 20 μM, 25 μM, 30 μM, 35 μM, 40 μM, 45 μM, or 50 μM. In embodiments, the cell culture medium includes a level of at least one fatty acid that is from about 5 μM, 10 μM, or 15 μM to about 20 μM, 25 μM, 30 μM, 35 μM, 40 μM, 45 μM, or 50 μM. In embodiments, the cell culture medium includes a level of at least one fatty acid that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 19 μM to about 21 μM. In some embodiments, the level of the at least one fatty acid in the cell culture medium is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium includes a level of at least one polyunsaturated fatty acid (e.g., an omega-6 fatty acid such as arachidonic acid, linoleic acid, and/or gamma-linolenic acid) that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of at least one polyunsaturated fatty acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of at least one polyunsaturated fatty acid that is from about 0.05 μM to about 50 μM, from about 0.5 μM to about 10 μM, from about 1 μM to about 10 μM, or from about 1 μM to about 5 μM.

In embodiments, the cell culture medium includes a level of arachidonic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of arachidonic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of arachidonic acid that is from about 0.05 μM to about 50 μM, from about 1 μM to about 10 μM, from about 0.5 μM to about 10 μM, or from about 1 μM to about 5 μM.

In embodiments, the cell culture medium includes a level of linoleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linoleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linoleic acid that is from about 0.05 μM to about 50 μM, from about 0.5 μM to about 10 μM, from about 1 μM to about 10 μM, or from about 1 μM to about 5 μM.

In embodiments, the cell culture medium includes a level of linolenic that (e.g., alpha-linolenic acid, gamma-linolenic acid, or a combination thereof) is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linolenic that that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linolenic that that is from about 0.05 μM to about 50 μM, from about 0.5 μM to about 10 μM, from about 5 μM to about 10 μM, or from about 1 μM to about 5 μM.

In embodiments, the cell culture medium includes a level of at least one fatty acid other than a polyunsaturated fatty acid (e.g., a saturated fatty acid such as myristic acid, palmitic acid or stearic acid, and/or a monounsaturated fatty acid such as palmitoleic acid or oleic acid) that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of the fatty acid(s) that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of the fatty acid(s) that is from about 0.05 μM to about 50 μM, from about 0.5 μM to about 10 μM, from about 5 μM to about 10 μM, or from about 1 μM to about 5 μM.

In embodiments, the cell culture medium includes a level of myristic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of myristic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of myristic acid that is from about 0.05 μM to about 50 μM, from about 0.5 μM to about 10 μM, from about 5 μM to about 10 μM, from about 1 μM to about 5 μM.

In embodiments, the cell culture medium includes a level of palmitic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitic acid that is from about 0.05 μM to about 50 μM, from about 0.5 μM to about 10 μM, from about 5 μM to about 10 μM, or from about 1 μM to about 5 μM.

In embodiments, the cell culture medium includes a level of stearic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of stearic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of stearic acid that is from about 0.05 μM to about 50 μM, from about 0.5 μM to about 10 μM, from about 5 μM to about 10 μM, or from about 1 μM to about 5 μM.

In embodiments, the cell culture medium includes a level of palmitoleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitoleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitoleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of oleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of oleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of oleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of cholesterol that is at least about 5 μM, 10 μM, 15 μM, 20 μM, 25 μM, 30 μM, 35 μM, 40 μM, 45 μM, or 50 μM. In embodiments, the cell culture medium includes a level of cholesterol that is from about 5 μM, 10 μM, or 15 μM to about 20 μM, 25 μM, 30 μM, 35 μM, 40 μM, 45 μM, or 50 μM. In embodiments, the cell culture medium includes a level of cholesterol that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 18 μM to about 21 μM, from about 19 μM to about 20 μM. In embodiments, the cell culture medium includes a level of cholesterol that is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the at least one other omega-6 fatty acid is a polyunsaturated omega-6 fatty acid.

In embodiments, the at least one other omega-6 fatty acid is gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid. In embodiments, the at least one other omega-6 fatty acid is gamma linolenic acid. In embodiments, the at least one other omega-6 fatty acid is eicosadienoic acid. In embodiments, the at least one other omega-6 fatty acid is dihomo-gamma-linolenic acid. In embodiments, the at least one other omega-6 fatty acid is arachidonic acid. In embodiments, the at least one other omega-6 fatty acid is docosadienoic acid. In embodiments, the at least one other omega-6 fatty acid is adrenic acid. In embodiments, the at least one other omega-6 fatty acid is docosapentaenoic acid. In embodiments, the at least one other omega-6 fatty acid is tetracosatetraenoic acid. In embodiments, the at least one other omega-6 fatty acid is tetracosapentaenoic acid.

In embodiments, the at least one other omega-6 fatty acid is arachidonic acid.

In embodiments, the serum-free cell culture medium composition further includes alpha-linolenic acid. In embodiments, the serum-free cell culture supplement composition further includes alpha-linolenic acid.

In embodiments, the serum-free cell culture medium composition further includes myristic acid, oleic acid, palmitic acid, palmitoleic acid, and/or stearic acid. In embodiments, the serum-free cell culture medium composition further includes myristic acid. In embodiments, the serum-free cell culture medium composition further includes oleic acid. In embodiments, the serum-free cell culture medium composition further includes palmitic acid. In embodiments, the serum-free cell culture medium composition further includes palmitoleic acid. In embodiments, the serum-free cell culture medium composition further includes stearic acid.

In embodiments, the serum-free cell culture supplement composition further includes myristic acid, oleic acid, palmitic acid, palmitoleic acid, and/or stearic acid. In embodiments, the serum-free cell culture supplement composition further includes myristic acid. In embodiments, the serum-free cell culture supplement composition further includes oleic acid. In embodiments, the serum-free cell culture supplement composition further includes palmitic acid. In embodiments, the serum-free cell culture supplement composition further includes palmitoleic acid. In embodiments, the serum-free cell culture supplement composition further includes stearic acid.

In embodiments, the serum-free cell culture medium composition includes at least 3, 4, 5, 6, 7, or 8 different fatty acids.

In embodiments, the serum-free cell culture supplement composition includes at least 3, 4, 5, 6, 7, or 8 different fatty acids.

In embodiments, the serum-free cell culture medium composition includes 3, 4, 5, 6, 7, or 8 different fatty acids.

In embodiments, the serum-free cell culture supplement composition includes 3, 4, 5, 6, 7, or 8 different fatty acids.

In embodiments, the serum-free cell culture medium composition includes: (a) any one of linolenic acid, arachidonic acid, myristic acid, oleic acid, palmitic acid, palmitoleic acid, or stearic acid; (b) 2, 3, 4, 5, 6, or 7 of linolenic acid, arachidonic acid, myristic acid, oleic acid, palmitic acid, palmitoleic acid, and stearic acid; (c) linolenic acid, arachidonic acid, myristic acid, oleic acid, palmitic acid, palmitoleic acid, and stearic acid; (d) a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid; (e) monounsaturated fatty acid, and the monounsaturated fatty acid is palmitoleic acid, oleic acid, eicosenoic acid, erucic acid, or nervonic acid; (f) polyunsaturated fatty acid, and the polyunsaturated fatty acid is hexadecatrienoic acid, alpha-linolenic acid, gamma-linolenic acid, stearidonic acid, eicosadienoic acid, eicosatrienoic acid, dihomo-gamma-linolenic acid, mead acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid; (g) omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid; (h) omega-6 fatty acid, and the omega-6 fatty acid is linoleic acid, arachidonic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid; or (i) omega-9 fatty acid, and the omega-9 fatty acid is palmitoleic acid, oleic acid, eicosenoic acid, erucic acid, nervonic acid, or mead acid.

In embodiments, the serum-free cell culture supplement composition includes: (a) any one of linolenic acid, arachidonic acid, myristic acid, oleic acid, palmitic acid, palmitoleic acid, or stearic acid; (b) 2, 3, 4, 5, 6, or 7 of linolenic acid, arachidonic acid, myristic acid, oleic acid, palmitic acid, palmitoleic acid, and stearic acid; (c) linolenic acid, arachidonic acid, myristic acid, oleic acid, palmitic acid, palmitoleic acid, and stearic acid; (d) a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid; (e) monounsaturated fatty acid, and the monounsaturated fatty acid is palmitoleic acid, oleic acid, eicosenoic acid, erucic acid, or nervonic acid; (f) polyunsaturated fatty acid, and the polyunsaturated fatty acid is hexadecatrienoic acid, alpha-linolenic acid, gamma-linolenic acid, stearidonic acid, eicosadienoic acid, eicosatrienoic acid, dihomo-gamma-linolenic acid, mead acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid; (g) omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid; (h) omega-6 fatty acid, and the omega-6 fatty acid is linoleic acid, arachidonic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid; or (i) omega-9 fatty acid, and the omega-9 fatty acid is palmitoleic acid, oleic acid, eicosenoic acid, erucic acid, nervonic acid, or mead acid.

In embodiments, the cholesterol is synthetic cholesterol.

In embodiments, the methylated cyclodextrin is a methylated α-cyclodextrin, a methylated 3-cyclodextrin, or a methylated γ-cyclodextrin. In embodiments, the methylated cyclodextrin is a methylated α-cyclodextrin. In embodiments, the methylated cyclodextrin is a methylated β-cyclodextrin. In embodiments, the methylated cyclodextrin is a methylated γ-cyclodextrin. In embodiments, the methylated cyclodextrin is methyl-β-cyclodextrin.

In embodiments, the serum-free cell culture medium composition further includes an unmethylated cyclodextrin. In embodiments, the serum-free cell culture supplement composition further includes an unmethylated cyclodextrin.

In embodiments, the molar ratio of the methylated cyclodextrin to TCOL is less than 10.5:1, less than 10:1, less than 9.5:1, less than 9:1, less than 8.5:1, less than 8:1, less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, is less than 0.5:1, less than 0.25:1, or less than 0.1:1.

In embodiments, the molar ratio of the methylated cyclodextrin to TL in the composition is less than 11.5:1, less than 11:1, less than 10.5:1, less than 10:1, less than 9.5:1, less than 9:1, less than 8.5:1, less than 8:1, less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1.

In embodiments, the serum-free cell culture medium does include or does not include albumin. In embodiments, the serum-free cell culture supplement does include or does not include albumin. Thus, the invention includes serum-free media and supplements that are be albumin free. When albumin is present, it may be recombinant human serum-albumin (rHSA).

In embodiments, the cell culture medium supplement includes albumin. In embodiments, the cell culture medium supplement includes a protein.

In embodiments, the serum-free cell culture medium does not include a protein. In embodiments, the serum-free cell culture supplement does not include a protein.

In one embodiment, a cell culture medium supplement including 0.00647 mol/L of synthetic cholesterol, 0.0676 mol/L of methyl-β-cyclodextrin, 0.00072 mol/L of arachidonic acid, 0.00508 mol/L of linoleic acid, 0.00014 mol/L of linolenic acid, 27.75422 mol/L of hot water, and 27.75422 mol/L of cold water is provided. In embodiments, an effective dilution of the cell culture medium supplement is from about 1:10 to about 1:5000 (e.g., about 1:10, about 1:20, about 1:30, about 1:40, about 1:50, about 1:60, about 1:70, about 1:80, about 1:90, about 1:100, about 1:150, about 1:200, about 1:250, about 1:300, about 1:350, about 1:400, about 1:450, about 1:455, about 1:460, about 1:465, about 1:470, about 1:475, about 1:480, about 1:485, about 1:490, about 1:495, about 1:500, about 1:505, about 1:510, about 1:515, about 1:520, about 1:525, about 1:530, about 1:535, about 1:540, about 1:545, about 1:550, about 1:555, about 1:560, about 1:565, about 1:570, about 1:575, about 1:580, about 1:585, about 1:590, about 1:595, about 1:600, about 1:650, about 1:700, about 1:750, about 1:800, about 1:850, about 1:900, about 1:950, about 1:1000, about 1:1500, about 1:2000, about 1:2500, about 1:3000, about 1:3500, about 1:4000, about 1:4500, about 1:5000). In embodiments, an effective dilution of the cell culture medium supplement is from about 1:250 to about 1:750. In embodiments, an effective dilution of the cell culture medium supplement is from about 1:400 to about 1:600.

In one embodiment, a cell culture medium supplement including 0.00647 mol/L of synthetic cholesterol, 0.0676 mol/L of methyl-β-cyclodextrin, 0.0000657 mol/L of arachidonic acid, 0.00036 mol/L of linoleic acid, 0.00036 mol/L of linolenic acid, 0.00044 mol/L of myristic acid, 0.00035 mol/L of oleic acid, 0.00039 mol/L of palmitic acid, 0.00039 mol/L of palmitoleic acid, 0.00035 mol/L of stearic acid, 27.75422 mol/L of hot water, and 27.75422 mol/L of cold water is provided. In embodiments, an effective dilution of the cell culture medium supplement is from about 1:10 to about 1:5000 (e.g., about 1:10, about 1:20, about 1:30, about 1:40, about 1:50, about 1:60, about 1:70, about 1:80, about 1:90, about 1:100, about 1:150, about 1:200, about 1:250, about 1:300, about 1:350, about 1:400, about 1:450, about 1:455, about 1:460, about 1:465, about 1:470, about 1:475, about 1:480, about 1:485, about 1:490, about 1:495, about 1:500, about 1:505, about 1:510, about 1:515, about 1:520, about 1:525, about 1:530, about 1:535, about 1:540, about 1:545, about 1:550, about 1:555, about 1:560, about 1:565, about 1:570, about 1:575, about 1:580, about 1:585, about 1:590, about 1:595, about 1:600, about 1:650, about 1:700, about 1:750, about 1:800, about 1:850, about 1:900, about 1:950, about 1:1000, about 1:1500, about 1:2000, about 1:2500, about 1:3000, about 1:3500, about 1:4000, about 1:4500, about 1:5000). In embodiments, an effective dilution of the cell culture medium supplement is from about 1:250 to about 1:750. In embodiments, an effective dilution of the cell culture medium supplement is from about 1:400 to about 1:600.

In one embodiment, a cell culture medium supplement including 0.00647 mol/L of synthetic cholesterol, 0.0676 mol/L of methyl-3-cyclodextrin, 0.00032 mol/L of arachidonic acid, 0.00105 mol/L of linoleic acid, 0.00105 mol/L of linolenic acid, 0.00011 mol/L of myristic acid, 0.00273 mol/L of oleic acid, 0.0017 mol/L of palmitic acid, 0.00017 mol/L of palmitoleic acid, 0.00202 mol/L of stearic acid, 27.75422 mol/L of hot water, and 27.75422 mol/L of cold water is provided. In embodiments, an effective dilution of the cell culture medium supplement is from about 1:10 to about 1:5000 (e.g., about 1:10, about 1:20, about 1:30, about 1:40, about 1:50, about 1:60, about 1:70, about 1:80, about 1:90, about 1:100, about 1:150, about 1:200, about 1:250, about 1:300, about 1:350, about 1:400, about 1:450, about 1:455, about 1:460, about 1:465, about 1:470, about 1:475, about 1:480, about 1:485, about 1:490, about 1:495, about 1:500, about 1:505, about 1:510, about 1:515, about 1:520, about 1:525, about 1:530, about 1:535, about 1:540, about 1:545, about 1:550, about 1:555, about 1:560, about 1:565, about 1:570, about 1:575, about 1:580, about 1:585, about 1:590, about 1:595, about 1:600, about 1:650, about 1:700, about 1:750, about 1:800, about 1:850, about 1:900, about 1:950, about 1:1000, about 1:1500, about 1:2000, about 1:2500, about 1:3000, about 1:3500, about 1:4000, about 1:4500, about 1:5000). In embodiments, an effective dilution of the cell culture medium supplement is from about 1:250 to about 1:750. In embodiments, an effective dilution of the cell culture medium supplement is from about 1:400 to about 1:600.

### Combinations and Methods of Culturing

The invention also includes compositions of different compounds, as well as methods for preparing and/or using such compositions. These different compounds may be present (1) as or in a culture medium supplement or (2) a culture medium. Further, such combinations may be present in kits. Compositions of the invention may include (1) cholesterol and/or a derivative thereof, (2) one or more cyclodextrin, (3) one or more fatty acid, (4) 2-DG, and/or (5) one other compound (e.g., a prostaglandin, Etomoxir, a leukotriene, a statin, etc.). Thus, compositions of the invention include culture media that contain cholesterol, one or more fatty acid and 2-DG but not cyclodextrin. Compositions of the invention also include culture media that contain cholesterol, one or more fatty acid and cyclodextrin but not 2-DG. Further, compositions of the invention also include culture media that contain cholesterol, one or more fatty acid, cyclodextrin and 2-DG.

Compositions set out herein include culture medium supplements, additives to culture medium supplements, and culture media. Of course, the amount of a particular compounds present will vary with the type of composition. As an example, assuming a 10× culture medium supplement is prepared by mixing a 5× solution with another solution to arrive at a 1× concentration. Further assume that the 5× solution contains 500 μg/ml of Compound A. In such an instance, would contain culture medium supplement 100 μg/ml of Compound A and the culture medium would contain 10 μg/ml of Compound A.

Ultimately, formulations of the invention will typically be designed for the preparation of culture media. Further, such culture media will generally be formulated to have desired characteristics. A number of such desired characteristics are set out elsewhere herein. Further, such desired characteristics include high level cell expansion rate and selective expansion of one cell type over another cell type (e.g., CD4+ T cells over CD8+ T cells). Thus, the amount of various compounds present will typically be adjusted to further the desired purpose to be achieved by culturing the cells.

In an aspect is provided a combination including: (i) a population of T cells, (ii) a cell culture medium that comprises a cyclodextrin and at least one lipid, and/or (iii) 2-DG.

In the context of a combination comprising a population of T cells and a cell culture medium, the “cell culture medium” does not include compounds that may originate from the T cell population. Thus, a cell culture medium that comprises a cyclodextrin and at least one lipid comprises the cyclodextrin and the at least one lipid at the time the cell culture medium is combined with the population of T cells.

In embodiments, culturing (e.g., expanding) T cells comprises activating (e.g., stimulating) the T cells. In embodiments, a population of T cells does not increase, or increases little (e.g., less than about 10%) unless the T cells are activated. Various methods and compositions for stimulating T cells are known in the art. In embodiments, the T cells are activated by contacting (e.g., adding to medium comprising the T cells) the T cells with an antibody, ligand [such as phytohemagglutinin (PHA)], or a chemical compound [such as 12-O-Tetradecanoylphorbol-13-acetate (TPA) or ionomycin]. In embodiments, T cells are activated (e.g., stimulated) with anti-CD3/CD28 antibody coated beads (such as Dynabeads®), PHA, a soluble anti-CD3 antibody, or a plate-bound anti-CD3 antibody. In embodiments, T cells are stimulated with T cell-activating antibodies that are coupled (e.g., covalently attached to) or absorbed onto beads. In embodiments, the beads are superparamagnetic spherical polymer particles. In embodiments, the particles have a uniform size.

In embodiments, the at least one lipid is cholesterol, a fatty acid, a fatty acid ester, a phospholipid, or a glycerolipid. In embodiments, the at least one lipid is cholesterol. In embodiments, the at least one lipid is a fatty acid. In embodiments, the at least one lipid is a fatty acid ester. In embodiments, the at least one lipid is a phospholipid. In embodiments, the at least one lipid is a glycerolipid.

In embodiments, the fatty acid is a saturated fatty acid, a monounsaturated fatty acid, or a polyunsaturated fatty acid. In embodiments, the fatty acid is a saturated fatty acid. In embodiments, the fatty acid is a monounsaturated fatty acid. In embodiments, the fatty acid is a polyunsaturated fatty acid.

In embodiments, the fatty acid is a monounsaturated fatty acid. In embodiments, the fatty acid is a polyunsaturated fatty acid. In some embodiments, the fatty acid is one or more fatty acid type selected from the group consisting of (1) a saturated fatty acid, (2) a monounsaturated fatty acid, and (1) a polyunsaturated fatty acid, as well as combinations of such fatty acids (e.g., two polyunsaturated fatty acids, three monounsaturated fatty acid, and one saturated fatty acid).

In embodiments, the fatty acid is an omega-3 fatty acid, an omega-6 fatty acid, or an omega-9 fatty acid, or a combination of one or more (e.g., two or more or three) of an omega-3 fatty acid, an omega-6 fatty acid, or an omega-9 fatty acid. In embodiments, the fatty acid is a LC-PUFA. In embodiments, the fatty acid is a saturated fatty acid. In embodiments, the fatty acid is a monounsaturated fatty acid. In embodiments, the fatty acid is a polyunsaturated fatty acid. In embodiments, the fatty acid is linoleic acid.

In embodiments, the combination further includes linolenic acid. In embodiments, the linolenic acid is alpha-linolenic acid, gamma-linolenic acid, or alpha-linolenic acid and gamma-linolenic acid. In embodiments, the linolenic acid is alpha-linolenic acid. In embodiments, the linolenic acid is gamma-linolenic acid. In embodiments, the linolenic acid is alpha-linolenic acid and gamma-linolenic acid.

In embodiments, the combination further includes arachidonic acid. In embodiments, the combination further includes myristic acid, oleic acid, palmitic acid, palmitoleic acid, and/or stearic acid. In embodiments, the combination further includes myristic acid. In embodiments, the combination further includes oleic acid. In embodiments, the combination further includes palmitic acid. In embodiments, the combination further includes palmitoleic acid. In embodiments, the combination further includes stearic acid.

In embodiments, the at least one lipid is: (a) any one of linoleic acid, linolenic acid, arachidonic acid, myristic acid, oleic acid, palmitic acid, palmitoleic acid, or stearic acid; (b) 2, 3, 4, 5, 6, or 7 of linoleic acid, linolenic acid, arachidonic acid, myristic acid, oleic acid, palmitic acid, palmitoleic acid, and stearic acid; (c) linoleic acid, linolenic acid, arachidonic acid, myristic acid, oleic acid, palmitic acid, palmitoleic acid, and stearic acid; (d) a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid; (e) monounsaturated fatty acid, and the monounsaturated fatty acid is palmitoleic acid, oleic acid, eicosenoic acid, erucic acid, or nervonic acid; (f) polyunsaturated fatty acid, and the polyunsaturated fatty acid is hexadecatrienoic acid, linoleic acid, alpha-linolenic acid, gamma-linolenic acid, stearidonic acid, eicosadienoic acid, eicosatrienoic acid, dihomo-gamma-linolenic acid, mead acid, arachidonic acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid; (g) omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid; (h) omega-6 fatty acid, and the omega-6 fatty acid is linoleic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid; or (i) omega-9 fatty acid, and the omega-9 fatty acid is palmitoleic acid, oleic acid, eicosenoic acid, erucic acid, nervonic acid, or mead acid. In embodiments, the at least one lipid is any one of linoleic acid, linolenic acid, arachidonic acid, myristic acid, oleic acid, palmitic acid, palmitoleic acid, or stearic acid. In embodiments, the at least one lipid is 2, 3, 4, 5, 6, or 7 of linoleic acid, linolenic acid, arachidonic acid, myristic acid, oleic acid, palmitic acid, palmitoleic acid, and stearic acid. In embodiments, the at least one lipid is linoleic acid, linolenic acid, arachidonic acid, myristic acid, oleic acid, palmitic acid, palmitoleic acid, and stearic acid. In embodiments, the at least one lipid is a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid. In embodiments, the at least one lipid is monounsaturated fatty acid, and the monounsaturated fatty acid is palmitoleic acid, oleic acid, eicosenoic acid, erucic acid, or nervonic acid. In embodiments, the at least one lipid is polyunsaturated fatty acid, and the polyunsaturated fatty acid is hexadecatrienoic acid, linoleic acid, alpha-linolenic acid, gamma-linolenic acid, stearidonic acid, eicosadienoic acid, eicosatrienoic acid, dihomo-gamma-linolenic acid, mead acid, arachidonic acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid. In embodiments, the at least one lipid is omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid. In embodiments, the at least one lipid is omega-6 fatty acid, and the omega-6 fatty acid is linoleic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid. In embodiments, the at least one lipid is omega-9 fatty acid, and the omega-9 fatty acid is palmitoleic acid, oleic acid, eicosenoic acid, erucic acid, nervonic acid, or mead acid.

In embodiments, the at least one lipid is cholesterol. In embodiments, the cholesterol is synthetic cholesterol.

In embodiments, the cyclodextrin is an α-cyclodextrin, a β-cyclodextrin, or a γ-cyclodextrin. In embodiments, the cyclodextrin is an α-cyclodextrin. In embodiments, the cyclodextrin is a β-cyclodextrin.

In embodiments, the cyclodextrin is a γ-cyclodextrin. In embodiments, the cyclodextrin is methylated. In embodiments, the cyclodextrin is methyl-β-cyclodextrin.

In embodiments, the cyclodextrin is one or more of the following: 2-hydroxypropyl-β-cyclodextrin, sulfobutylether-β-cyclodextrin, 2-hydroxypropyl-γ-cyclodextrin, 2,6-dimethyl-α-cyclodextrin, hydroxypropyl-γ-cyclodextrin, hydroxyethyl-β-cyclodextrin, β-cyclodextrin polysulfate, trimethyl β-cyclodextrin, and/or γ-cyclodextrin polysulfate. In embodiments, the cyclodextrin is 2-hydroxypropyl-β-cyclodextrin, sulfobutylether-β-cyclodextrin, 2-hydroxypropyl-γ-cyclodextrin, 2,6-dimethyl-α-cyclodextrin, hydroxypropyl-γ-cyclodextrin, hydroxyethyl-β-cyclodextrin, β-cyclodextrin polysulfate, trimethyl β-cyclodextrin, and/or γ-cyclodextrin polysulfate.

In embodiments, the composition includes a plurality of different cyclodextrins, wherein the plurality of cyclodextrins includes at least two cyclodextrins (e.g., about or at least about 2, 3, 4, 5, 6, 7, 8, 9, or 10 cyclodextrins). In embodiments, the composition includes different cyclodextrins ranging from about two to about ten (e.g., from about two to about ten, from about three to about ten, from about four to about ten, from about five to about ten, from about two to about eight, from about three to about eight, from about four to about eight from about two to about ten, etc.).

In embodiments, the plurality of cyclodextrins includes at least one (e.g., about or at least about 1, 2, 3, 4, 5, 6, 7, 8, 9, or 10) α-cyclodextrin, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 2 α-cyclodextrins, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 3 α-cyclodextrins, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 4 α-cyclodextrins, at least one (e.g., about or at least about 1, 2, 3, 4, 5, 6, 7, 8, 9, or 10) β-cyclodextrin, and/or at least one (e.g., about or at least about 1, 2, 3, 4, 5, 6, 7, 8, 9, or 10) γ-cyclodextrin. In embodiments, the plurality of cyclodextrins includes at least 5 α-cyclodextrins, at least one (e.g., about or at least about 1, 2, 3, 4, 5, 6, 7, 8, 9, or 10) O-cyclodextrin, and/or at least one (e.g., about or at least about 1, 2, 3, 4, 5, 6, 7, 8, 9, or 10) γ-cyclodextrin.

In embodiments, the combination includes linoleic acid, cholesterol, and the cyclodextrin.

In embodiments, the combination further comprises 2-DG. In embodiments, the combination includes a level of 2-DG that is about 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 3 mM, 4 mM, 5 mM, 6 mM, 7 mM, 8 mM, 9 mM, or 10 mM. In embodiments, the cell culture medium includes a level of 2-DG from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.25 mM to about 5 mM, from about 0.25 mM to about 5 mM, from about 0.25 mM to about 4 mM, from about 0.25 mM to about 3 mM, from about 0.25 mM to about 2 mM, or from about 0.25 mM to about 1 mM. In embodiments, the level of 2-DG is less than 5 mM, 4 mM, 3 mM, 2 mM, 1 mM, 0.5 mM, or 0.25 mM. In embodiments, the level of 2-DG is about 5 mM, 4 mM, 3 mM, 2 mM, 1 mM, 0.5 mM, or 0.25 mM.

In embodiments, at least about 5%, 10%, 15%, 20%, 25%, 30%, 35%, 40%, 45%, 50%, 55%, 60%, 65%, 70%, 75%, 80%, 85%, 90%, 98% (e.g., from about 5% to about 50%, from about 5% to about 50%, from about 5% to about 50%, from about 5% to about 98%, from about 5% to about 85%, from about 5% to about 75%, from about 5% to about 60%, from about 10% to about 98%, from about 15% to about 95%, from about 20% to about 95%, from about 40% to about 80%, from about 65% to about 99%, etc.) of the cholesterol molecules in a composition or combination are within (e.g., at least a portion thereof is inside of) the ring of a cyclodextrin molecule.

In embodiments, at least about 5%, 10%, 15%, 20%, 25%, 30%, 35%, 40%, 45%, 50%, 55%, 60%, 65%, 70%, 75%, 80%, 85%, 90%, 98% (e.g., from about 5% to about 50%, from about 5% to about 50%, from about 5% to about 50%, from about 5% to about 98%, from about 5% to about 85%, from about 5% to about 75%, from about 5% to about 60%, from about 10% to about 98%, from about 15% to about 95%, from about 20% to about 95%, from about 40% to about 80%, from about 65% to about 99%, etc.) of the fatty acid molecules in a composition or combination are within (e.g., at least a portion thereof is inside of) the ring of a cyclodextrin molecule.

In embodiments, the combination includes a cyclodextrin and cholesterol, wherein the molar ratio of TC to TCOL is less than 10.5:1, less than 10:1, less than 9.5:1, less than 9:1, less than 8.5:1, less than 8:1, less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1. The molar ratio of TC to the TCOL may thus be from about 10.5:1 to about 0.1:1, from about 8:1 to about 0.1:1, from about 7:1 to about 0.1:1, from about 6:1 to about 0.1:1, from about 5:1 to about 0.1:1, from about 3:1 to about 0.1:1, from about 2:1 to about 0.1:1, etc.

In embodiments, the combination includes a cyclodextrin and at least one fatty acid, wherein the molar ratio of TC to TFA is less than 11.5:1, less than 11:1, less than 10.5:1, less than 10:1, less than 9.5:1, less than 9:1, less than 8.5:1, less than 8:1, less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1. The molar ratio of the cyclodextrin to the at least one fatty acid may thus be from about 11.5:1 to about 0.1:1, from about 10.5:1 to about 0.1:1, from about 8:1 to about 0.1:1, from about 7:1 to about 0.1:1, from about 6:1 to about 0.1:1, from about 5:1 to about 0.1:1, from about 3:1 to about 0.1:1, from about 2:1 to about 0.1:1, etc.

In embodiments, combination includes a cyclodextrin, cholesterol, and at least one fatty acid, wherein the molar ratio of (1) TC to (2) TCOL and TFA (e.g., the sum of the molar values of TCOL and TFA) is less than 7.5:1, less than 7:1, less than 6.5:1, less than 6:1, less than 5.5:1, less than 5:1, less than 4.5:1, less than 4:1, less than 3.5:1, less than 3:1, less than 2.5:1, less than 2:1, less than 1.5:1, less than 1:1, less than 0.5:1, less than 0.25:1, or less than 0.1:1. The molar ratio of (1) TC to (2) the TCOL and TFA may thus be from about 7:1 to about 0.1:1, from about 6:1 to about 0.1:1, from about 5:1 to about 0.1:1, from about 3:1 to about 0.1:1, from about 2:1 to about 0.1:1, etc.

In embodiments, the of TC to TCOL on a molar basis is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of TFA to TC on a molar basis is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of TFA on a molar basis to TCOL is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of total polyunsaturated fatty acid molecules on a molar basis to other fatty acid molecules is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of omega-3 polyunsaturated fatty acid molecules to other fatty acid molecules is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of omega-6 polyunsaturated fatty acid molecules to other fatty acid molecules is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the ratio of omega-9 polyunsaturated fatty acid molecules to other fatty acid molecules is 10:90, 15:85, 20:80, 25:75, 30:70, 35:65, 40:60, 45:55, 50:50, 55:45, 60:40, 65:35, 70:30, 75:25, 80:20, 85:15, and 90:10 (e.g., from about 10:90 to about 90:10, from about 20:80 to about 90:10, from about 30:70 to about 90:10, from about 40:60 to about 90:10, from about 10:90 to about 80:20, from about 10:90 to about 70:30, from about 10:90 to about 60:40, from about 20:80 to about 80:20, from about 30:70 to about 70:30, from about 40:60 to about 60:40, etc.).

In embodiments, the combination further includes a prostaglandin, a corticosteroid, a leukotriene, a lipoxin, a protectin, a resolvin, an oligonucleotide, or hydrophobic drug compound. In embodiments, the combination further includes a prostaglandin. In embodiments, the combination further includes a corticosteroid. In embodiments, the combination further includes a leukotriene. In embodiments, the combination further includes a lipoxin. In embodiments, the combination further includes a protectin. In embodiments, the combination further includes a resolvin. In embodiments, the combination further includes an oligonucleotide. In embodiments, the combination further includes hydrophobic drug compound. In embodiments, the hydrophobic drug compound is etomoxir or a statin. In embodiments, the hydrophobic drug compound is etomoxir. In embodiments, the hydrophobic drug compound is a statin.

In embodiments, the cell culture medium includes a level of cyclodextrin that is about 200 μM, 150 μM, 140 μM, 130 μM, 120 μM, 110 μM, 100 μM, 90 μM, 80 μM, 70 μM, 60 μM, 50 μM, 40 μM, 30 μM, 20 μM, 10 μM, 5 μM, 1 μM, 0.5 μM, 0.25 μM, 0.1 μM, 0.05 μM, 0.025 μM, 0.001 μM, less than 200 μM, less than 150 μM, less than 140 μM, less than 130 μM, less than 120 μM, less than 110 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM, less than 1 μM, less than 0.5 μM, less than 0.25 μM, less than 0.1 μM, less than 0.05 μM, less than 0.025 μM, or less than 0.01 μM.

In embodiments, the cell culture medium includes a level of cyclodextrin that is from about 50 μM to about 200 μM, from about 55 μM to about 195 μM, from about 50 μM to about 190 μM, from about 65 μM to about 185 μM, from about 70 μM to about 180 μM, from about 75 μM to about 175 μM, from about 80 μM to about 170 μM, from about 85 μM to about 165 μM, from about 90 μM to about 160 μM, from about 90 μM to about 155 μM, from about 95 μM to about 150 μM, from about 100 μM to about 145 μM, from about 105 μM to about 140 μM, from about 110 μM to about 135 μM, from about 115 μM to about 130 μM, or from about 120 μM to about 125 μM.

In embodiments, the cell culture medium includes a level of at least one lipid that is at least about 5 μM, 10 μM, 15 μM, 20 μM, 25 μM, 30 μM, 35 μM, 40 μM, 45 μM, or 50 μM. In embodiments, the cell culture medium includes a level of at least one lipid that is from about 5 μM, 10 μM, or 15 μM to about 20 μM, 25 μM, 30 μM, 35 μM, 40 μM, 45 μM, or 50 μM. In embodiments, the cell culture medium includes a level of at least one lipid that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 19 μM to about 21 μM. In embodiments, the level of the at least one lipid in the cell culture medium is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium includes a level of at least one fatty acid that is at least about 5 μM, 10 μM, 15 μM, 20 μM, 25 μM, 30 μM, 35 μM, 40 μM, 45 μM, or 50 μM. In embodiments, the cell culture medium includes a level of at least one fatty acid that is from about 5 μM, 10 μM, or 15 μM to about 20 μM, 25 μM, 30 μM, 35 μM, 40 μM, 45 μM, or 50 μM. In embodiments, the cell culture medium includes a level of at least one fatty acid that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 19 μM to about 21 μM. In some embodiments, the level of the at least one fatty acid in the cell culture medium is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the cell culture medium includes a level of at least one polyunsaturated fatty acid (e.g., an omega-6 fatty acid such as arachidonic acid, linoleic acid, and/or gamma-linolenic acid) that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of at least one polyunsaturated fatty acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of at least one polyunsaturated fatty acid that is from about 0.05 μM to about 50 μM, from about 0.5 μM to about 10 μM, from about 5 μM to about 10 μM, or from about 1 μM to about 5 μM.

In embodiments, the cell culture medium includes a level of arachidonic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of arachidonic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of arachidonic acid that is from about 0.05 μM to about 50 μM, from about 0.5 μM to about 10 μM, from about 5 μM to about 10 μM, or from about 1 μM to about 5 μM.

In embodiments, the cell culture medium includes a level of linoleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linoleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linoleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of linolenic that (e.g., alpha-linolenic acid, gamma-linolenic acid, or a combination thereof) is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linolenic that that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of linolenic that that is from about 0.05 μM to about 50 μM, from about 0.5 μM to about 10 μM, from about 5 μM to about 10 μM, or from about 1 μM to about 5 μM.

In embodiments, the cell culture medium includes a level of at least one fatty acid other than a polyunsaturated fatty acid (e.g., a saturated fatty acid such as myristic acid, palmitic acid or stearic acid, and/or a monounsaturated fatty acid such as palmitoleic acid or oleic acid) that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of the fatty acid(s) that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of the fatty acid(s) that is from about 0.05 μM to about 50 μM, from about 0.5 μM to about 10 μM, from about 5 μM to about 10 μM, or from about 1 μM to about 5 μM.

In embodiments, the cell culture medium includes a level of myristic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of myristic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of myristic acid that is from about 0.05 μM to about 50 μM, from about 0.5 μM to about 10 μM, from about 5 μM to about 10 μM, from about 1 μM to about 5 μM.

In embodiments, the cell culture medium includes a level of palmitic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitic acid that is from about 0.05 μM to about 50 μM, from about 0.5 μM to about 10 μM, from about 5 μM to about 10 μM, or from about 1 μM to about 5 μM.

In embodiments, the cell culture medium includes a level of stearic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of stearic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of stearic acid that is from about 0.05 μM to about 50 μM, from about 0.5 μM to about 10 μM, from about 5 μM to about 10 μM, or from about 1 μM to about 5 μM.

In embodiments, the cell culture medium includes a level of palmitoleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitoleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of palmitoleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of oleic acid that is at least about 0.05 μM, 0.1 μM, 0.5 μM, 1 μM, 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of oleic acid that is from about 0.05 μM, 0.1 μM, 0.5 μM, or 1 μM to about 1.5 μM, 2 μM, 2.5 μM, 3 μM, 3.5 μM, 4 μM, 4.5 μM, 5 μM, 10 μM, 15 μM, 20 μM, 25 μM, 30 μM, 35 μM, 40 μM, 45 μM, or 50 μM. In embodiments, the cell culture medium includes a level of oleic acid that is from about 0.05 μM to about 50 μM, from about 0.5 μM to about 10 μM, from about 5 μM to about 10 μM, or from about 1 μM to about 5 μM.

In embodiments, the cell culture medium includes a level of cholesterol (e.g., synthesitic cholortesterol, animal origin free cholesterol, etc.) that is at least about 5 μM, 10 μM, 15 μM, 20 μM, 25 μM, 30 μM, 35 μM, 40 μM, 45 μM, or 50 μM. In embodiments, the cell culture medium includes a level of cholesterol that is from about 5 μM, 10 μM, or 15 μM to about 20 μM, 25 μM, 30 μM, 35 μM, 40 μM, 45 μM, or 50 μM. In embodiments, the cell culture medium includes a level of cholesterol that is from about 5 μM to about 50 μM, from about 5 μM to about 40 μM, from about 5 μM to about 30 μM, from about 10 μM to about 30 μM, from about 11 μM to about 29 μM, from about 12 μM to about 28 μM, from about 13 μM to about 27 μM, from about 14 μM to about 26 μM, from about 15 μM to about 25 μM, from about 16 μM to about 24 μM, from about 17 μM to about 23 μM, from about 18 μM to about 22 μM, from about 18 μM to about 21 μM, from about 19 μM to about 20 μM. In embodiments, the cell culture medium includes a level of cholesterol that is 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, or 30 μM.

In embodiments, the combination does not include a drug compound. In embodiments, the combination does not include alprostadil, cefotiam hexetil HCl, benexate HCl, dexamethasone, iodine, nicotine, nimesulide, nitroglycerin, omeprazol, PGE2, piroxicam, tiaprofenic acid, cisapride, hydrocortisone, ludomethacin, itraconazole, mitomycin, 17β-estradiol, chloramphenicol, voriconazole, ziprasidoue maleate, diclofenac sodium, etomoxir or a statin. In embodiments, the combination does not include a hydrophobic drug compound.

In embodiments, the cell culture medium does not include albumin. In embodiments, the cell culture medium does not include a protein. In embodiments, the cell culture medium is serum-free cell culture medium.

In embodiments, the cell culture medium includes albumin. In embodiments, the cell culture medium includes a protein.

In embodiments, the population of T cells includes T cells that are capable of greater retention of phenotype, greater expansion, greater potency, and/or higher transduction efficiency compared to corresponding T cells in a population of T cells that is in combination with a cell culture medium that does not include a cyclodextrin and at least one lipid.

In an aspect is provided a method for culturing a T cell population, including incubating the population in a cell culture medium including a cyclodextrin and at least one (e.g., 1, 2, 3, 4, 5, 6, 7, 8, 9, or 10) lipid. In embodiments, the method for culturing a T cell population includes incubating the population in a cell culture medium including (1) a cyclodextrin and (2) at least 2 lipids, least 3 lipids, at least 4 lipids, at least 5 lipids, at least 6 lipids, at least 7 lipids, at least 8 lipids, at least 9 lipids, at least 10 lipids, at least 15 lipids, or at least 20 lipids (e.g., from about 3 to about 20, from about 4 to about 20, from about 5 to about 20, from about 6 to about 20, from about 7 to about 20, from about 3 to about 15, from about 3 to about 12, from about 3 to about 10, from about 3 to about 8, from about 5 to about 20, from about 5 to about 15, from about 5 to about 12, from about 5 to about 9, etc. fatty acids).

In embodiments, the cell culture medium includes the serum-free cell culture supplement composition as provided herein including embodiments thereof.

In embodiments, the T cell population includes CD8+ T cells. In embodiments, the T cell population includes CD4+ T cells. In embodiments, the T cell population includes CD8+ T cells and CD4+ T cells.

In an aspect is provided a method of culturing a T cell population that includes CD8+ T cells and CD4+ T cells while minimizing a change in the ratio of CD8+ T cells to CD4+ T cells within the population, the method includes incubating the population in a medium including a cyclodextrin and a polyunsaturated fatty acid. In embodiments, the polyunsaturated fatty acid is an omega-6 polyunsaturated fatty acid. In embodiments, the omega-6 polyunsaturated fatty acid is linoleic acid.

In embodiments, the medium further includes cholesterol. In embodiments, the medium further includes linolenic acid. In embodiments, the polyunsaturated fatty acid is linolenic acid. In embodiments, the medium further includes arachidonic acid.

In embodiments of the method, minimizing a change in the ratio of CD8+ T cells to CD4+ T cells includes maintaining a ratio of CD8+ T cells to CD4+ T cells in which the number of CD8+ T cells to CD4+ T cells differs by less than 25%, 20%, 15%, 10%, or 5% compared to the number of CD8+ T cells to CD4+ T cells when the population is first contacted with the medium.

In embodiments, when the population is first contacted with the medium, then the population includes a ratio of CD8+ T cells to CD4+ T cells of about 1:1 (e.g., 1.1:1, 1.2:1, 1.3:1, 1.4:1, 1.5:1, 1:1.1, 1:1.2, 1:1.3, 1:1.4, 1:1.5, or about 1.01:1 to 1.1:1 or about 1:1.01 to 1:1.1). In embodiments, when the population is first contacted with the medium, then the population includes a ratio of CD8+ T cells to CD4+ T cells of 1:1.

In embodiments, the medium includes (i) a cyclodextrin; (ii) cholesterol; and (iii) fatty acids, wherein the fatty acids consist of linoleic acid, linolenic acid, and arachidonic acid. In embodiments, the medium lacks any one of, or any combination of, myristic acid, oleic acid, palmitic acid, palmitoleic acid, and stearic acid.

In embodiments, the medium includes a molar ratio of a polyunsaturated fatty acid(s) to other fatty acids of at least 1:1 (e.g., 2:1, 3:1, 4:1, 5:1, 6:1, 7:1, 8:1, 9:1, or 10:1). In embodiments, the medium includes a molar ratio of an omega-3 polyunsaturated fatty acid(s) to other fatty acids of at least 1:1 (e.g., 2:1, 3:1, 4:1, 5:1, 6:1, 7:1, 8:1, 9:1, or 10:1). In embodiments, the medium includes a molar ratio of an omega-6 polyunsaturated fatty acid(s) to other fatty acids of at least 1:1 (e.g., 2:1, 3:1, 4:1, 5:1, 6:1, 7:1, 8:1, 9:1, or 10:1). In embodiments, the medium includes a molar ratio of an omega-9 polyunsaturated fatty acid(s) to other fatty acids of at least 1:1 (e.g., 2:1, 3:1, 4:1, 5:1, 6:1, 7:1, 8:1, 9:1, or 10:1).

In embodiments, the medium includes a molar ratio of a linoleic acid, linolenic acid, and/or arachidonic acid to other fatty acids of at least 1:1 (e.g., 2:1, 3:1, 4:1, 5:1, 6:1, 7:1, 8:1, 9:1, or 10:1). In embodiments, the medium includes a molar ratio of (1) linoleic acid, linolenic acid, and/or arachidonic acid to (2) other fatty acids of at least 2:1, at least 3:1, at least 4:1, at least 5:1, at least 6:1, at least 7:1, at least 8:1, at least 9:1, or at least 10:1.

In embodiments, the method further includes incubating the population for a sufficient period of time until the T cells have reached a desired number, stage of differentiation, and/or phenotype; and optionally harvesting T cells from the culture.

In an aspect is provided a method for preferentially expanding members of a T cell subpopulation, the method including exposing a mixed population of T cells to: (i) cyclodextrin; and (ii) fatty acids, wherein the molar ratio of two or more fatty acids is adjusted to induce the members of the T cell subpopulation to preferentially expand over members of other T cell subpopulations.

In embodiments, the T cell subpopulation is CD8+ T cells.

In embodiments, the mixed population of T cells is exposed to more polyunsaturated fatty acids than other fatty acids. In embodiments, the mixed population of T cells is exposed to more omega-6 polyunsaturated fatty acids than other fatty acids.

In embodiments, the T cell subpopulation is CD4+ T cells. In embodiments, the T cells are primary T cells. In embodiments, the T cells have been isolated from the blood of a human subject.

In embodiments, the T cells are genetically modified T cells. In embodiments, the T cells express a genetically modified T cell receptor. In embodiments, the T cells express a chimeric antigen receptor.

In embodiments, the T cells are T regulatory cells (Tregs), T helper cells, Th17 cells, Th9 cells, T memory cells, T effector memory cells, T central memory cells, terminally differentiated effector (TTD) T cells, naïve T cells, or engineered T cells.

In embodiments, the size of the T cell population doubles at least 3 (e.g., 4, 5, 6, 7, 8, 9, 10) times within 7 days.

In embodiments, the size of the T cell population doubles at least 3, 4, or 5 times within 10 days.

In embodiments, at least 75%, 80%, 85%, 90%, or 95% of the T cells in the T cell population are viable 7, 8, 9, or 10 days after the T cell population is first contacted with the medium. In embodiments, at least 95% of the T cells in the T cell population are viable 10 days after the T cell population is first contacted with the medium.

In embodiments, the method further includes preparing the cultured T cells for administration to a subject suffering from or at risk of suffering from a disease or condition. In embodiments, the method further includes administering the T cells to the subject.

In embodiments is provided a cell culture plate, flask, bag, biofermentor, or bioreactor system (or other culture vessel that is suitable for the culture of T cells) including a combination as provided herein including embodiments thereof.

### Methods for Obtaining Desired CD4+:CD8+ T Cell Ratios

Also included herein are compositions and methods for obtaining T cell populations containing desired CD4+:CD8+ T cell ratios. For example, the present subject matter provides methods for preferentially expanding members of a T cell subpopulation (e.g., CD8+ T cells) within a T cell population. Such methods include contacting a mixed population of T cells (that includes CD8+ T cells, and e.g., CD4+ T cells) with 2-DG.

In an aspect, provided herein are methods of culturing a T cell population that comprises CD8+ T cells and CD4+ T cells while increasing the ratio of CD8+ T cells to CD4+ T cells within the population. In embodiments, the population is incubated in a cell culture medium comprising 2-DG. In embodiments, the cell culture medium further comprises a cyclodextrin and at least one lipid (such as cholesterol and/or a fatty acid). In embodiments, a cyclodextrin and/or lipid (such as cholesterol and/or a fatty acid) is present in an amount or molar ratio disclosed with respect to a cell culture medium, cell culture supplement, or combination provided herein. In embodiments, the cyclodextrin is any cyclodextrin or combination of cyclodextrins disclosed herein. In embodiments, the lipid is any lipid (such as a fatty acid and/or cholesterol) or combination of lipids disclosed herein.

In embodiments, the 2-DG is present at a level from about 0.1 mM to about 5 mM. In embodiments, the combination includes a level of 2-DG that is about 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 3 mM, 4 mM, 5 mM, 6 mM, 7 mM, 8 mM, 9 mM, or 10 mM. In embodiments, the cell culture medium includes a level of 2-DG from about 0.1 mM to about 10 mM, from about 0.1 mM to about 5 mM, from about 0.1 mM to about 4 mM, from about 0.1 mM to about 3 mM, from about 0.1 mM to about 2 mM, from about 0.1 mM to about 1 mM, from about 0.25 mM to about 5 mM, from about 0.25 mM to about 5 mM, from about 0.25 mM to about 4 mM, from about 0.25 mM to about 3 mM, from about 0.25 mM to about 2 mM, or from about 0.25 mM to about 1 mM. In embodiments, the level of 2-DG is less than 5 mM, 4 mM, 3 mM, 2 mM, 1 mM, 0.5 mM, or 0.25 mM. In embodiments, the level of 2-DG is about 5 mM, 4 mM, 3 mM, 2 mM, 1 mM, 0.5 mM, or 0.25 mM.

In embodiments, the cell culture medium comprises serum. In embodiments, serum is human serum. In embodiments, the serum is bovine serum. In embodiments, the bovine serum is fetal bovine serum. In embodiments, the serum is 1%, 2%, 3%, 4%, 5%, 6%, 7%, 8%, 9%, 10%, 11%, 2%1, 13%, 14%, 15%, 16%, 17%, 18%, 19%, 20%, 21%, 22%, 23%, 24%, 25%, 1-5%, 1-10%, 1-15%, 1-20%, 10-20%, 5-15%, 10-20%, or 15-10% of the cell culture medium by volume.

In embodiments, the cell culture medium is a serum-free cell culture medium.

In embodiments, the ratio of CD8+ T cells to CD4+ T cells in the population increases by at least 10%, 20%, 30%, 40%, 50%, 60%, 70%, 80%, 90%, 100%, 2-fold, 2.5-fold, 3-fold, 3.5-fold, 4-fold, 4.5-fold, or 5-fold within about 2, 3, 4, 5, 6, or 7 days after the population is first contacted with the medium.

In embodiments, there are more CD4+ T cells than CD8+ T cells in the population when the population is first contacted with the medium.

In embodiments, the ratio of CD4+ T cells to CD8+ T cells is at least 5:1 (e.g., at least 5:1, 6:1, 7:1, 8:1, 9:1, or 10:1) in the population when the population is first contacted with the medium.

In embodiments, the T cells are primary T cells. In embodiments, the T cells have been isolated from the blood of a human subject. In embodiments, the T cells are genetically modified T cells. In embodiments, the T cells express a genetically modified T cell receptor. In embodiments, the T cells express a chimeric antigen receptor.

In embodiments, the T cells are T regulatory cells (Tregs), T helper cells, Th17 cells, Th9 cells, T memory cells, T effector memory cells, T central memory cells, terminally differentiated effector (TTD) T cells, naïve T cells, or engineered T cells.

In embodiments, the size of the T cell population doubles at least 3 (e.g., 4, 5, 6, 7, 8, 9, 10) times within 7 days.

In embodiments, the size of the T cell population doubles at least 3, 4, or 5 times within 10 days.

In embodiments, at least 75%, 80%, 85%, 90%, or 95% of the T cells in the T cell population are viable 7, 8, 9, or 10 days after the T cell population is first contacted with the medium. In embodiments, at least 95% of the T cells in the T cell population are viable 10 days after the T cell population is first contacted with the medium.

### Treatment Methods

In an aspect is provided a method of treating a disease in a subject in need thereof, the method including administering to the subject T cells obtained by the method provided herein including embodiments thereof. Non-limiting examples of uses for CD8+ T cells (e.g., expanded populations of T cells comprising increased CD8+ T cell proportions, or CD8+ T cells isolated from such expanded populations) include: immunotherapies based on virus-specific T cells such as for cytomegalovirus (CMV) infection and for Epstein-Barr virus (EBV) infection for treatment of immunosuppressed transplant patients. See, e.g., Heslop et al. (2010) Blood 115(5):925-35, the entire content of which is incorporated herein by reference. Additional non-limiting examples include the use of CAR-T and other modes of engineering virus-specific T cells for treatment of cancer and infectious disease. See, e.g., Pule et al. (2008) Nature Medicine 115(5):925-35 and Ghazi et al. (2013) J Immunother 35(2): 159-168, the entire contents of each of which are incorporated herein by reference. Non-limiting examples of uses for CD4+ T cells (e.g., expanded populations of T cells comprising increased CD4+ T cell proportions, or CD4+ T cells isolated from such expanded populations), include the treatment of HIV+ patients, and expanded CD4+ T helper subsets (e.g., TH1, TH2, TH3, TH17, TH9, or TFH), and Regulatory T cells (Treg: CD4+ CD25+FoxP3+) for treating autoimmunity. See, e.g., Tebas et al. (2014) N Engl J Med 370(10):901-10 and Riley et al. (2009) Immunity 30(5): 656-665, the entire contents of each of which are incorporated herein by reference.

In embodiments, the disease is a hyperproliferative disorder. In embodiments, the disease is an autoimmune disease. In embodiments, the disease is an inflammatory disease. In embodiments, the disease is an allergic disease. In embodiments, the disease is an infectious disease.

In embodiments, the infectious disease is a viral infection. In embodiments, the viral infection is a cytomegalovirus infection, a Epstein-Barr virus infection, or a human immunodeficiency virus infection.

In embodiments, the subject has a suppressed immune system. In embodiments, the subject has received a tissue or organ transplant. In embodiments, the subject has acquired immune deficiency syndrome.

In embodiments, the T cells are CD8+ T cells. In embodiments, the T cells are CD4+ T cells.

T cell subpopulations produced using the compositions and methods provided herein can be used in any number of physiological conditions, diseases and/or disease states for therapeutic purposes and/or research/discovery purposes. In embodiments, a condition or disease typified by an aberrant immune response is an autoimmune disease, for example diabetes, multiple sclerosis, myasthenia gravis, neuritis, lupus, rheumatoid arthritis, psoriasis, or inflammatory bowel disease. In embodiments, a condition in which immune suppression would be advantageous include conditions in which a normal or an activated immune response is disadvantageous to the mammal, e.g., allo-transplantation of, e.g., body fluids or parts, to avoid rejection, or in fertility treatments in which inappropriate immune responses have been implicated in failure to conceive and miscarriage. In embodiments, the use of such cells before, during, or after transplantation avoids extensive chronic graft versus host disease which may occur in patients being treated (e.g., transplant patients). In embodiments, the cells may be expanded immediately after harvest or stored (e.g., by freezing) prior to expansion or after expansion and prior to their therapeutic use. In embodiments, such therapies may be conducted in conjunction with known immune suppressive therapies.

In embodiments, T cells are isolated based upon the stage of differentiation. T cell populations may be assessed for the stage of differentiation based upon the presence or absence of certain cellular markers or proteins. Markers used to assess the stage of T cell differentiation include: CD3, CD4, CD5, CD8, CD11c, CD14, CD19, CD20, CD25, CD27, CD33, CD34, CD45, CD45RA, CD45RB, CD56, CD62L, CD123, CD127, CD278, CD335, CD11a, CD45RO, CD57, CD58, CD69, CD95, CD103, CD161, CCR7, as well as the transcription factor FOXP3.

In embodiments, once an appropriate T cell population or sub population has been isolated from a patient or animal, genetic or any other appropriate modification or manipulation may optionally be carried out before the resulting T cell population is expanded using the methods and supports of the invention. The manipulation may, for example, take the form of stimulate/re-stimulation of the T cells with anti-CD3 and anti-CD28 antibodies to activate/re-activate them.

In embodiments, it may be desired to administer activated T cells to a subject and then subsequently redraw blood (or have an apheresis performed), activate and expand T cells therefrom according to a method provided herein, and reinfuse the patient with these activated and expanded T cells. This process can be carried out multiple times every few weeks. In embodiments, T cells can be expanded from blood draws of from 10 ml to 400 ml. In embodiments, T cells are expanded from blood draws of about 20 ml, about 30 ml, about 40 ml, about 50 ml, about 60 ml, about 70 ml, about 80 ml, about 90 ml, or about 100 ml. In embodiments, the administration of the subject compositions may be carried out in any convenient manner, including by aerosol inhalation, injection, ingestion, transfusion, implantation or transplantation. In embodiments, the compositions described herein may be administered to a patient subcutaneously, intradermally, intratumorally, intranodally, intramedullary, intramuscularly, by intravenous (i.v.) injection, or intraperitoneally. In embodiments, T cells are administered to a patient by intradermal or subcutaneous injection. In embodiments, T cells may be administered by i.v. injection. In embodiments, T cells may be injected directly into a tumor, lymph node, or site of infection/inflammation (autoimmunity).

In embodiments, a T cell subpopulation generated according to a method provided herein may have many potential uses, including experimental and therapeutic uses. In embodiments, a small number of T cells are removed from a patient and then manipulated and expanded ex vivo before reinfusing them into the patient. Non-limiting examples of diseases that may be treated in this way are autoimmune diseases and conditions in which suppressed immune activity is desirable (e.g., for allo-transplantation tolerance).

In embodiments, a therapeutic method comprises providing a mammal, obtaining a biological sample from the mammal that contains T cells; expanding/activating the T cells ex vivo in accordance with the methods provided herein; and administering the expanded/activated T cells to the mammal to be treated. In embodiments, the first mammal and the mammal to be treated can be the same or different. In embodiments, the mammal can generally be any mammal, such as a cat, dog, rabbit, horse, pig, cow, goat, sheep, monkey, or human. In embodiments, the first mammal (“donor”) can be syngeneic, allogeneic, or xenogeneic. In embodiments, therapy could be administered to mammals having aberrant immune response (such as autoimmune diseases including, for example diabetes, multiple sclerosis, myasthenia gravis, neuritis, lupus, rheumatoid arthritis, psoriasis, and inflammatory bowel disease), tissue transplantation, or fertility treatments.

In embodiments, T cell subpopulations produced using the compositions and methods provided herein can be used in a variety of applications and treatment modalities. In embodiments, T cell subpopulations can be used in the treatment of disease states including, but not limited to, cancer, autoimmune disease, allergic diseases, inflammatory diseases, infectious diseases, and graft versus host disease (GVHD). In embodiments, a T cell therapy includes infusion to a subject of T cell subpopulations externally expanded by methods provided herein following or not following immune depletion, or infusion to a subject of heterologous externally expanded T cells that have been isolated from a donor subject (e.g., adoptive cell transfer).

Autoimmune diseases or disorders are those diseases that result from an inappropriate and excessive response to a self-antigen. In embodiments, an autoimmune disorder comprises defective Treg cells. Non-limiting examples of autoimmune diseases include: diabetes mellitus, uveoretinitis and multiple sclerosis, Addison's disease, celiac disease, dermatomyositis, Grave's disease, Hashimoto's thyroiditis, alopecia areata, ankylosing spondylitis, autoimmune hepatitis, autoimmune parotitis, hemolytic anemia, pemphigus vulgaris, psoriasis, rheumatic fever, sarcoidosis, scleroderma, spondyloarthropathies, vasculitis, vitiligo, myxedema, pernicious anemia, ulcerative colitis, Crohn's disease, dystrophic epidermolysis bullosa, epididymitis, glomerulonephritis, Graves' disease, Guillain-Barre syndrome, Myasthenia gravis, pernicious anemia, reactive arthritis, rheumatoid arthritis, Sjogren's syndrome, and systemic lupus erthematosus as none limiting examples. In autoimmune disease states, the CD4+ CD25+ T regs may be present in decreased number or be functionally deficient. Tregs from peripheral blood having reduced capacity to suppress T cell proliferation have been found in patients with multiple sclerosis (Viglietta et al., J. Exp. Med. 199:971-979 (2004).), autoimmune polyglandular syndrome type II (Kriegel et al., J. Exp. Med. 199:1285-1291 (2004).), type I diabetes (Lindley et al. Diabetes 54:92-929 (2005).), psoriasis (Sugiyama et al., J. Immunol. 174:164-173 (2005)), and myasthenia gravis (Balandina et al., Blood 105:735-741 (2005)).

In embodiments, treatment of autoimmune disorders with T cell therapy may involve differing mechanisms. In embodiments, blood or another source of immune cells can be removed from a subject inflicted with an autoimmune disorder. In embodiments, a method disclosed herein is used to expand T cell types other than memory T cells from the patient sample. In embodiments, following removal and expansion of autologous cells, inappropriate memory T cells can be depleted within a subject in need thereof by known methods, including low dose total body radiation, thymic irradiation, antithymocyte globulin, and administration of chemotherapy. Examples of chemotherapeutic agents include but are not limited to campath, anti-CD3 antibodies, cytoxin, fludarabine, cyclosporine, FK506, mycophenolic acid, steroids, FR901228, and irradiation. In emodiments, following depletion of the inappropriate memory T cells which are capable of recognizing self-antigens, the externally expanded autologous T cells can be readministered to the subject to reconstitute or restimulate their immune system.

Alternatively, or in addition to the above described treatment modalities, Treg cells can be isolated from sources including peripheral blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. In embodiments, these T cells are expanded using methods provided herein. In embodiments, these expanded Treg cells can be re-administered to a patient to suppress inappropriate immune responses. In embodiments, this Treg therapy may be administered either to suppress the minimal remaining immune responses following immune depletion, or in subjects that have not undergone immune depletion.

In embodiments, a method of treating, reducing the risk of, or the severity of, an adverse GVHD event with T cell therapy is provided. In embodiments, a subject has GVHD. In embodiments, the GVHD follows hematopoietic stem cell transplantation. In embodiments, the GVHD is caused by alloreactive T cells present in the infused hematopoietic stem cell preparation. In embodiments, a subject has received organ transplantation and suffers or is at risk of suffering from graft rejection mediated by alloreactive host T cells. In embodiments, blood or another source of immune cells can be removed from a subject inflicted with GVHD. In embodiments, a method provided herein is used to selectively expand T cell types other than memory T cells, selectively expanding those cell types that do not comprise long-lasting recognition of antigens from the exogenous tissue. In embodiments, following removal and external expansion of autologous cells, inappropriate memory T cells can be depleted within a subject in need thereof by known methods, including low dose total body radiation, thymic irradiation, antithymocyte globulin, and administration of chemotherapy. Examples of chemotherapeutic agents include but are not limited to campath, anti-CD3 antibodies, cytoxin, fludarabine, cyclosporine, FK506, mycophenolic acid, steroids, FR901228, and irradiation. In embodiments, following depletion of the inappropriate memory T cells capable of recognizing antigens on the exogenous tissues, the externally expanded autologous T cells can be readministered to the subject to reconstitute or restimulate their immune system.

In embodiments, Treg cells removed from patient blood can be expanded.

In embodiments, these expanded Treg cells are readministered to a patient to suppress inappropriate immune responses, either to suppress the minimal remaining immune responses following immune depletion, or in subjects that have not undergone immune depletion.

Included herein are methods for treating allergic diseases. In embodiments, an allergic disease comprises T cell dysfunction. Studies have indicated impaired CD4+ CD25+ Treg-mediated inhibition of allergen-specific T helper type 2 (Th2) are present in patients suffering seasonal allergies (Ling E M, et al., Lancet 2004; 363:608-15.; Grindebacke H, et al., Clin Exp Allergy 2004; 34:1364-72.). Furthermore, altered proportions of T cells populations have been implicated in individuals with allergies and asthmatic diseases compared to healthy subjects (Akdis M, et al., J Exp Med 2004; 199:1567-75; Tiemessen M M, et al., J Allergy Clin Immunol 2004; 113:932-9.).

In embodiments, blood can be removed from a subject suffering from an allergic disorder. In embodiments, a method provided herein is used to selectively expand non T memory cell T cell types, selectively expanding those cell types that do not comprise long-lasting recognition of antigens from the inappropriate antigen (e.g., a legume protein). In embodiments, following removal and expansion of autologous cells, inappropriate memory T cells can be depleted within a subject in need thereof by known methods, including low dose total body radiation, thymic irradiation, antithymocyte globulin, and administration of chemotherapy. Examples of chemotherapeutic agents include but are not limited to campath, anti-CD3 antibodies, cytoxin, fludarabine, cyclosporine, FK506, mycophenolic acid, steroids, FR901228, and irradiation. In embodiments, following depletion of the inappropriate memory T cells capable of recognizing antigens on the exogenous tissues, the externally expanded autologous T cells can be readministered to the subject to reconstitute or restimulate their immune system.

In embodiments, Treg cells removed from patient blood can be expanded. These expanded Treg cells can be readministered to a patient to suppress inappropriate immune responses, either to suppress the minimal remaining immune responses following immune depletion, or in subjects that have not undergone immune depletion.

Also provided herein are methods for treating inflammatory diseases and inflammation associated disorders. Many of these diseases can also be categorized as autoimmune disorders. Non-limiting examples of inflammatory diseases and inflammation associated disorders include: diabetes; rheumatoid arthritis; inflammatory bowel disease; familial mediterranean fever; neonatal onset multisystem inflammatory disease; tumor necrosis factor (TNF) receptor-associated periodic syndrome (TRAPS); deficiency of interleukin-1 receptor antagonist (DIRA); and Behcet's disease.

Without being bound by any theory, because of the role of Treg cells in suppressing inappropriate immune responses to non pathogenic antigens, decreased numbers or impaired functioning of these T cell subpopulations can contribute to inflammatory diseases. This is true of, for example, inflammatory bowel disease (M Himmell, et al., Immunology 2012 June; 136(2): 115-122) and rheumatoid arthritis (M Noack, et al., Autoimmunity Reviews 2014 Jun.; 13(6): 668-677).

In embodiments, blood can be removed from a subject suffering from an inflammatory disorder.

In embodiments, a method provided herein can be used to selectively expand non T memory cell T cell types, selectively expanding those cell types that do not comprise long-lasting recognition of inappropriate antigens (e.g., carbamylated proteins in anticarbamylated protein (anti-CarP) antibody mediated rheumatoid arthritis). Following removal and expansion of autologous cells, inappropriate memory T cells can be depleted within a subject in need thereof by known methods, including low dose total body radiation, thymic irradiation, antithymocyte globulin, and administration of chemotherapy. Examples of chemotherapeutic agents include but are not limited to campath, anti-CD3 antibodies, cytoxin, fludarabine, cyclosporine, FK506, mycophenolic acid, steroids, FR901228, and irradiation. In embodiments, following depletion of the inappropriate memory T cells capable of recognizing self-antigens and mounting the resultant inflammatory response, the externally expanded autologous T cells can be readministered to the subject to reconstitute their immune system.

In embodiments, Treg cells removed from patient blood can be expanded.

These expanded Treg cells can be readministered to a patient to suppress inappropriate immune responses, either to suppress the minimal remaining immune responses following immune depletion, or in subjects that have not undergone immune depletion.

Methods for treating hyperproliferative disorders (such as cancer) are also provided herein. In embodiments, increased Treg activity may result in poor immune response to tumor antigens and contribute to immune dysfunction. Elevated populations of CD4+ CD25+ have been found in lung, pancreatic, breast, liver and skin cancer patients, in either the blood or tumor itself (Woo E Y, et al.; J Immunol 2002; 168:4272-6.; Wolf A M, et al. Clin Cancer Res 2003; 9:606-12.; Liyanage U K, et al. J Immunol 2002; 169:2756-61.; Viguier M, et al. J Immunol 2004; 173:1444-53. Ormandy L A, et al. Cancer Res 2005; 65:2457-64.).

In embodiments, T cells specific for tumor antigens or hyperproliferative disorder antigens or antigens associate with a hyperproliferative disorder are expanded using a method or composition disclosed herein. Tumor antigens are proteins that are produced by tumor cells that elicit an immune response, particularly T cell mediate immune responses.

In embodiments, cancers that may be treated include tumors that are not vascularized, or not yet substantially vascularized, as well as vascularized tumors. In embodiments, the cancers may comprise non-solid tumors (such as hematological tumors, for example, leukemias and lymphomas) or may comprise solid tumors. Types of cancers to be treated include but are not limited to carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. Among these are cancers including skin cancer, brain cancer and other central nervous system cancers, head cancer, neck cancer, muscle/sarcoma cancer, bone cancer, lung cancer, esophagus cancer, stomach cancer, pancreas cancer, colon cancer, rectum cancer, uterus cancer, cervix cancer, vagina cancer, vulva cancer, penis cancer, breast cancer, kidney cancer, prostate cancer, bladder cancer, or thyroid cancer or glioblastoma.

Hematologic cancers are cancers of the blood or bone marrow. Non-limiting examples of hematological (or hematogenous) cancers include leukemias, including acute leukemias (such as acute lymphocytic leukemia, acute myelocytic leukemia, acute myelogenous leukemia, and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, and myelodysplasia.

Solid tumors are abnormal masses that usually do not contain cysts or liquid areas. Solid tumors can be benign or malignant. Different types of solid tumors are named for the types of cells that form them (such as sarcomas, carcinomas, and lymphomas). Non-limiting examples of solid tumors such as sarcomas and carcinoma, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In embodiments, expanded T cells are genetically modified the T cells to target antigens expressed on tumor cells through the expression of chimeric antigen receptors (CARs). In embodiments, T cells that express CARs are expanded. CARs are antigen receptors that are designed to recognize cell surface antigens in a human leukocyte antigen independent manner. In embodiments, immune cells may be collected from patient blood or other tissue. In embodiments, the T cells are engineered as described below to express CARs on their surface, allowing them to recognize specific antigens (e.g., tumor antigens). In embodiments, these CAR T cells can then be expanded by methods of the present invention and infused into the patient. In embodiments, T cells are administered at 1×10⁵, 1×10⁶, 1×10⁷, 1×10, 5×10⁸, 1×10⁹, 5×10⁹, 1×10¹⁰, 5×10¹⁰, 1×10¹¹, 5×10¹¹, or 1×10¹² cells to the subject. In embodiments, following patient infusion, the T cells will continue to expand and express the CAR, allowing for the mounting of an immune response against cells harboring the specific antigen the CAR is engineered to recognize.

In embodiments, a cell (e.g., a T cell) engineered to express a CAR, wherein the CAR T cell exhibits an antitumor property, is provided. In embodiments, the CAR is be engineered to comprise an extracellular domain having an antigen binding domain fused to an intracellular signaling domain of the T cell antigen receptor complex zeta chain (e.g., CD3 zeta). In embodiments, the CAR, when expressed in a T cell is able to redirect antigen recognition based on the antigen binding specificity.

In embodiments, the antigen binding moiety of the CAR comprises a target-specific binding element otherwise referred to as an antigen binding moiety. In embodiments, the choice of moiety depends on the type and number of ligands that define the surface of a target cell. For example, the antigen binding domain may be chosen to recognize a ligand that acts as a cell surface marker on target cells associated with a particular disease state. Thus the antigen moiety domain in the CAR may include, e.g., those associated with viral, bacterial and parasitic infections, autoimmune disease and cancer cells.

In embodiments, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. In embodiments, cloning vectors contain transcription and translation terminators, initiation sequences, and promoters useful for regulation of the expression of the desired nucleic acid sequence.

In embodiments, the T cells may be used for nucleic acid immunization and gene therapy, using standard gene delivery protocols. Methods for gene delivery are known in the art. See, e.g., U.S. Pat. Nos. 5,399,346; 5,580,859; 5,589,466. In embodiment, a gene therapy vector is provided.

In embodiments, the nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In embodiments, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

In embodiments, additional promoter elements (e.g., enhancers) regulate the frequency of transcriptional initiation. In embodiments, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Methods of making CAR T cells are known in the art (see, e.g., U.S. Pat. No. 8,906,682).

In embodiment, where a T cell is a CAR T cell, the selection of the antigen binding moiety of the invention may depend on the particular type of cancer to be treated. Tumor antigens are known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RUL RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, surviving and telomerase, prostate-carcinoma tumor antigen-1 (PCTA-1), MAGE, ELF2M, neutrophil elastase, ephrin B2, CD22, insulin growth factor (IGF-1), IGF-II, IGF-I receptor and mesothelin.

In embodiments, the tumor antigen comprises one or more antigenic cancer epitopes associate with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecular such as the oncogene HER-2/Neu/ErbB-2. Yet another group of target antigens are onco-fetal antigens such as carcinoembryonic antigen (CEA). In B-cell lymphoma, the tumor-specific immunoglobulin constitutes a truly tumor-specific immunoglobulin antigen that is unique to the individual tumor. B-cell differentiation antigens such as CD19, CD20; ROR1, CD22, CD23, λ/κ light chains are other candidates for target antigen in B-cell lymphoma.

In embodiments, a tumor antigen is a tumor specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond, or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-1), gp100 (Pmel17), tyrosinanse, TRP-1, TRP-2 and tumor-specific mutilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL. E2A-PRL, H4-RET, IGH-IGK, MYL-RAR; and viral antigens such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-catenin, CDK4, Mum-1, p 15, p 16, 43-9F, 5T4, 791Tgp72, alpha-fetoprotein, beta-HCG, BCA224, BTAA, CA 125, CA 15-3\CA 27/29\BCAA, CA 195, CA 242, CA-50, CAM43, CD68\1, CO-029, FGF-5, C250, GA733\EpCAM, HTgp-175, M344, MA-50, MG7-Ag, MOV18, NB/70K, NY-CO-1, RCAS1, SDCCAG16, TA-90, MAC-2 binding protein\cyclophillin C-associated protein, TAA16, TAG72, TLP, and TPS, CD19, CD20, CD22, ROR1, Mesothelin, CD33/IL3Ra, c-met, PSMA, Glycolipid F77, EGRvIII, GD-2, MY-ESO-1 TCR, MAGE A3 TCR, and others.

Methods of treating infectious diseases are also provided herein. The immune response to infectious diseases involves a balance of anti-pathogen and anti-inflammatory responses. T cells are heavily involved in this intricate balance. In embodiments, infectious pathogens capable of eliciting a T cell response may be bacterial, viral, protozoan, parasitic, or fungal. Treg cells have been implicated in contributing to the chronicity of infection by Helicobacter pylori (Lundgren A, et al. Infect Immun 2003; 71:1755-62.), hepatitis B virus (HBV), and hepatitis C virus (HCV) (Cabrera R, et al., Hepatology 2004; 40:1062-71.; Stoop J N, et al. Hepatology 2005; 41:771-8.; Sugimoto K, et al., Hepatology 2003; 38:1437-48.). In embodiments, an elevation in a particular T cell subpopulation may contribute to the prolonged nature of an infections by inappropriately suppressing memory T cell responses. In embodiments, a composition provided herein is utilized to specifically expand a particular T cell subpopulation and for the treatment of an infectious disease.

In embodiments, an infectious disease is caused by direct contact with a pathogen and spread from person to person, animal to person, or from mother to unborn child.

In embodiments, an infectious diseases is spread through indirect contact, e.g., from contact with an infected surface such as door handle, table, counter or faucet handle. In embodiments, an infectious diseases is spread via insect bites or food contamination. Certain autoimmune disorders, such as HIV or AIDS, and some cancers can increase susceptibility to infectious diseases. Certain treatment regimens that suppress the immune system can also enhance susceptibility to infectious diseases. Example infectious diseases include but are not limited to: smallpox, malaria, tuberculosis, typhus, plague, diphtheria, typhoid, cholera, dysentery, pneumonia.

In embodiments, expansion of T cells may be used in the treatment of infectious disease states. In embodiments, a patient suffering from an infection does not have sufficient immunity to the infectious agent. In embodiments, a method provided herein is used to expand heterologous T memory cells from a donor with immunity to a particular infectious agent and utilized in adoptive T cell transfer. In embodiments, the externally expanded T cells from an infectious agent experienced donor can then be infused into a patient inflicted with the infection. In embodiments, T cells are administered at 1×10⁵, 1×10⁶, 1×10⁷, 1×10⁸, 5×10⁸, 1×10⁹, 5×10⁹, 1×10¹⁰, 5×10¹⁰, 1×10¹¹, 5×10¹¹, or 1×10¹² cells to the subject. In embodiments, the infectious antigen competent donor memory T cells aid in mounting an autologous immune response within the patient.

In embodiments, the treatment of an infectious disease includes the expansion of autologous or heterologous Th17 cells for reinfusion or adoptive cell transfer respectively. In embodiments, T cells can be externally expanded from patient isolated blood or tissue. In embodiments, these expanded T cells can then be infused to the patient to aid in induction of B cells to secrete antibodies against the particular infectious antigen (e.g., Streptococci M-protein, Neisseria pilli, Borrelia burgdorferi lipoprotein VisE, B. pseudomallei polysaccharide antigens, Aspergillus fumigatus galactomannan, or F. tularensis lipopolysaccharide). In embodiments, T cells are administered at 1×10⁵, 1×10⁶, 1×10⁷, 1×10⁸, 5×10⁸, 1×10⁹, 5×10⁹, 1×10¹⁰, 5×10¹⁰, 1×10¹¹, 5×10¹¹, or 1×10¹² cells to the subject.

Existing treatments may be recommended for many of the above listed disease states. T cell subpopulations expanded using methods and compositions disclosed herein may be used as sole replacement therapy in some cases or in conjunction with other known therapies. T cell therapies may be administered prior to, concurrently with, or following administration of other therapies.

In embodiments, a method provided herein is utilized with a vaccine to enhance reactivity of the antigen and enhance in vivo effect. In embodiments, a composition (e.g., comprising T cells) is administered to a patient in conjunction with a composition that enhances T cells in vivo, for example, IL-2, IL-4, IL-7, IL-10, IL-12, and/or IL-15. T cells expanded according to methods provided herein could act as vehicles for gene therapy as described above, by carrying a desired nucleic acid sequence of interest and potentially homing to sites of cancer, disease, or infection. Accordingly, the cells expanded by the methods provided herein may be delivered to a patient in combination with a vaccine, one or more cytokines, one or more therapeutic antibodies, etc. Virtually any therapy that would benefit by a more robust T cell population could be used in conjunction with the compositions provided herein.

### Cellular Production and Vaccines

As indicated elsewhere herein, provided herein are compositions and methods for culturing cells, (e.g., human cells, human diploid cells, primate diploid cells, cells useful for virus production, T cells, etc.) in culture media containing cyclodextrin and one or more lipid. Such cells may be used to produce products such as proteins, nucleic acid molecules, and assembled materials such as viruses and VLPs.

Cell culture (e.g., animal cell culture, such as mammalian cell culture) may be used for the expression of recombinant protein production. Typically, cells such as animal or mammalian cells can express and secrete, or can be genetically engineered to express and secrete, large quantities of a particular protein, more particularly, a glycoprotein of interest, into the culture medium. It will be understood that the glycoprotein produced by a host cell can be endogenous or homologous to the host cell. Alternatively, and usually, the glycoprotein may be heterologous, i.e., foreign, to the host cell, for example, a human glycoprotein may be produced and secreted by, e.g., a Chinese hamster ovary (CHO) host cell. Properly glycosylated, recombinant protein products are increasingly important medically and clinically, as therapeutics and prophylactics products. A desired goal in bioproduction is the development of reliable, economical, and efficient cell culture processes that simultaneously achieve increased final glycoprotein product concentration along with high product quality, which can be determined for e.g., by the sialic acid content of the glycoprotein produced. Thus, provided herein are compositions and methods for the culture of cells and the production of proteins (e.g., recombinant proteins).

Once the cell or the clone for protein production is identified, media or supplement components may need to be adjusted, and additionally, a variety of process parameters may need to be manipulated to increase cell and/or protein titer, and/or to improve the protein quality (glycosylation level). Examples of parameter manipulations may include: the employment of large-scale culture vessels; the alteration of culture conditions such as incubation temperature, dissolved oxygen concentration, pH, temperature shifts, etc. In addition, advances in extended run times can increase the final product concentration while maintaining high protein quality.

Aggregates of the expressed proteins or glycoproteins in the culture media may arise at any stage during the biomanufacturing process. In cell culture, secreted proteins may be exposed to conditions that are unfavorable for protein stability; but more often, the accumulation of high amounts of protein may lead to intracellular aggregation owing to either the interactions of unfolded protein molecules, or due to inefficient recognition of the nascent peptide chain by molecular chaperones responsible for proper folding. Such aggregates can lead to adverse side effects in patients upon administration, thus expensive downstream processing steps are devised to remove the higher molecular weight species. One approach to reduce the level of aggregation is the careful adjustment of critical process parameters and by identifying cell culture additives that disrupt aggregation, for e.g., temperature-shift to 31° C., osmolality above 420 mOsm/kg, agitation at 100 rpm and 0.04% (w/v) antifoam (Biotechnol Bioeng. 2018 May; 115(5):1173-1185).

The presence of cell debris and the contents of dead cells in the culture can negatively impact isolation and/or purification of the protein end-product downstream. Keeping cells viable for a longer periods of time in culture can result in a concomitant reduction in the contamination of the culture medium by cellular proteins and enzymes, e.g., cellular proteases and sialidases, which contribute to the degradation and ultimate reduction in glycoprotein quality. Downstream protein purification concerns may be yet another reason for the maintenance of high cell viability in culture.

Cells culture using compositions and methods provided herein may be used to produce vaccines. Cells useful for virus production include human diploid cells like MRC-5, MRC-5 RCB, WI-38, 2BS, Walvax-2, KMB-17, IMR-90, IMR-91, etc., and non-human diploid cells like VERO (African Green Monkey Kidney), or MDCK (Madin-Darby Canine Kidney). Insect cells, such as sf9 cells, may also be grown using compositions and methods provided herein. Further, insect cells may also be used to produce, for example, recombinant proteins and viruses.

Cell lines suitable for vaccine (e.g., viral production, VLP production, etc.) will generally be of mammalian origin (e.g., Vero cells, horse, cow (e.g., MDBK cells), sheep, dog (e.g., MDCK cells from dog kidneys), cat, and rodent (e.g. hamster cells such as BHK21-F, HKCC cells, or Chinese hamster ovary cells (CHO cells)) but may also be of non-mammalian origin (e.g., chicken cells, insect cells, such as sf9 cells, etc.).

Cell culture medium and/or supplement compositions described above may be used for culturing cells that can be infected with a virus. To infect cells with virus, a suitable medium with or without supplements described above is added to the cells for a few days. Transfection of viral nucleic acid (DNA or RNA) may be carried out on a confluent or near confluent growth of cells, and the transfection medium containing the nucleic acid may be added directly to the cells. In some methods, after several hours for, e.g., 18 hours of incubation at 37° C., the media is changed. In conditions where serum is used for the growth, serum containing media can be added to stop the transfection. About eight days after transfection, the cell supernatants (containing virus) may be collected for harvesting virus, a viral particle, a viral protein or nucleic acid, or a viral fragment.

Before large scale production of cells are prepared for vaccine production, the cells may be tested for: i) virucide; ii) adventitious agents, through the detection of cytopathic effects on the indicator cell lines.

To produce large amounts of product for vaccine production: of virus, a viral particle, a viral protein or nucleic acid, or a viral fragment which forms part of the vaccine, the cells may be expanded on culture dishes, roller bottles, tubes, Roux flasks made of a special glass or plastics, mostly in stationary way, or on microcarrier beads according to manufacturer's instructions. Cells may be cultured semi-continuously (e.g., diploid cells that can be passaged finite times, e.g., HEK (human embryonic kidney) cells, MRC-5, WI-38 cells, or they can be cultured continuously (e.g., transformed cell lines that are immortalized and can be passaged without limit; e.g., HeLa, VERO, Hep-2, LLC-MK2, BGM, etc.).

Semi-continuous cell lines of a finite life are usually diploid and maintain some degree of differentiation. The fact that such cell lines senesce after approximately thirty cycles of division means it is essential to establish a system of master and working banks in order to maintain such lines for long periods.

Continuous cell lines can be propagated indefinitely because they have been transformed into tumor cells. Tumor cell lines are often derived from actual clinical tumors, but transformation may also be induced using viral oncogenes or by chemical treatments. Transformed cell lines present the advantage of almost limitless availability, but the disadvantage of having retained very little of the original in vivo characteristics.

The cells may be expanded under suitable culture conditions for the cell growth and to maintain viability, e.g., at 37° C., 5% CO₂ for most cells. Cells may be inoculated or infected with virus to preferably obtain an MOI of 0.01. In some instances, inoculation may continue without further replacement or media or supplements.

The number of cells present after expansion can be determined using standard counting techniques like using the manual hemocytometer, or by using a cell counter instrument. The viral titer can also be measured by serial dilution in conjunction with the plaque assay. To determine whether the correct clone is obtained for viral titers, the viral nucleic acid may be extracted and sequenced.

Provided herein are methods for growing cells (e.g., human cells, human diploid cells, primate diploid cells, cells useful for virus production, T cells, etc.), that can be infected with virus to produce a vaccine, a virus, a viral particle, a viral protein or nucleic acid, or a viral fragment. Such methods may include methods for culturing a diploid cell population. Steps may include: contacting the cell with the medium comprising cyclodextrin-lipids, or a supplement comprising cyclodextrin-lipids that increases: the growth of the cells, the viable cell density of the cells, the viral titer of a virus infected cells, or a combination thereof. The cells cultured thereby may be used to produce a vaccine, a virus, a viral particle, a viral protein or nucleic acid, or a viral fragment thereof under serum-free conditions. Alternately, cells may be infected with a virus/transfected with nucleic acid derived from a virus.

Virus that is produced from the infected cell described above may be an animal virus, a plant virus or a bacteriophage. These viruses may include, but may not be limited to: Varicella zoster virus (VZV), Rubella, Measles, Mumps, Hepatitis A, Adenovirus, Poliomyelitis, Rotavirus, Rhinovirus, Smallpox, Chickenpox, Yellow fever, Papillomavirus, Ebola virus, HIV, herpesviruses, cytomegalovirus, myxoviruses, paramyxoviruses, enteroviruses, respiratory syncytial virus, Rabies or vesicular stomatitis virus (VSV), and Dengue virus; and/or, the viral particle may be derived from a Parvoviridae family, Retroviridae family, Flaviviridae family, bacteriophage, etc.

The types of cells used for viral transfection, or vaccine, virus, viral particle, viral protein or nucleic acid, viral fragment production may be an animal cell. Animals cell may be a bovine cell, a canine cell, a feline cell, an insect cell, an avian cell, a primate cell or a human cell. Specifically, animal cells may be a diploid cell. Or the cell may be selected from the group consisting of MRC-5, MRC-5 RCB, MRC-9, WI-38, 2BS, Walvax-2, IMR-90, IMR-91, KMB-17, HUT series cell, Chang liver, U937, MDCK, CD4-expressing T cell, CD8-expressing T cell, VERO and any clone of the preceding cells.

Cells may be cultured in a continuous, or a semi-continuous culture. Further, cells may be cultured as an adherent culture or on microcarriers. Each cell type may have specific media and/or basal media requirements. Determination of the appropriate basal media is routinely done in the art, using techniques including but not limited to, metabolic analysis and/or design of experiment (DOE) rationale. A typical method of making a serum-free, cell culture medium for culturing, e.g., a diploid cell, may comprise admixing (i) a basal medium; and either (ii) a supplement that comprises a cyclodextrin and at least one lipid; or (ii) a suitable dilution of the supplements described in Table 1 and/or Table 2. The supplements may further comprises growth factors. Together with the CD (cyclodextrin-lipid) containing supplements described above, a cell can be cultured serum-free for viral transfection, or vaccine, virus, viral particle, viral protein or nucleic acid, viral fragment production.

Provided herein is a system for the supplementation of a cell medium, for culturing for e.g., a diploid cell, may comprise (i) a one or more different cyclodextrins, wherein each cyclodextrin is in a separate vessel; and (ii) two or more different fatty acids, wherein each fatty acid is in a separate vessel.

Provided herein are kits for culturing cells, or cell lines comprising: (i) a population of cells; (ii) a serum-free cell culture medium that comprises a cyclodextrin and at least one lipid. Other kits may comprise (i) a population of cells; (ii) a serum-free basal cell culture medium; and (iii) a supplement that comprises a cyclodextrin and at least one lipid. Yet another kit may comprise: (i) a population of cells; (ii) a serum-free basal cell culture medium; and (iii) a suitable dilution of the supplements as described in Table 1 and/or Table 2. Kits provided herein may be used for the serum-free culture of a cell used for viral transfection, or preparation of a vaccine, virus, viral particle, viral protein or nucleic acid, viral fragment production. These kits may be used to culture an animal cell, wherein the animal cell may be a bovine cell, a feline cell, an insect cell, an avian cell, a primate cell or a human cell, or wherein the animal cell is a diploid cell; or, wherein the cell is selected from the group consisting of MRC-5 cells, MRC-5 RCB cells, MRC-9 cells, WI-38 cells, 2BS cells, Walvax-2 cells, IMR-90 cells, IMR-91 cells, KMB-17 cells, HUT series cells, Chang liver cells, U937 cells, MDCK cells, CD4-expressing T cells, CD8-expressing T cells, VERO cells and any clone of the preceding cells.

Further, cells may be cultivated in a culture medium, washed, then recontacted with culture medium. Viruses, VLPs, and/or viral components may then be obtained from the culture media, either directly or by lysis of the cells. When cells are contacted with viruses or nucleic acid molecules encoding one or more viral components, the cells may be contacted with the virus or nucleic acid nucleic acid molecules at any step, including the wash step or between the wash step and the recontacting with culture medium. Additionally, cells may be used with nucleic acid integrated into their genome encoding the virus or viral components.

Vaccines may be produced by methods such as those set out above or by similar methods where no washing step is required. No washing step will generally be needed when vaccine components are encoded by nucleic acid integrated into the production cell's genome and when nucleic acid (including viruses) can be taken up by cells in culture.

Cell cultured as set out herein may be cultured in a first culture medium before nucleic acid uptake (e.g., by viral inoculation) and a second culture medium at and/or after nucleic acid uptake. Upon uptake of nucleic acid (e.g., by viral infection) by a cell, the cell may be cultured in a second cell culture medium, which may the same as the first culture medium or a different culture medium. For example, in some embodiments, the cell may be cultured in a first cell culture medium, then the first cell culture medium may be removed, the cell may optionally be rinsed (e.g., with an aqueous buffered solution such as PBS), and a second culture medium may be added to the cell. In some embodiments, the second culture medium may contain the nucleic acid (e.g., a virus) for uptake by the cell for uptake by the cell. In some embodiments, the first culture medium and the second culture medium are the same culture medium (e.g., having the same composition, which in some embodiments is not necessarily the same physical medium). In other embodiments, the first culture medium and the second culture medium are different (e.g., having a different composition).

Cells useful for virus production include human diploid cells like MRC-5, MRC-5 RCB, WI-38, 2BS, Walvax-2, KMB-17, IMR-90, IMR-91, etc., and non-human diploid cells like VERO (African Green Monkey Kidney), or MDCK (Madin-Darby Canine Kidney). Insect cells, such as sf9 cells, may also be grown using compositions and methods provided herein. Further, insect cells may also be used to produce viruses.

Viruses that may be produced by methods and by use of compositions set out herein include Varicella zoster virus (VZV), Rubella, Measles, MMR, Hepatitis A, Adenovirus, Poliomyelitis, Rotavirus, Rabies or vesicular stomatitis virus (VSV), and Dengue virus.

Compositions and methods provided herein may also be used to produce virus-like particles (VLPs). Further provided herein are VLPs produced, for example, as set out herein. VLPs may be derived from the Hepatitis B virus and composed of the small HBV derived surface antigen (HBsAg). VLPs may be produced from components of a wide variety of virus families including Parvoviridae (e.g., adeno-associated virus), Retroviridae (e.g., HIV), Flaviviridae (e.g., Hepatitis C virus) and bacteriophages (e.g., QP, AP205).

As noted elsewhere herein, compositions and methods provided herein may also be used to produce vaccines. Vaccine categories that may be produced include inactivated vaccines, attenuated vaccines, toxoid vaccines, subunit vaccines, and conjugate vaccines. Inactivated vaccines are vaccines the disease causing agent, or an agent related thereto, is rendered incapable of disease induction (e.g., by chemical, heat, or radiation treatment). Attenuated vaccine generally contain live or replicable agents (1) for which their virulent properties have been disrupted or (2) that use closely related but less dangerous organisms to produce a broad immune response. Although most attenuated vaccines are viral, some are bacterial in nature. Toxoid vaccines are made from inactivated toxic compounds that cause illness rather than the micro-organism. Not all toxoids are for viruses and microorganisms. For example, Crotalus atrox (i.e., western diamondback rattlesnake) toxoid is used to vaccinate individuals against rattlesnake bites. Subunit vaccines are composed of fragments of an antigen of a disease causing agent that is capable of eliciting a protective immune response. An example of this is the subunit vaccine against Hepatitis B virus that is composed of only surface proteins of this virus.

Cell culture medium and/or supplement compositions provided herein may be suitable for culturing any of the cells described above. These cell culture media and/or supplement compositions will often comprise a cyclodextrin-based lipid compositions. Media composition provided herein may be of at least two varieties: (i) one which comprises cell culture components, a cyclodextrin and at least one lipid, or, (ii) one which comprises a basal cell culture medium, and a separate supplement that comprises cyclodextrin-based lipids. For instance, a cyclodextrin-based lipid supplement may comprise linoleic acid, at least one other omega-6 fatty acid, cholesterol, and a cyclodextrin.

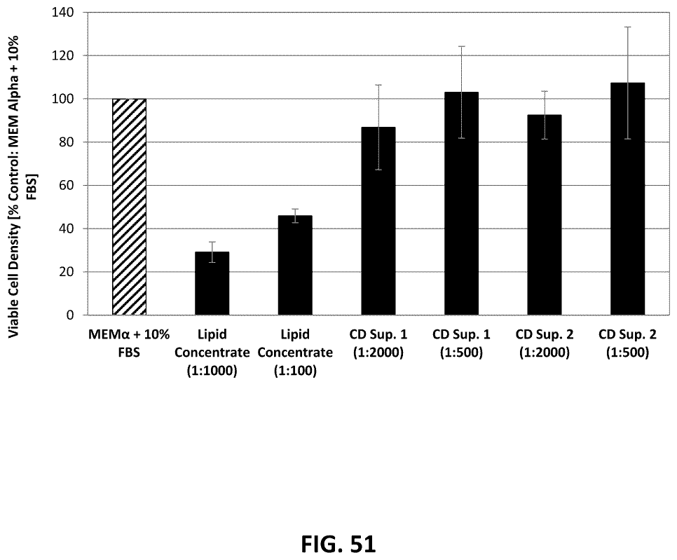

Some exemplary cyclodextrin-based lipid supplements are described in Example 1: Tables 1, 2 and 3 of the instant application, and are sometimes also referred to as CD (cyclodextrin-lipid) supplements 1, 2 and 3 respectively. In addition, a cyclodextrin-lipid containing supplement called Diploid Growth Supplement is commercially available (Thermo Fisher Scientific, Cat. No. A39695SA). Therefore, a medium composition can comprise (i) a suitable basal cell culture medium (such as commercially available (Thermo Fisher Scientific, Cat. No. A39693DK), and (ii) a suitable dilution of the supplements described in Table 1, Table 2, Table 3 (Example 1). A basal medium may contain proteins, vitamins, minerals and amino-acids and can be optionally enriched with fetal calf serum to enable growth. However, in many instances, basal media may be combined with serum-free supplements, such as supplements comprising cyclodextrin-based lipids such as the ones described above (including those described above or in Example 1: Tables 1, 2 and 3, Diploid Growth Supplement (Thermo Fisher Scientific, Cat. No. A39695SA), thus enabling cell growth under serum-free conditions. For optimal growth, the CD supplements or the Diploid Growth Supplement may be diluted appropriately depending on the cell line being cultured. For instance, in a specific example, MRC-5 diploid cells were grown in Diploid Basal SFM (Thermo Fisher Scientific, Cat. No. A39693DK) with CD Supplement 1 or CD Supplement 2 (see Example 1) at a dilution of 1:500 or 1:2000 (also see FIG. 51 and Table 45 of the instant application).

Cell culture supplements used for culturing cells that produce virus or vaccines, etc., may comprise cyclodextrin that is an α-cyclodextrin, a β-cyclodextrin, or a γ-cyclodextrin. In one embodiment, the cyclodextrin may be methylated. In a further embodiment, the cyclodextrin may be a methyl-β-cyclodextrin. In a particular embodiment, the cell culture medium or supplement comprises a level of cyclodextrin that is from about 10 μM to about 200 μM.

Cell culture supplements used for culturing cells that produce virus or vaccines, etc., may comprise cholesterol, wherein the cholesterol may be a synthetic cholesterol, or may be present at a level from about 5 μM to about 30 μM. In one embodiment, the at least one other omega-6 fatty acid is a polyunsaturated omega-6 fatty acid. In a further embodiment, the at least one other omega-6 fatty acid is arachidonic acid. In a particular embodiment, the polyunsaturated omega-6 fatty acid is selected from the group consisting of arachidonic acid, linoleic acid, linolenic acid, myristic acid, oleic acid, palmitic acid, palmitoleic acid and stearic acid.

### Kits and Media Supplement Systems

In an aspect is provided a system for the supplementation of a T cell medium, including: (i) a two or more different cyclodextrins, wherein each cyclodextrin is in a separate vessel; and (ii) two or more different lipids (e.g., fatty acids), wherein each lipid is in a separate vessel. In another aspect is provided a system for the supplementation of a T cell medium, including: (i) a combination of two or more different cyclodextrins, wherein each cyclodextrin is in a separate vessel, and, two or more different lipids (e.g., fatty acids), wherein each lipid is in a separate vessel; and (ii) 2-DG. In yet another aspect is provided a system for the supplementation of a T cell medium, including: 2-DG. In embodiments, the two or more different cyclodextrins include any combination of cyclodextrins disclosed herein. In embodiments, the two or more different lipids include any combination of lipids (e.g., fatty acids) disclosed herein.

In some aspects, kits for culturing T cells comprising a serum free medium, a cyclodextrin, one or more lipids, and/or 2-DG are provided herein.

Kits can also include written instructions for use of the kit, such as instructions for wash steps, culturing conditions and duration of incubation of isolated T cells with compositions provided herein for selective expansion of specific T cell subpopulations.

### Examples of Sources of Mixed Population of T Cells

In embodiments, the starting source for a mixed population of T cell is blood (e.g., circulating blood) which may be isolated from a subject. In embodiments, circulating blood can be obtained from one or more units of blood or from an apheresis or leukapheresis. In embodiments, the apheresis product typically contains lymphocytes, including T cells, monocytes, granulocytes, B cells, other nucleated white blood cells, red blood cells, and platelets. T cells can be obtained from a number of sources, including (but not limited to) blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig.

In embodiments, T cells can be obtained from a unit of blood collected from a subject using any number of techniques known to the skilled artisan, such as Ficoll separation. T cells may be isolated from the circulating blood of a subject. In embodiments, blood may be obtained from the subject by apheresis or leukapheresis. In embodiments, the apheresis product typically contains lymphocytes, including T cells, monocytes, granulocytes, B cells, other nucleated white blood cells, red blood cells, and platelets. In embodiments, prior to exposure to a sensitizing composition and subsequent activation and/or stimulation, a source of T cells is obtained from a subject. In embodiments, the cells collected by apheresis may be washed to remove the plasma fraction and to place the cells in an appropriate buffer or media for subsequent processing steps. In embodiments of the invention, the cells are washed with phosphate buffered saline (PBS). In embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions.

In embodiments, after washing, the cells may be resuspended in a variety of biocompatible buffers, such as, for example, calcium (Ca)-free, magnesium (Mg)-free PBS. In embodiments, the undesirable components of the apheresis sample may be removed and the cells directly resuspended in culture media.

In embodiments, T cells are isolated from peripheral blood lymphocytes by lysing or removing the red blood cells and depleting the monocytes, for example, by centrifugation through a PERCOLL® gradient. In embodiments, a specific subpopulation of T cells can be further isolated by positive or negative selection techniques.

In embodiments, T cells can be positively selected for CD3+ cells. Any selection technique known to one of skill in the art may be used. One non-limiting example is flow cytometric sorting. In another embodiment, T cells can be isolated by incubation with anti-CD3 beads. One non-limiting example is anti-CD3/anti-CD28-conjugated beads, such as DYNABEADS® Human T-Expander CD3/CD28 (Life Technologies Corp., Cat. No. 11141D), for a time period sufficient for positive selection of the desired T cells. In embodiments, the time periods ranges from 30 minutes to 36 hours or longer and all integer values there between. In embodiments, the time period is at least 1, 2, 3, 4, 5, or 6 hours. In another embodiment the time period is 10 to 24 hours. In embodiments, the incubation time period is 24 hours. Longer incubation times, such as 24 hours, can increase cell yield. In embodiments, longer incubation times may be used to isolate T cells in any situation where there are few T cells as compared to other cell types. In embodiments, enrichment of a T cell population by negative selection can be accomplished with a combination of antibodies directed to surface markers unique to the negatively selected cells. One possible method is cell sorting and/or selection via magnetic immunoadherence or flow cytometry that uses a cocktail of monoclonal antibodies direct to cell surface markers present on the cells negatively selected. In embodiments, the fold expansion may differ based on the starting materials due to the variability of donor cells. In embodiments, the normal starting density can be between about 0.5×10⁶ to about 1.5×10⁶.

In embodiments, T cell subpopulations may be generated by selection on the basis of whether one or more marker(s) is/are present or absent. For example, Treg cells may be obtained from a mixed population based upon the selection of cells that are CD4+, CD25+, CD127neg/low and, optionally, FOXP3+. In embodiments, Treg cells may be FOXP3−. Selection, in this instance, effectively refers to “choosing” of the cells based upon one or more definable characteristic. Further, selection can be positive or negative in that it can be for cells have one or more characteristic (positive) or for cells that do not have one or more characteristic (negative).

With respect to Treg cells, for purposes of illustration, these cells may be obtained from a mixed population through the binding of these cells to a surface (e.g., magnetic beads) having attached thereto antibodies that bind to CD4 and/or CD25 and the binding of non-Treg cells to a surface (e.g., magnetic beads) having attached thereto antibodies that binding CD127. As a specific example, magnetic beads having bound thereto an antibody that binds to CD3 may be used to isolate CD3+ cells. Once released, CD3+ cells obtained may then be contacted with magnetic beads having bound thereto an antibody that binds to CD4. The resulting CD3+, CD4+ cells may then be contacted with magnetic beads having bound thereto an antibody that binds to CD25. The resulting CD3+, CD4+, CD25+ cells may then be contacted with magnetic beads having bound thereto an antibody that binds to CD127, where the cells that are collected are those that do not bind to the beads.

In embodiments, multiple characteristics may be used simultaneously to obtain a T cells subpopulation (e.g., Treg cells). For example, a surface containing bound thereto antibodies that bind to two or more cell surface marker may also be used. As a specific example, CD4+, CD25+ cells may be obtained from a mixed population through the binding of these cells to a surface having attached thereto antibodies that bind to CD4 and CD25. The selection for multiple characteristics simultaneously may result in number of undesired cells types “co-purifying” with the desired cell type(s). This is so because, using the specific example above, cells that are CD4+, CD25− and CD4−, CD25+ may be obtained in addition to CD4+, CD25+ cells.

Flow cytometry is particularly useful for the separation of cells based upon desired characteristics. Cells may be separated based upon detectable labels associated with molecules that bind to cells of interested (e.g., a natural ligand such as IL-7 binding to CD127, an antibody specific for CD25, etc.). Thus, ligands that bind to cellular components that may be detected and/or differentiated by flow cytometry systems may be used to purify/isolate T cells that have specific characteristics. Further, the presence or absence of multiple characteristics may be simultaneously determined by flow cytometry.

Included herein are methods for obtaining members of one or more T cell subpopulations, where members of the T cell subpopulations are identified by specific characteristics and separated from cells with differ with respect to these characteristics. Examples of characteristics that may be used in methods of the invention include the presence or absence of the following proteins CD3, CD4, CD5, CD8, CD11c, CD14, CD19, CD20, CD25, CD27, CD33, CD34, CD45, CD45RA, CD56, CD62L, CD123, CD127, CD278, CD335, CCR7, K562P, K562CD19, and FOXP3.

### Examples

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Methodology and Results for Examples 2-4

### Materials and Methods

CD Supplement Formulations: For each 1 L CD Supplement, 0.5 L manufacturing-grade water was pre-cooled at 4° C. and another 0.5 L was heated to ≥80° C. Synthetic cholesterol and powdered fatty acids (myristic, palmitic, and stearic acids—if applicable) were added to the heated water and mixed for a maximum of 10 minutes. While mixing, 36 g methyl-β-cyclodextrin was slowly added, allowing each addition to slightly layer on the surface without touching the circumference of the tank. The tank was covered to maintain the elevated temperature for a minimum of 60 minutes. 54 g methyl-β-cyclodextrin was slowly added as stated previously, covered, and mixed for a minimum of 30 minutes. Mix speed was reduced until foam was dissipated. Pre-cooled water was used to QS and yield the total production volume (1 L) and mixed until temperature reached <25° C. The remaining lipids (arachidonic, linolenic, oleic, and palmitoleic acids—if applicable) were added, covered, and mixed for a minimum of 90 minutes. Each CD Supplement was filter-sterilized using a 1 L STERICUP® Filter Unit (Millipore, Cat. No. SCVPU11RE) and stored at 4° C.

Cell Culture: T cell isolation: De-identified, frozen apheresis bags from normal donors were obtained from HemaCare Corporation. T cells were negatively isolated from PBMCs with the DYNABEADS® UNTOUCHED™ Human T Cells kit (Thermo Fisher Scientific, Cat. No. 11344D).

T cell activation and expansion: T cells (seeding density 1×10⁶ vc/mL) were activated with DYNABEADS® Human T-Expander CD3/CD28 (Thermo Fisher Scientific, Cat. No. 11141D) at a ratio of 3 beads per T cell and cultured in serum-free medium free of cholesterol and free fatty acids supplemented with one of four lipid supplements or no supplement as a control. T cells were counted on days 5, 7, 10, and 12 on a Vi-CELL XR analyzer (Beckman Coulter, Indianapolis IN) and fed to a density of 5×10⁵ vc/mL on days 3, 5, and 7 and 1×10⁶ vc/mL on day 10.

Phenotype: Primary human T cells were expanded for 10 days with one of the CD Supplements or 5% human AB serum. DYNABEADS® were removed from 2×10⁶ cells by magnetic separation. Surface staining was performed with antibodies against CD3 (Invitrogen, Cat. No. CD0329), CD4 (Molecular Probes, Cat. No. A15858), CD8 (Invitrogen, Cat. No. MHCD0828), CCR7 (Molecular Probes, Cat. No. A18370), and CD62L (Thermo Fisher Scientific, Cat. No. MA1-19618). Sequential gating was used to characterize T cells as central memory (TCM: CCR7+/CD62L+), intermediate (CCR7−/CD62L+), and effector memory (TEM: CCR7−/CD62L−). Flow cytometric analysis was performed on a Gallios flow cytometer and Kaluza software (Beckman Coulter, Indianapolis IN).

Cytokine Profiles: Primary human T cells from normal donors were negatively isolated from PBMCs with the DYNABEADS® UNTOUCHED™ Human T Cells kit (Thermo Fisher Scientific, Cat. No. 11344D). T cells (seeding density 1×10⁶ vc/mL) were activated with DYNABEADS® Human T-Expander CD3/CD28 (Thermo Fisher Scientific, Cat. No. 11141D) at a ratio of 3 beads per T cell and cultured in serum-free medium free of cholesterol and free fatty acids supplemented with CD Sup. 1 (1:500) or control medium X-VIVO™ 15 (Lonza, Cat. No. BE02-054Q) supplemented with 5% human AB serum. T cells were counted on days 5 and 7 on a Vi-CELL XR analyzer (Beckman Coulter, Indianapolis IN) and fed to a density of 5×10⁵ vc/mL on days 3, 5, and 7. DYNABEADS® were magnetically removed from the cultures on day 11 and cells were spun to remove conditioned medium and rested overnight in fresh medium. One million T cells were re-stimulated with DYNABEADS® CD3 (Thermo Fisher Scientific, Cat. No. 11151D) at a 1:1 bead to cell ratio and incubated for 24 hours. Supernatants were collected and processed for analysis with the Cytokine Human Magnetic 35-Plex Panel for Luminex™ (Thermo Fisher Scientific, Cat. No. LHC6005M). Analysis was performed using a MAGPIX® system (Luminex Corporation, Austin TX).

### Results

Tables 1-3 summarize the three cyclodextrin-based supplements formulated and tested in cells, for e.g. T cells, diploid cells. Briefly, cholesterol was solubilized in hot water, followed by the slow addition of methyl-β-cyclodextrin and fatty acids. The solutions were monitored until clear in appearance and sterile-filtered. The formulation of CD Sup. 1 (Table 1) was modified while maintaining the same fatty acid:cholesterol mole ratio in CD Sup. 2 and CD Sup. 3 (Tables 2 and 3, respectively). The formulation of CD Sup. 2 is based on the fatty acid concentrations (g/L) in Lipid Concentrate, whereas the formulation of CD Sup. 3 is based on the fatty acid concentrations (g/L) typically found in bovine serum albumin. Chemically Defined Lipid Concentrate (Thermo Fisher Scientific, cat. no. 11905-031), abbreviated “Lipid Concentrate”.

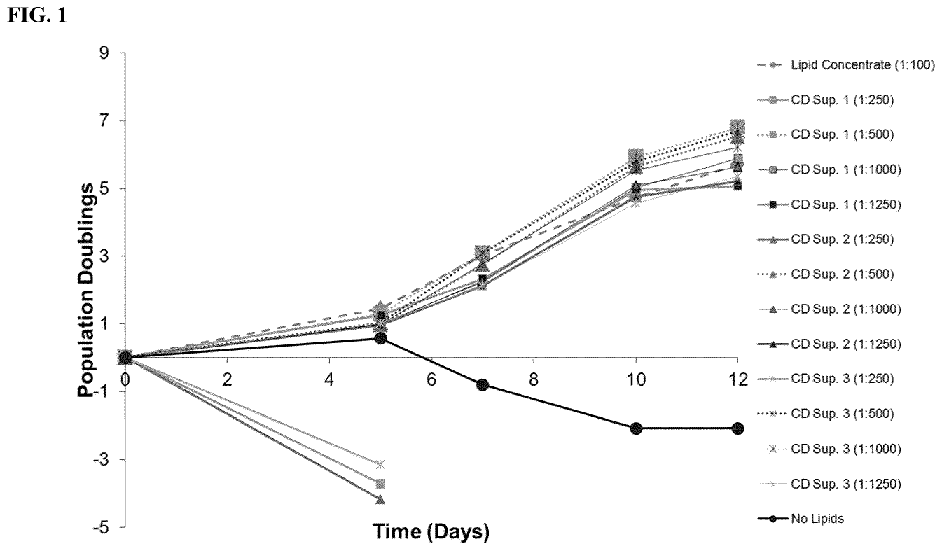

FIGS. 1-6 are a series of graphs demonstrating that serum-free medium containing emulsion-based Lipid Concentrate is suboptimal for human T cell expansion compared to cyclodextrin-based supplementation. Primary human T cells from normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell and cultured in serum-free medium free of cholesterol and free fatty acids supplemented with one of four lipid supplements. T cells were counted on days 5, 7, 10, and 12 on a Beckman-Coulter Vi-CELL XR analyzer and fed to a density of 5×10⁵ vc/mL on days 3, 5, and 7 and 1×10⁶ vc/mL on day 10.

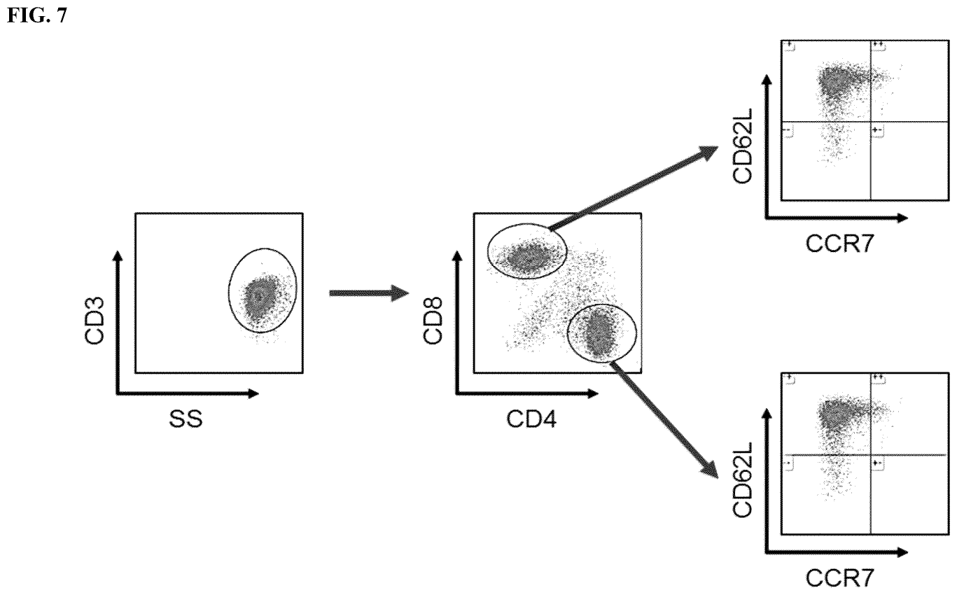

FIGS. 7-10 are a series of graphs demonstrating increased preservation of central memory subsets in T cells cultured with CD Supplements 1, 2, and 3 compared to T cells cultured in medium containing 5% human AB serum.

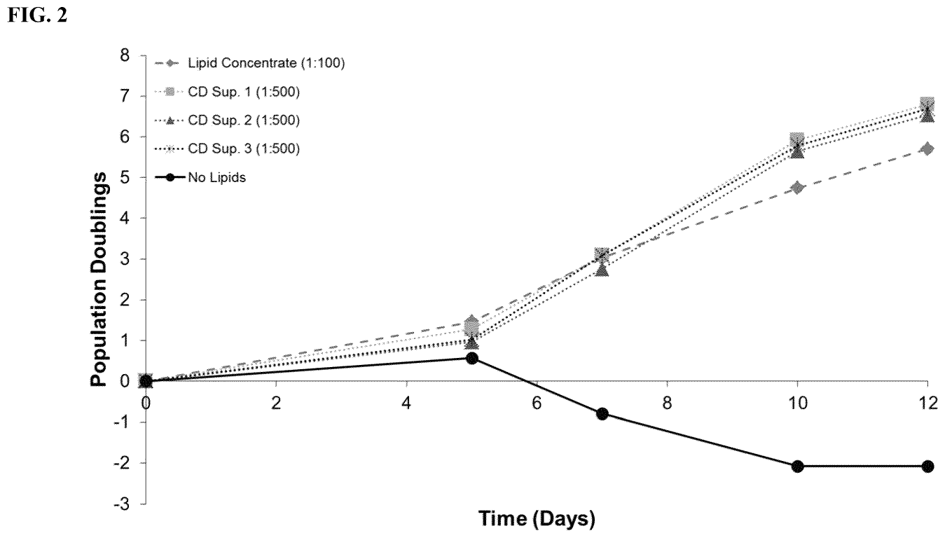

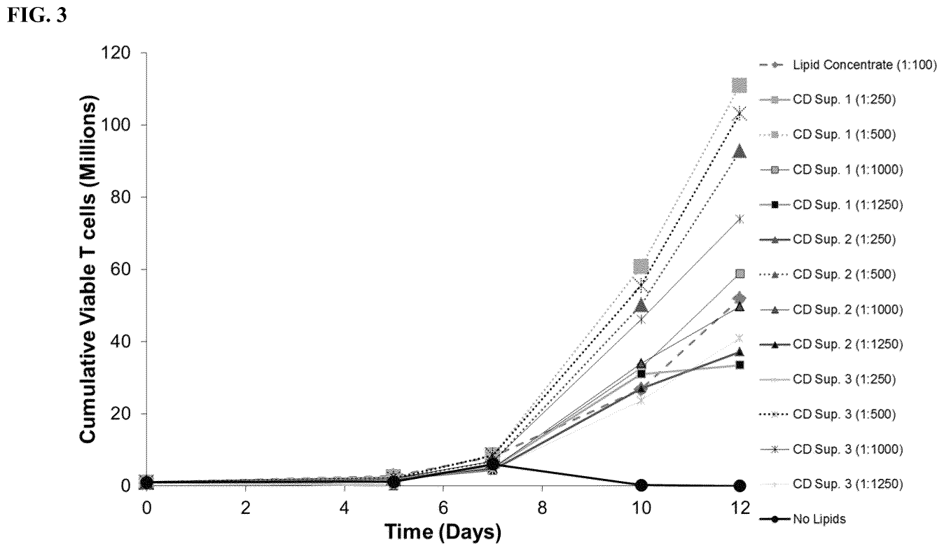

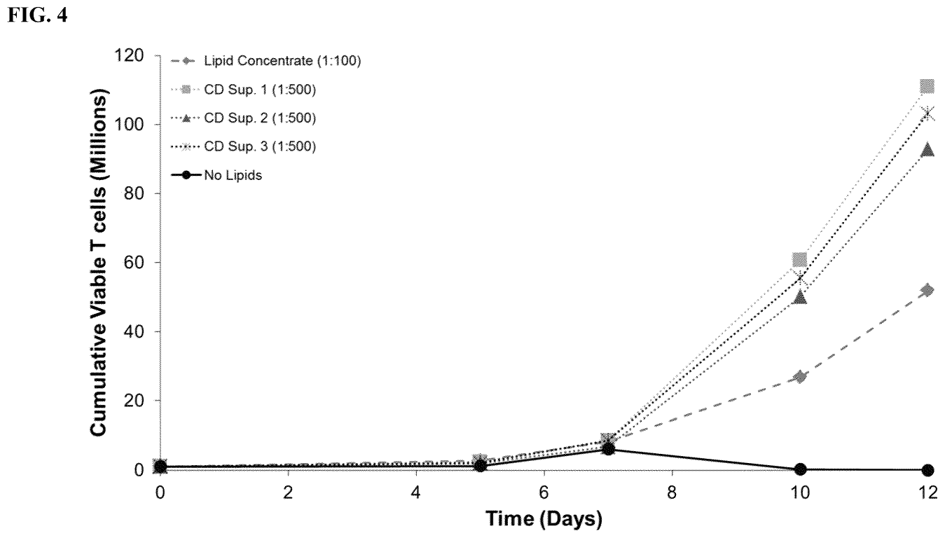

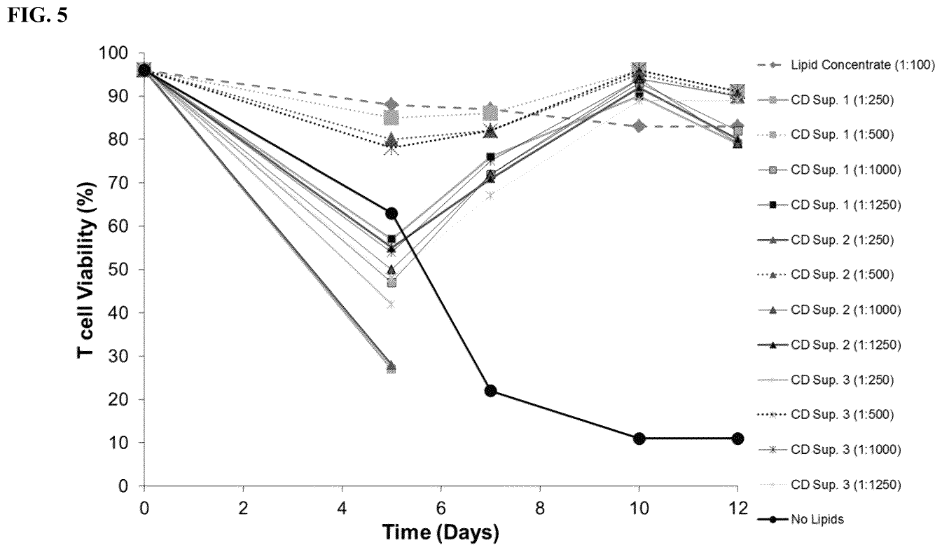

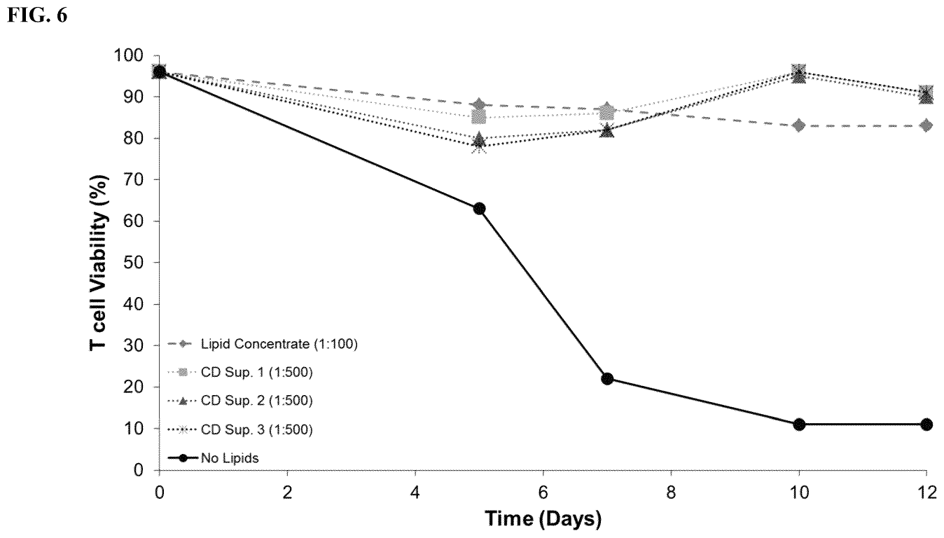

Cell Growth and Viability: T cell expansion is expressed as cumulative population doublings or cumulative viable T cells overtime (days). Results are representative of at least 3 independent experiments. Tables 4-9 quantify the graphical results displayed in FIGS. 1-6. Results demonstrate that Lipid Concentrate (1:100) provides suboptimal T cell expansion (expressed as cumulative population doublings) compared to cyclodextrin-based supplementation via CD Supplements 1, 2, and 3 at selected concentrations in serum-free medium (FIGS. 1 and 2). FIGS. 3 and 4 show the same growth data as FIGS. 1 and 2 but represented in cumulative viable T cells over time (days). The difference in performance among CD Supplements 1, 2, and 3 (1:500) is evident when cell expansion is expressed as cumulative cell number compared to cumulative population doublings by day 12. FIGS. 5 and 6 depict that CD Supplements 1, 2, and 3 (1:500) maintain increased T cell viability compared to Lipid Concentrate, no lipid supplementation, and other concentrations of CD Supplements 1, 2, and 3.

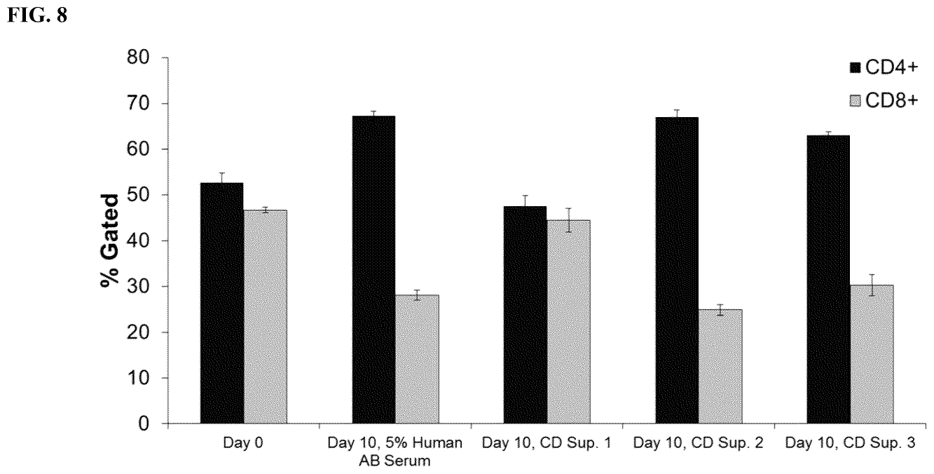

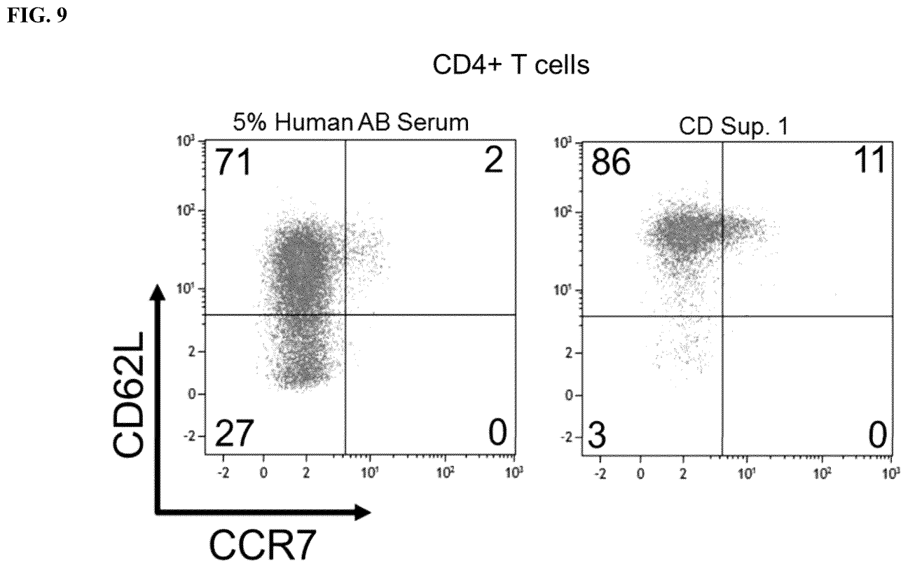

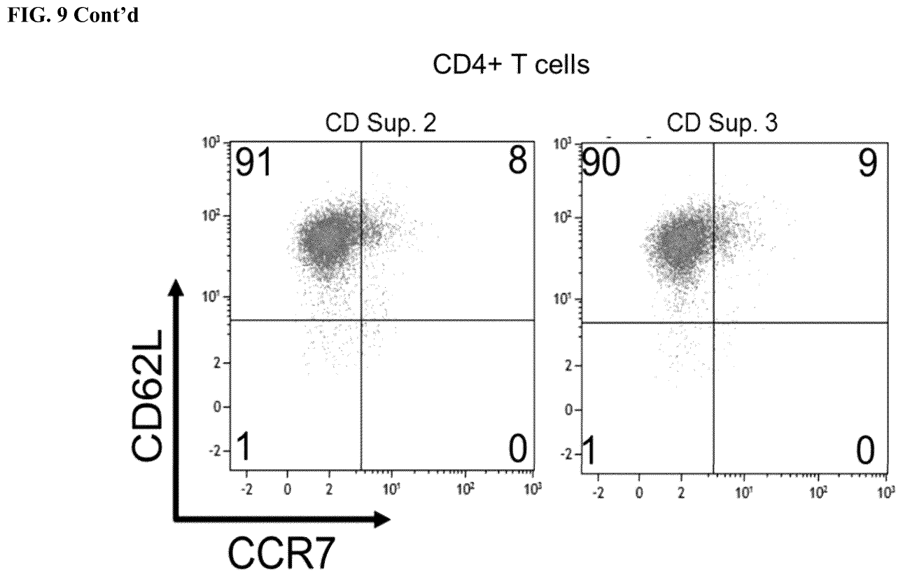

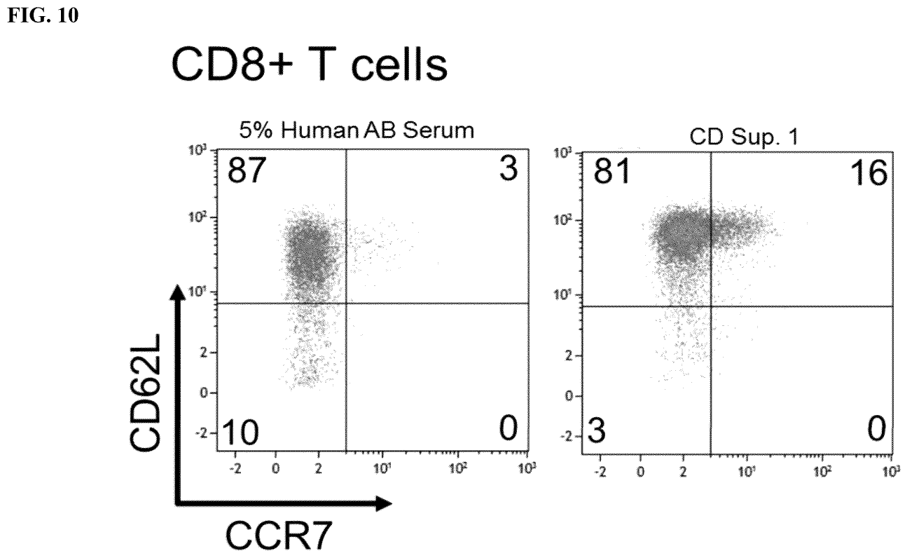

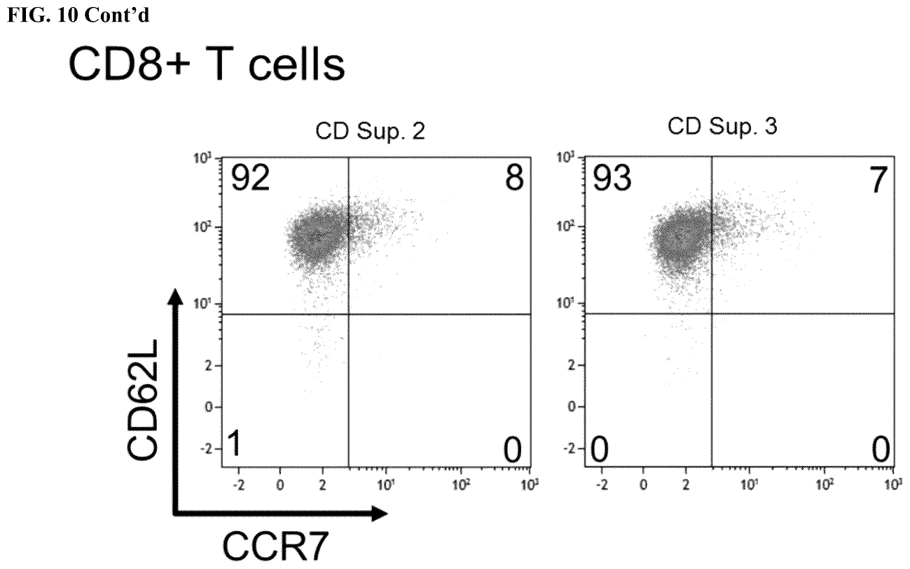

Phenotype: FIG. 7 depicts the gating strategy for differentiation phenotyping. FIG. 8 and Table 10 depict the average changes in CD4+/CD8+ ratios compared to the original subset distribution prior to expansion (Day 0). FIGS. 9 and 10 depict the differentiation status of CD4+ T cells and CD8+ T cells (respectively) expanded in 5% human AB serum and CD Supplements 1, 2, and 3. Results represent that CD4+ and CD8+ T cells cultured with 5% human AB serum lose the CCR7+/CD62L+ phenotype and accumulate the CCR7−/CD62L− phenotype, indicating cellular stress and nutritional deficiencies. Alternatively, CD4+ and CD8+ T cells cultured with CD Supplements 1, 2, and 3 avoid CCR7−/CD62L-accumulation.

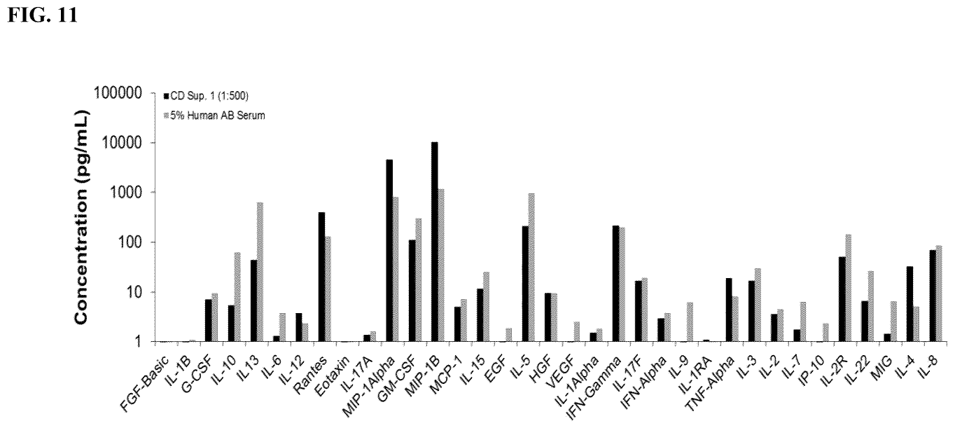

Cytokine Profiles: T cells expanded with DYNABEADS® Human T-Expander CD3/CD28 typically show a predominant Th1-like effector function. IFN-γ is a key mediator of the Th1 immune response. As depicted in FIG. 11, results demonstrate that the cytokine profile of T cells cultured in serum-free medium containing CD Supplement 1 is comparable, if not slightly better, than the profile of T cells cultured in medium containing 5% human AB serum. This is represented by the increase in MIP-1Alpha, decrease in IL-13, IL-10, and IL-6, and no change in IFN-γ and IL-2 production (FIG. 11 and Table 11).

TABLE 1
CD Sup. 1

| | | | Molar Ratios | TC/TFA Molar |
|---|---|---|---|---|
| Component | mol/L | g/L | of CD or FA | Ratio |
| Synthetic Cholesterol | 0.00647 | 2.5 | 0.087 | 0.926:0.074 |
| Methyl-B-Cyclodextrin | 0.06760 | 90 | 0.913 | (1:0.080) |
| Arachidonic Acid | 0.00072 | 0.218 | 0.121 | |
| Linoleic Acid | 0.00508 | 1.425 | 0.852 | |
| Linolenic Acid | 0.00014 | 0.04 | 0.024 | |
| Hot Water | 27.75422 | 0.5 L | N/A | |
| Cold Water | 27.75422 | 0.5 L | N/A | |

TC = Total Cylodextrin, TFA = Total Fatty Acid

TABLE 2
CD Sup. 2

| | | | Molar Ratios | TC/TFA Molar |
|---|---|---|---|---|
| Component | mol/L | g/L | of CD or FA | Ratio |
| Synthetic Cholesterol | 0.00647 | 2.5 | 0.087 | 0.965:0.035 |
| Methyl-ß-Cyclodextrin | 0.0676 | 90 | 0.913 | (1:0.36) |
| Arachidonic Acid | 0.0000657 | 0.02 | 0.0243 | |
| Linoleic Acid | 0.00036 | 0.1 | 0.1331 | |
| Linolenic Acid | 0.00036 | 0.1 | 0.1331 | |
| Myristic Acid | 0.00044 | 0.1 | 0.1626 | |
| Oleic Acid | 0.00035 | 0.1 | 0.1294 | |
| Palmitic Acid | 0.00039 | 0.1 | 0.1441 | |
| Palmitoleic Acid | 0.00039 | 0.1 | 0.1441 | |
| Stearic Acid | 0.00035 | 0.1 | 0.1294 | |
| Hot Water | 27.75422 | 0.5 L | N/A | |
| Cold Water | 27.75422 | 0.5 L | N/A | |

TABLE 3
CD Sup. 3

| | | | Molar Ratios | TC/TFA Molar |
|---|---|---|---|---|
| Component | mol/L | g/L | of CD or FA | Ratio |
| Synthetic Cholesterol | 0.00647 | 2.5 | 0.087 | 0.890:0.110 |
| Methyl-ß-Cyclodextrin | 0.0676 | 90 | 0.913 | (1:0.124) |
| Arachidonic Acid | 0.00032 | 0.099 | 0.0345 | |
| Linoleic Acid | 0.00105 | 0.295 | 0.1147 | |
| Linolenic Acid | 0.00105 | 0.321 | 0.11477 | |
| Myristic Acid | 0.00011 | 0.025 | 0.012027 | |
| Oleic Acid | 0.00273 | 0.771 | 0.298367 | |
| Palmitic Acid | 0.0017 | 0.435 | 0.185797 | |
| Palmitoleic Acid | 0.00017 | 0.042 | 0.01858 | |
| Stearic Acid | 0.00202 | 0.576 | 0.2208 | |
| Hot Water | 27.75422 | 0.5 L | N/A | |
| Cold Water | 27.75422 | 0.5L | N/A | |

TABLE 4
Cultured T Cell Population Doublings with Different Lipid Additions

| | Lipid | CD | CD | CD | CD | CD | CD |
|---|---|---|---|---|---|---|---|
| | Concentrate | Sup. 1 | Sup. 1 | Sup. 1 | Sup. 1 | Sup. 2 | Sup. 2 |
| Days | (1:100) | (1:250) | (1:500) | (1:1000) | (1:1250) | (1:250) | (1:500) |
| 0 | 0 | 0 | 0 | 0 | 0 | 0 | 0 |
| 5 | 1.46 | −3.71 | 1.28 | 1.01 | 1.26 | −4.17 | 0.96 |
| 7 | 3.03 | — | 3.09 | 2.23 | 2.33 | — | 2.75 |
| 10 | 4.74 | — | 5.92 | 5.03 | 4.96 | — | 5.65 |
| 12 | 5.7 | — | 6.79 | 5.88 | 5.067 | — | 6.56 |

| | CD | CD | CD | CD | CD | CD | |
|---|---|---|---|---|---|---|---|
| | Sup. 2 | Sup. 2 | Sup. 3 | Sup. 3 | Sup. 3 | Sup. 3 | No |
| Days | (1:1000) | (1:1250) | (1:250) | (1:500) | (1:1000) | (1:1250) | Lipids |
| 0 | 0 | 0 | 0 | 0 | 0 | 0 | 0 |
| 5 | 0.96 | 0.96 | −3.152 | 1.03 | 0.99 | 1.02 | 0.57 |
| 7 | 2.26 | 2.13 | — | 3.08 | 2.78 | 2.12 | −0.80 |
| 10 | 5.09 | 4.75 | — | 5.79 | 5.53 | 4.56 | −2.08 |
| 12 | 5.64 | 5.22 | — | 6.69 | 6.21 | 5.35 | −2.08 |

TABLE 5
Cultured T Cell Population Doublings-Selected Conditions

| | Lipid | CD | CD | CD | |
|---|---|---|---|---|---|
| | Concentrate | Sup. 1 | Sup. 2 | Sup. 3 | No |
| Days | (1:100) | (1:500) | (1:500) | (1:500) | Lipids |
| 0 | 0 | 0 | 0 | 0 | 0 |
| 5 | 1.46 | 1.28 | 0.96 | 1.03 | 0.57 |
| 7 | 3.03 | 3.09 | 2.75 | 3.08 | −0.80 |
| 10 | 4.74 | 5.92 | 5.65 | 5.79 | −2.08 |
| 12 | 5.7 | 6.79 | 6.54 | 6.69 | −2.08 |

TABLE 6
Total Cultured T Cells (in Millions of Cells) with Different Lipid Additions

| | Lipid | CD | CD | CD | CD | CD | CD |
|---|---|---|---|---|---|---|---|
| | Concentrate | Sup. 1 | Sup. 1 | Sup. 1 | Sup. 1 | Sup. 2 | Sup. 2 |
| Days | (1:100) | (1:250) | (1:500) | (1:1000) | (1:1250) | (1:250) | (1:500) |
| 0 | 1 | 1 | 1 | 1 | 1 | 1 | 1 |
| 5 | 2.76 | 0.08 | 2.43 | 2.01 | 2.40 | 0.06 | 1.95 |
| 7 | 8.17 | — | 8.51 | 4.68 | 5.03 | — | 6.75 |
| 10 | 26.78 | — | 60.63 | 32.70 | 31.04 | — | 50.18 |
| 12 | 51.94 | — | 110.96 | 58.87 | 33.53 | — | 92.83 |

| | CD | CD | CD | CD | CD | CD | |
|---|---|---|---|---|---|---|---|
| | Sup. 2 | Sup. 2 | Sup. 3 | Sup. 3 | Sup. 3 | Sup. 3 | No |
| Days | (1:1000) | (1:1250) | (1:250) | (1:500) | (1:1000) | (1:1250) | Lipids |
| 0 | 1 | 1 | 1 | 1 | 1 | 1 | 1 |
| 5 | 1.95 | 1.95 | 0.12 | 2.04 | 1.98 | 2.03 | 1.16 |
| 7 | 4.80 | 4.38 | — | 8.48 | 6.88 | 4.35 | 6.00 |
| 10 | 34.04 | 26.98 | — | 55.49 | 46.20 | 23.64 | 0.24 |
| 12 | 49.70 | 37.23 | — | 103.20 | 73.92 | 40.90 | 0 |

TABLE 7
Total Cultured T Cells (in Millions of Cells)-Selected Conditions

| | Lipid | CD | CD | CD | |
|---|---|---|---|---|---|
| | Concentrate | Sup. 1 | Sup. 2 | Sup. 3 | No |
| Days | (1:100) | (1:500) | (1:500) | (1:500) | Lipids |
| 0 | 1 | 1 | 1 | 1 | 1 |
| 5 | 2.76 | 2.43 | 1.95 | 2.04 | 1.16 |
| 7 | 8.17 | 8.51 | 6.75 | 8.48 | 6.00 |
| 10 | 26.78 | 60.63 | 50.18 | 55.49 | 0.24 |
| 12 | 51.94 | 110.96 | 92.83 | 103.20 | 0 |

TABLE 8
Cultured T Cell Viability (%) with Different Lipid Additions

| | Lipid | CD | CD | CD | CD | CD | CD |
|---|---|---|---|---|---|---|---|
| | Concentrate | Sup. 1 | Sup. 1 | Sup. 1 | Sup. 1 | Sup. 2 | Sup. 2 |
| Days | (1:100) | (1:250) | (1:500) | (1:1000) | (1:1250) | (1:250) | (1:500) |
| 0 | 96 | 96 | 96 | 96 | 96 | 96 | 96 |
| 5 | 88 | 27 | 85 | 47 | 57 | 28 | 80 |
| 7 | 87 | — | 86 | 72 | 76 | — | 82 |
| 10 | 83 | — | 96 | 93 | 90 | — | 95 |
| 12 | 83 | — | 91 | 82 | 79 | — | 90 |

| | CD | CD | CD | CD | CD | CD | |
|---|---|---|---|---|---|---|---|
| | Sup. 2 | Sup. 2 | Sup. 3 | Sup. 3 | Sup. 3 | Sup. 3 | No |
| Days | (1:1000) | (1:1250) | (1:250) | (1:500) | (1:1000) | (1:1250) | Lipids |
| 0 | 96 | 96 | 96 | 96 | 96 | 96 | 96 |
| 5 | 50 | 55 | 42 | 78 | 54 | 48 | 63 |
| 7 | 72 | 71 | — | 82 | 75 | 67 | 22 |
| 10 | 94 | 92 | — | 96 | 94 | 89 | 11 |
| 12 | 79 | 80 | — | 91 | 90 | 89 | 11 |

TABLE 9
Cultured T Cell Viability (%)-Selected Conditions

| | Lipid | CD | CD | CD | |
|---|---|---|---|---|---|
| | Concentrate | Sup. 1 | Sup. 2 | Sup. 3 | No |
| Days | (1:100) | (1:500) | (1:500) | (1:500) | Lipids |
| 0 | 96 | 96 | 96 | 96 | 96 |
| 5 | 88 | 85 | 80 | 78 | 63 |
| 7 | 87 | 86 | 82 | 82 | 22 |
| 10 | 83 | 96 | 95 | 96 | 11 |
| 12 | 83 | 91 | 90 | 91 | 11 |

TABLE 10
Changes in CD4+/CD8+ Ratios After 10 Days of Culture

| | | | % CD4+ | % CD8+ |
|---|---|---|---|---|
| Conditions | % CD4+ | % CD8+ | (Avg) | (Avg) |
| Day 0 | 52.41 | 47.26 | 52.70 | 46.72 |
| | 50.81 | 46.08 | (SD 2.05) | (SD 0.60) |
| | 54.88 | 46.83 | | |
| 5% Human AB | 68.39 | 26.90 | 67.26 | 28.11 |
| Serum | 67.16 | 28.38 | (SD 1.08) | (SD 1.10) |
| (Day 10) | 66.23 | 29.04 | | |
| CD Sup. 1 | 47.10 | 45.57 | 47.64 | 44.57 |
| (Day 10) | 45.75 | 46.53 | (SD 2.20) | (SD 2.60) |
| | 50.06 | 41.62 | | |
| CD Sup. 2 | 65.44 | 26.27 | 67.07 | 24.91 |
| (Day 10) | 68.27 | 24.32 | (1.46) | (1.18) |
| | 67.50 | 24.13 | | |
| CD Sup. 3 | 62.59 | 32.95 | 62.99 | 30.28 |
| (Day 10) | 63.98 | 28.41 | (0.86) | (2.37) |
| | 62.40 | 29.48 | | |

Average CD4+ and CD8+ ratios derived from three replicates and standard deviations are shown

TABLE 11
T Cell Functionality-Selected Cytokines (Concentration [pg/mL])

| Condition | MIP-1Alpha | IL-6 | IL-10 | IL-13 | IFN-γ | IL-2 |
|---|---|---|---|---|---|---|
| CD Sup. 1 | 4546.93 | 1.30 | 5.44 | 44.42 | 214.87 | 3.60 |
| 5% Human AB | 824.71 | 3.82 | 62.42 | 638.66 | 199.48 | 4.60 |
| Serum | | | | | | |

### Example 2: Culturing T Cells in Serum-Free Medium Containing Lipids

Lipids appear to be the oxidative phosphorylation (OXPHOS) source of energy for T cells grown in serum-free medium and are required for optimal T cell expansion. Primary human T cells from three normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell. Cells were cultured in serum-free medium free of cholesterol and free fatty acids supplemented with one of four lipid supplements or no lipid supplementation as a control. T cells were counted on days 5, 7, 10, and 12 on a Beckman-Coulter Vi-CELL XR analyzer and fed to a density of 5×10⁵ vc/mL on days 3, 5, and 7 and 1×10⁶ vc/mL on day 10. T cell expansion was expressed as either cumulative population doublings or cumulative viable T cells over time. Results in FIGS. 1-4 demonstrate that serum-free medium requires lipid supplementation at a defined concentration to yield optimal T cell expansion. CD Supplements 1, 2, and 3 (1:500) provide optimal T cell expansion compared to Lipid Concentrate (1:100), CD Supplements 1, 2, and 3 at other concentrations, and no lipid supplementation. Results are representative of at least three independent experiments.

### Example 3: Preferential Expansion of the CD8+ T Cell Subset by Serum-Free Medium Containing CD SuP.1

The preferential expansion of CD8+ T cell subsets in serum-free medium containing CD Sup. 1 was analyzed. Primary human T cells from three normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell. Cells were cultured in serum-free medium free of cholesterol and free fatty acids supplemented with one of four lipid supplements or 5% human AB serum as a control. T cells were counted on days 5, 7, and 10 on a Beckman-Coulter Vi-CELL XR analyzer and fed to a density of 5×10⁵ vc/mL on days 3, 5, and 7. T cell expansion was expressed as either cumulative population doublings or cumulative viable T cells over time (FIGS. 1-6). 2×10⁶ cells from each condition were stained with antibodies against CD3, CD4, CD8, CCR7, and CD62L. Sequential gating was used to characterize T cells as central memory (TCM: CCR7+/CD62L+), intermediate (CCR7−/CD62L+), and effector memory (TEM: CCR7−/CD62L−) (FIG. 7).

Flow cytometric analysis was performed on a Beckman-Coulter Gallios analyzer. FIG. 8 results demonstrate the preferential expansion of CD8+ T cells in serum-free medium containing CD Sup. 1 when compared to frequencies in medium containing CD Supplements 2, 3, and 5% human AB serum. Results are representative of at least three independent experiments.

### Example 4: Phenotype of Expanded T Cells in Serum-Free Medium Containing CD Supplements 1, 2, and 3

Primary human T cells from three normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell. Cells were cultured in serum-free medium free of cholesterol and free fatty acids supplemented with one of four lipid supplements or 5% human AB serum as a control. T cells were counted on days 5, 7, and 10 on a Beckman-Coulter Vi-CELL XR analyzer and fed to a density of 5×10⁵ vc/mL on days 3, 5, and 7. T cell expansion was expressed as either cumulative population doublings or cumulative viable T cells over time (FIGS. 1-6). 2×10⁶ cells from each condition were stained with antibodies against CD3, CD4, CD8, CCR7, and CD62L. Sequential gating was used to characterize T cells as central memory (TCM: CCR7+/CD62L+), intermediate (CCR7−/CD62L+), and effector memory (TEM: CCR7−/CD62L−) (FIG. 7). Flow cytometric analysis was performed on a Beckman-Coulter Gallios analyzer. FIG. 9 depicts the differentiation status of CD4+ T cells expanded in serum-free medium containing CD Supplements 1, 2, and 3 vs. 5% human AB serum. FIG. 10 depicts the differentiation status of CD8+ T cells expanded in serum-free medium containing CD Supplements 1, 2, and 3 vs. 5% human AB serum. Results demonstrate a more favorable phenotype of T cells expanded in serum-free medium containing CD Supplements 1, 2, and 3 as defined by greater frequencies of TCM and intermediate subsets at harvest versus control medium. Results are representative of at least three independent experiments.

### Example 5: Cytokine Profiles in Serum-Free Medium Containing CD Sup. 1 vs. Serum-Containing Medium

T cells expanded with DYNABEADS® Human T-Expander CD3/CD28 typically show a predominant Th1-like effector function. IFN-γ is a key mediator of Th1 immune responses. Th1 cytokine profiles were compared between T cells grown in serum-free medium containing CD Sup. 1 vs. serum-containing medium. Primary human T cells from normal donors were negatively isolated from PBMCs with the DYNABEADS® UNTOUCHED™ Human T Cells kit. T cells (seeding density 1×10⁶ vc/mL) were activated with DYNABEADS® Human T-Expander CD3/CD28 at a ratio of 3 beads per T cell and cultured in serum-free medium free of cholesterol and free fatty acids supplemented with CD Sup. 1 (1:500) or control medium X-VIVO™ 15 (Lonza, Cat. No. BE02-054Q) supplemented with 5% human AB serum. T cells were counted on days 5 and 7 on a Beckman-Coulter Vi-CELL XR analyzer and fed to a density of 5×10⁵ vc/mL on days 3, 5, and 7. DYNABEADS® were removed from the cultures on day 11 and cells were spun to remove conditioned medium and rested overnight in fresh medium. One million T cells were re-stimulated with DYNABEADS® CD3 at a 1:1 bead to cell ratio and incubated for 24 hours. Supernatants were collected and processed for analysis with Invitrogen Cytokine Human Magnetic 35-Plex Panel for Luminex™. As depicted in FIG. 11 and Table 11, results demonstrate that T cells expanded in serum-free medium containing CD Sup. 1 show a similar profile of cytokine production with no impairment of IFN-γ production when compared to control serum-containing medium. Results are representative of two independent experiments.

### Example 6

### Materials Methods:

2-Deoxy-D-Glucose Preparation: 2-Deoxy-D-Glucose (2-DG, 164.16 g/mol) was manufactured by (Acros Organics, cat. no. 111980-250). The 2-DG solution was prepared in sterile filtered water at a stock concentration of 100 mM, then aliquoted in Eppendorf tubes at a final volume of 1 mL in each tube.

Cell Culture: T cell isolation: De-identified, frozen apheresis bags from normal donors were obtained from HemaCare Corp. Van Nuys, CA 91406. T cells were negatively isolated from PBMCs with the DYNABEADS® UNTOUCHED™ Human T Cells kit (Thermo Fisher Scientific, Cat. No. 11344D).

T cell activation and expansion: T cells (seeding density 1×10⁶ vc/mL) were activated with DYNABEADS® Human T-Expander CD3/CD28 (Thermo Fisher Scientific, Cat. No. 11141D) at a ratio of 3 beads per T cell and cultured in serum-free medium, animal origin free medium. T cells were counted on days 5, 7, 10, and 12 on a Vi-CELL XR analyzer (Beckman Coulter, Indianapolis IN) and fed with or without 2-DG (0.25 mM, 0.5 mM, 1 mM, 2 mM, and 4 mM) and 5% human AB serum to a density of 5×10⁵ vc/mL on days 3, 5, and 7 and 1×10⁶ vc/mL on day 10.

For some studies, CD8+ and CD4+ T cells were isolated from PBMCs by negative selection using Untouched Human CD8+ and CD4+ T Cells Kits. Naïve and non-naïve T cells were isolated from enriched T cells by positive selection using CD45RA nanobeads (Miltenyi).

Phenotype: Primary human T cells were expanded for 10 days with and without 2-DG and 5% human AB serum. DYNABEADS® were removed from 2×10⁶ cells by magnetic separation. Surface staining was performed with antibodies against CD3 (Invitrogen, Cat. No. CD0329), CD4 (Molecular Probes, Cat. No. A15858), CD8 (Invitrogen, Cat. No. MHCD0828). Flow cytometric analysis was performed on a Gallios flow cytometer and Kaluza software (Beckman Coulter, Indianapolis IN).

Cytokine Profiles: Primary human T cells from normal donors were negatively isolated from PBMCs with the DYNABEADS® UNTOUCHED™ Human T Cells kit (Thermo Fisher Scientific, Cat. No. 11344D). T cells (seeding density 1×10⁶ vc/mL) were activated with DYNABEADS® Human T-Expander CD3/CD28 (Thermo Fisher Scientific, Cat. No. 11141D) at a ratio of 3 beads per T cell and cultured in serum-free and animal origin free medium or control medium X-VIVO™ 15 (Lonza, Cat. No. BE02-054Q) supplemented with 5% human AB serum. T cells were counted on days 5, 7, 10, and 12 on a Vi-CELL XR analyzer (Beckman Coulter, Indianapolis IN) and fed to a density of 5×10⁵ vc/mL on days 3, 5, and 7 and to a density of 1×10⁶ vc/mL on day 10. DYNABEADS® were magnetically removed from cultures on day 10 and cells were spun to remove conditioned medium and rested overnight in fresh medium. One million T cells were re-stimulated with DYNABEADS® Human T-Expander CD3/CD28 (Thermo Fisher Scientific, Cat. No. 11141D) at a 1:1 bead to cell ratio and incubated for 24 hours. Supernatants were collected and processed for analysis with the Cytokine Human Magnetic 35-Plex Panel for Luminex™ (Thermo Fisher Scientific, Cat. No. LHC6005M). Analysis was performed using a MAGPIX® system (Luminex Corporation, Austin TX).

### Results:

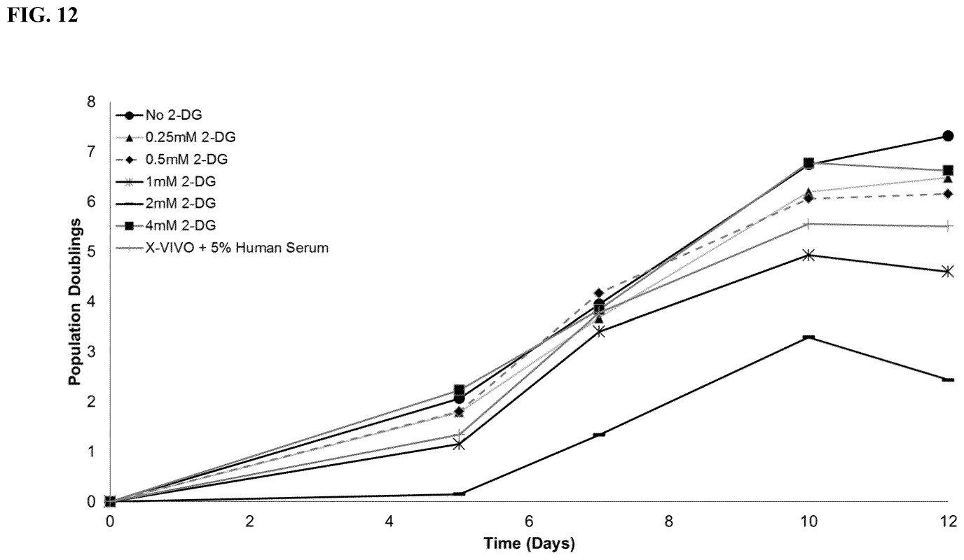

Cell Growth and Viability: T cell expansion is expressed as cumulative population doublings. Results are representative of at least 3 independent experiments. Tables 12, 14-15, 18-19, 22-23 quantify the graphical results displayed in FIGS. 12, 15-16, 19-20, 24-25 respectively. Results in FIG. 12 demonstrate that supplementation with 2-DG does not affect T cell growth. Lower concentrations of 2-DG provide suboptimal T cell expansion (expressed as cumulative population doublings) compared to higher concentrations in serum-free medium. 4 mM 2-DG expanded to about 6 population doublings. In embodiments, the concentration of 2-DG is 4 mM or less. In embodiments, the concentration of 2-DG is 0.5 mM or less. In embodiments, the concentration of 2-DG is 0.25 mM or less.

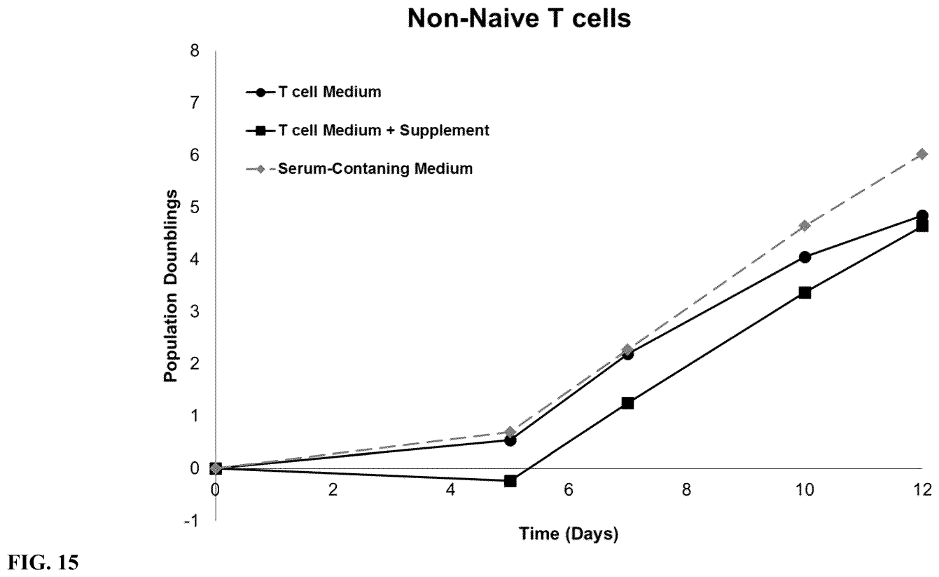

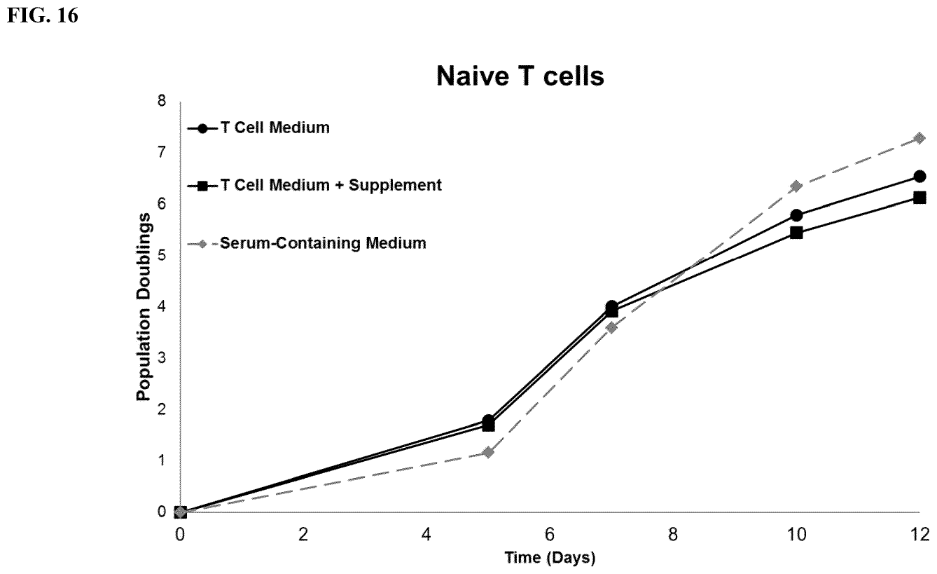

FIGS. 15 and 16 demonstrate the growth curves of naïve and non-naïve T cells. Cell expansion is expressed as cumulative population doublings. Cells were expanded with and without 4 mM 2-DG and in X-VIVO™ 15 with 5% human serum for 12 days. Supplementation with 2-DG did not affect growth of either cell type.

FIGS. 15 and 16 demonstrate the growth curves of naïve and non-naïve T cells. Cell expansion is expressed as cumulative population doublings. Cells were expanded with and without 4 mM 2-DG and in X-VIVO™ 15 with 5% human serum for 12 days. Supplementation with 2-DG did not affect growth of either cell type.

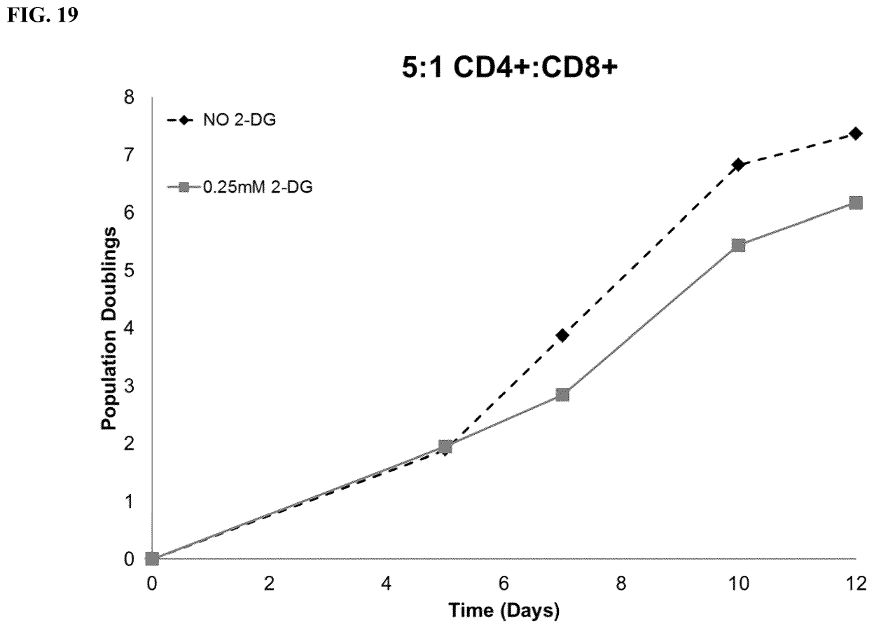

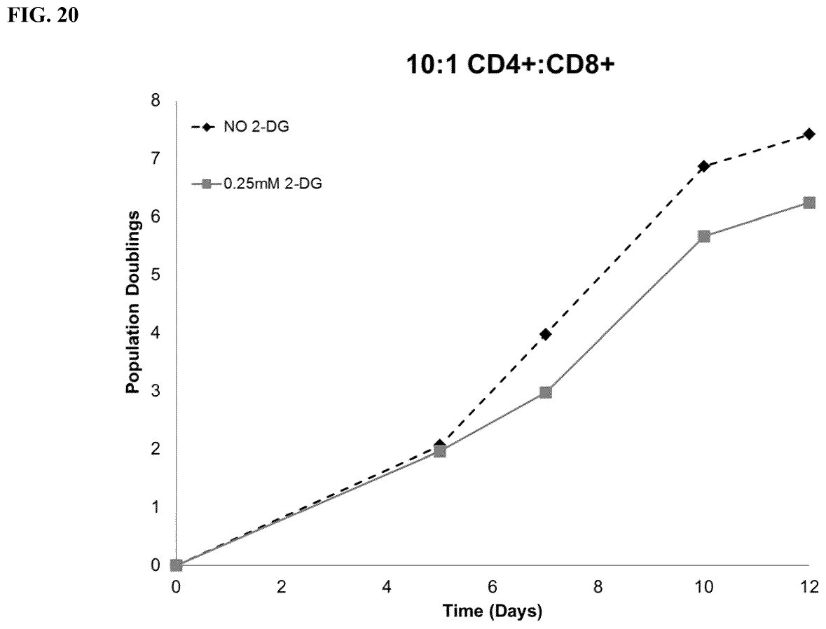

CD4+ and CD8+ T cells were mixed at two different CD4+:CD8+ ratios (5:1 and 10:1) to determine the effect of 2-DG on CD8+ T cells. All conditions with and without 2-DG expanded to about 6 to 7 population doublings. Results illustrate that the effect of 2-DG treatment in cell growth was similar in both mixtures. (FIGS. 19 and 20).

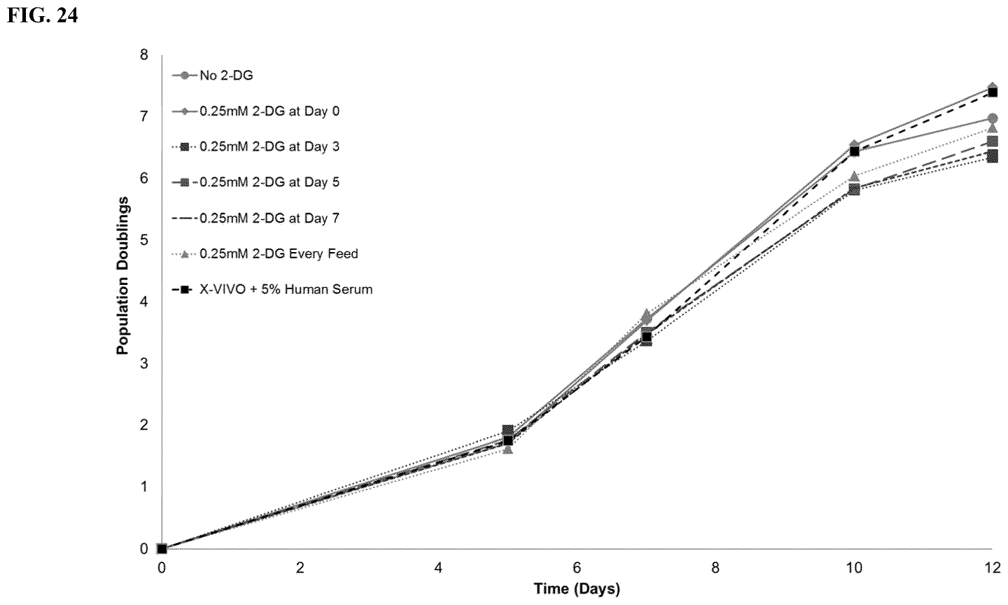

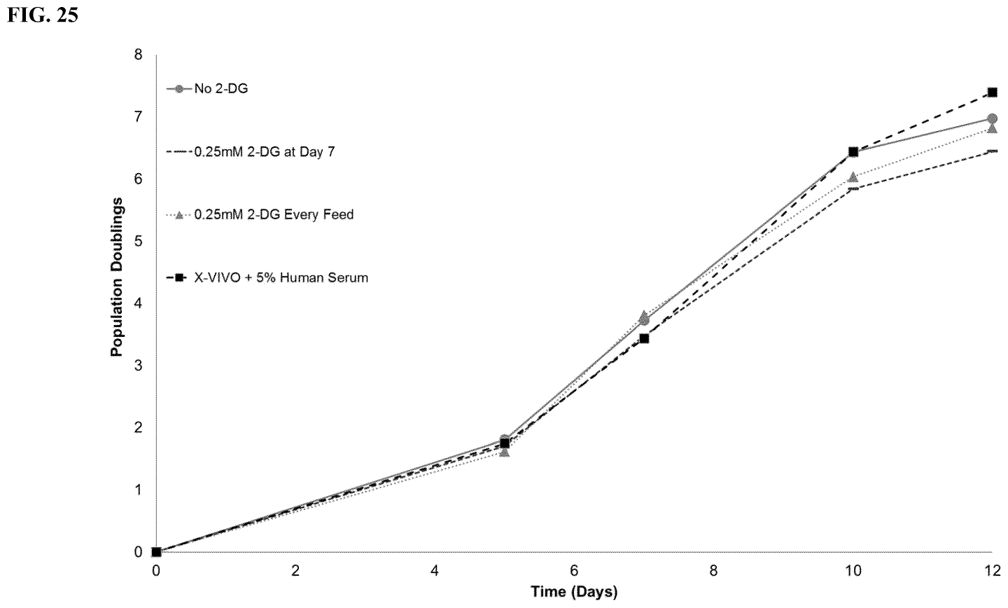

FIG. 25 shows the same growth data as FIG. 24 but with the optimal conditions. T cells were cultured with 0.25 mm 2-DG at different time points (day 0, 3, 5, 7) and every time the cells were fed. All conditions grew to about 6 to 7 population doublings.

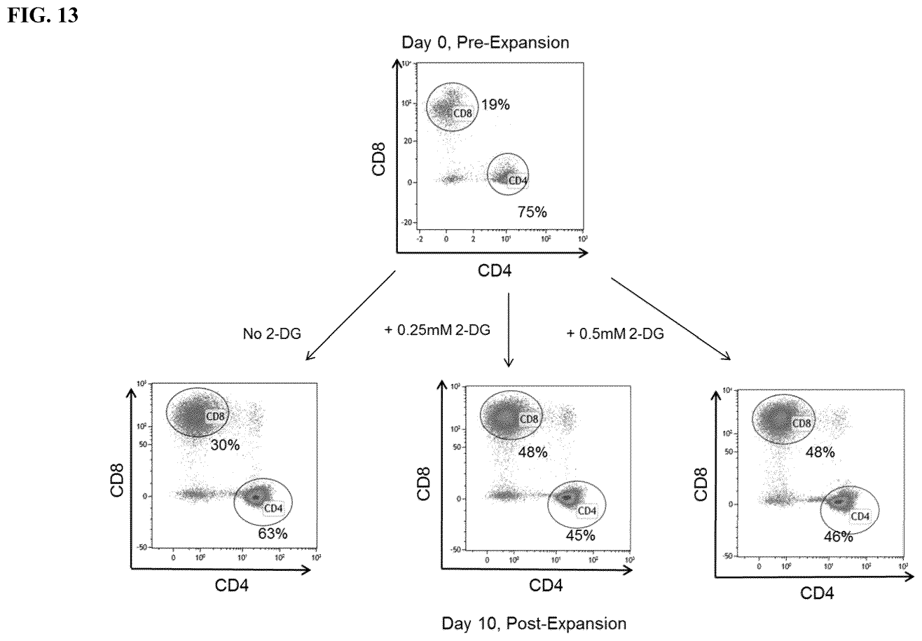

Phenotype: FIG. 13 depicts the gating strategy for differentiation phenotyping. At day 0, pre-expansion, the frequency of CD8+ T cells started at 19%. On day 10, post-expansion, without the addition of 2-DG, the CD8+ T cell population grew to 30%. However, with 0.25 mM and 0.5 mM 2-DG, the CD8+ T cell population increased to 48%.

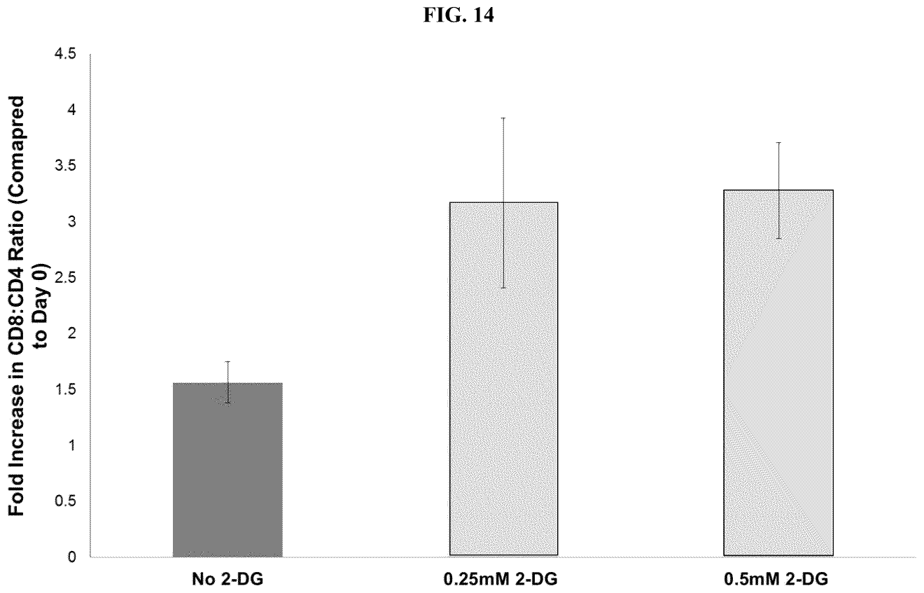

FIG. 14. depicts CD8+:CD4+ ratios compared to the original subset distribution prior to expansion (Day 0). Results represent that there was about 3.2 fold increase in CD8+:CD4+ T cell ratio compared to day 0.

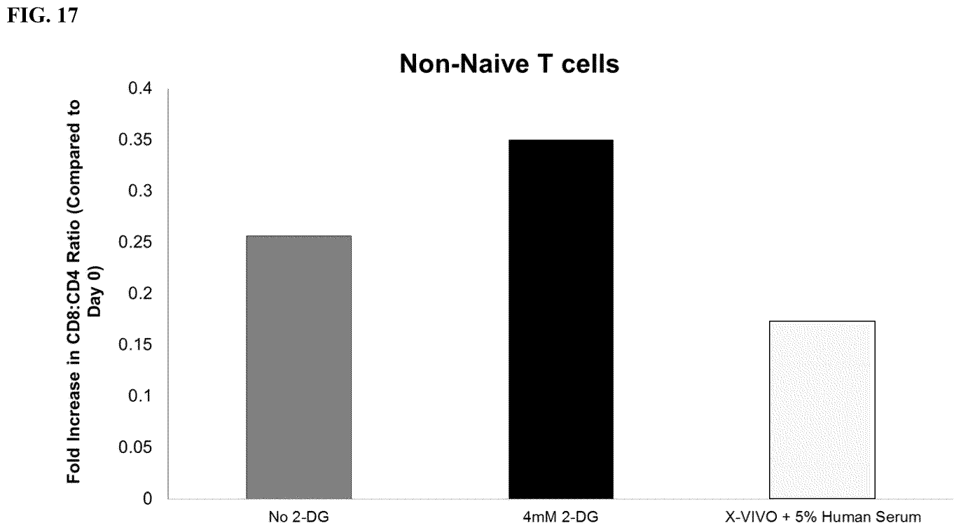

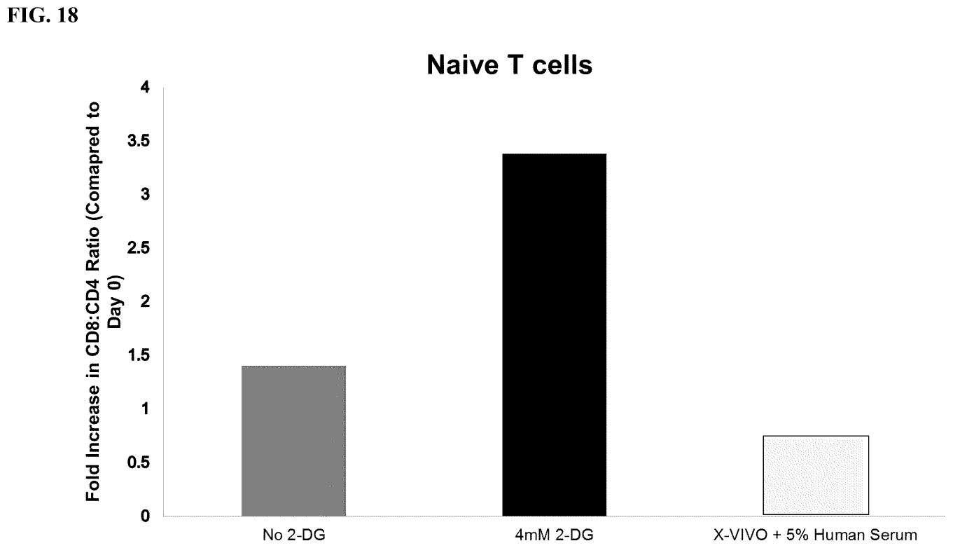

FIGS. 17 and 18 show CD8+:CD4+ ratios compared to the original subset distribution prior to expansion (Day 0). Results demonstrate that there is a 3.4 fold increase in CD8+:CD4+ T cell ratio in naïve T cells where there was a lesser effect in non-naïve T cells.

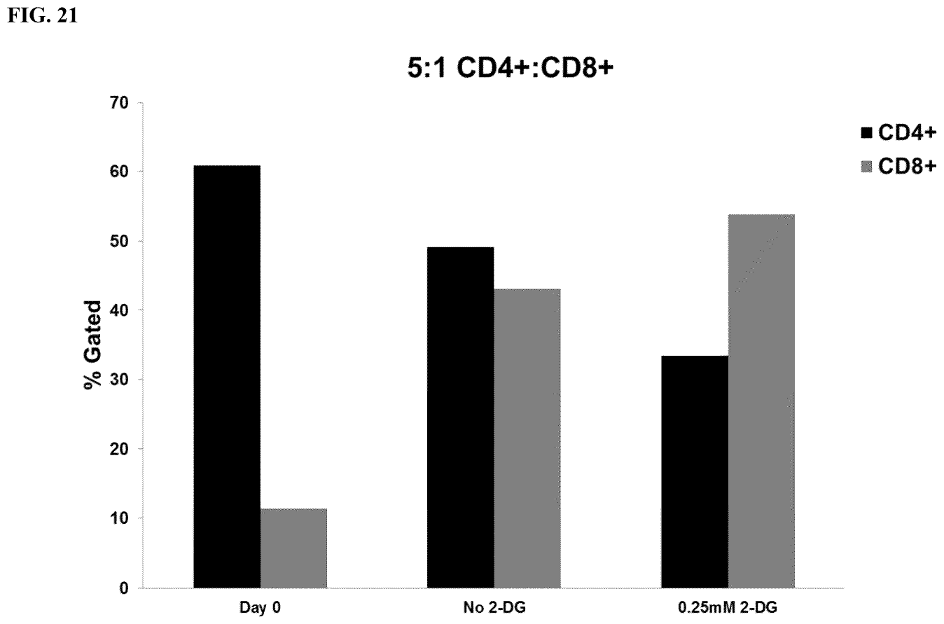

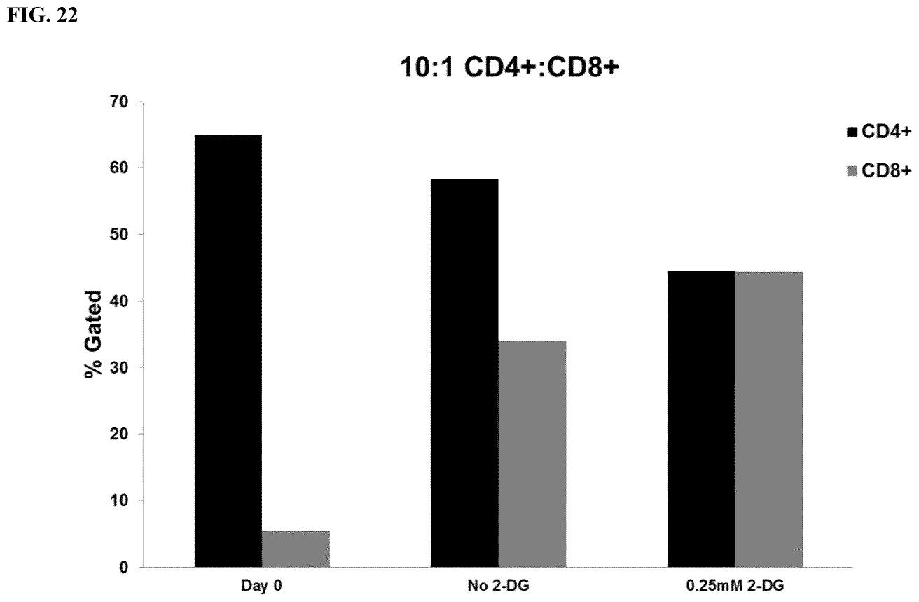

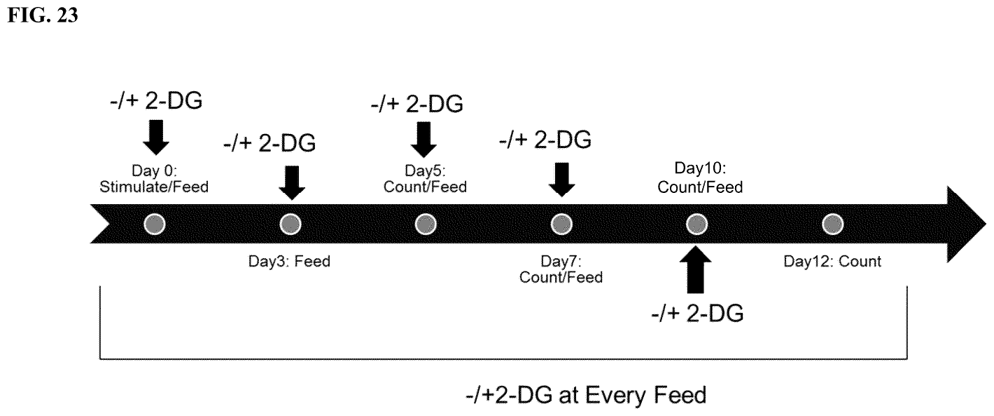

FIGS. 21 and 22 depict the average changes in CD4+/CD8+ ratios compared to the original subset distribution prior to expansion, day 0, which represents frequency of the two populations prior to expansion. 2-DG was able to correct large deficits in CD8+ T cells compared to day 0.

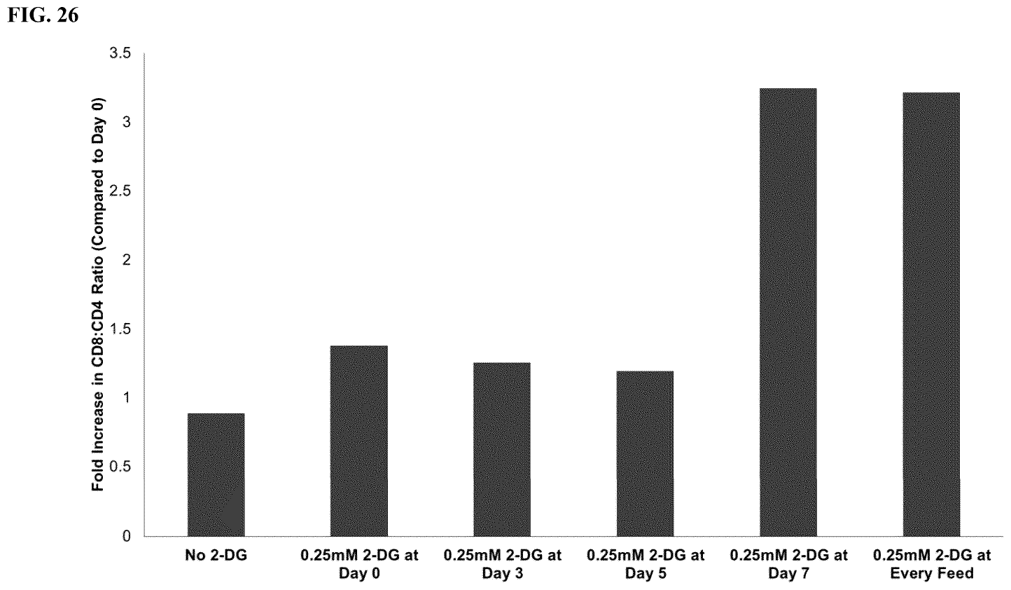

FIG. 26 represents the fold increase in CD8+:CD4+ T cell ratio compared to day 0. Results show that culturing the cells with 0.25 mM 2-DG on day 7 only and at every time the cells were fed resulted in the same 3-fold increase in CD8+ T cells.

### Cytokine Profiles:

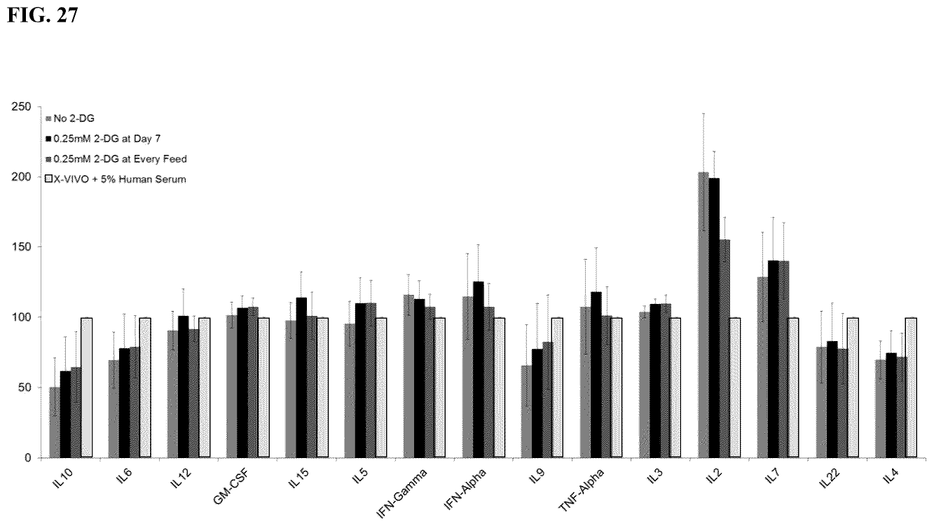

T cells were expanded for 12 days and re-stimulated with DYNABEADS® Human T-Expander CD3/CD28. Cytokine production upon re-stimulation was assessed with Invitrogen Cytokine Human Magnetic 35-Plex Panel for LUMINEX™. Fifteen cytokines shown out of 35-plex assay. All values were normalized relative to X-VIVO™ 15 supplemented with 5% human AB serum. Results demonstrate that there is no change in cytokine production which means that 2-DG does not alter the function of the cells as measured by multiplexed cytokine assay FIG. 27.

TABLE 12
Cultured T Cell Population Doublings with and without 2-DG

| | | | | | | | 5% |
|---|---|---|---|---|---|---|---|
| | | | | | | | Human |
| | No | 0.25 mM | 0.5 mM | 1 mM | 2 mM | 4 mM | AB |
| Days | 2-DG | 2-DG | 2-DG | 2-DG | 2-DG | 2-DG | Serum |
| 0 | 0 | 0 | 0 | 0 | 0 | 0 | 0 |
| 5 | 2.07 | 1.79 | 1.81 | 1.15 | 0.15 | 2.24 | 1.34 |
| 7 | 3.94 | 3.67 | 4.17 | 3.40 | 1.33 | 3.84 | 3.78 |
| 10 | 6.75 | 6.20 | 6.07 | 4.93 | 3.29 | 6.78 | 5.55 |
| 12 | 7.32 | 6.49 | 6.16 | 4.60 | 2.44 | 6.63 | 5.51 |

TABLE 13
Fold Increase in CD8+ to CD4+ Ratios After 10 Days of Culture

| | | | % Ratios | | % Ratios | Fold | Standard |
|---|---|---|---|---|---|---|---|
| | % | % | (CD8+/ | Fold | (CD8+/CD4+) | Increase | Deviation |
| Conditions | CD4+ | CD8+ | CD4+) | Increase | (Avg) | (Avg) | (Avg) |
| Day 0 | 65.69 | 29.65 | 0.451362 | | 0.7 | 1 | |
| | 54.89 | 42.99 | 0.783203 | | | | |
| | 55 | 42.75 | 0.777273 | | | | |
| No 2-DG | 57.84 | 35.84 | 0.61964 | 1.37 | 1.1 | 1.6 | 0.2 |
| (Day 10) | 40.1 | 54.69 | 1.36384 | 1.74 | | | |
| | 41.42 | 50.76 | 1.23 | 1.58 | | | |
| 0.25 mM 2-DG | 39.21 | 53.46 | 1.36 | 3.02 | 2.1 | 3.2 | 0.8 |
| (Day 10) | 32.39 | 63.2 | 1.95 | 2.49 | | | |
| | 22.68 | 70.32 | 3.10 | 3.99 | | | |
| 0.5 mM 2-DG | 37.08 | 55.04 | 1.48 | 3.29 | 2.2 | 3.6 | 0.4 |
| (Day 10) | 29.85 | 66.57 | 2.23 | 2.85 | | | |
| | 23.85 | 68.64 | 2.88 | 3.7 | | | |

TABLE 14
Cultured Non-Naïve T Cell Population
Doublings with and without 2-DG

| | No | 4 mM | 5% Human |
|---|---|---|---|
| Days | 2-DG | 2-DG | AB Serum |
| 0 | 0 | 0 | 0 |
| 5 | 0.54 | −0.24 | 0.69 |
| 7 | 2.19 | 1.25 | 2.27 |
| 10 | 4.05 | 3.37 | 4.64 |
| 12 | 4.84 | 4.64 | 6.02 |

TABLE 15
Cultured Naïve T Cell Population
Doublings with and without 2-DG

| | No | 4 mM | 5% Human |
|---|---|---|---|
| Days | 2-DG | 2-DG | AB Serum |
| 0 | 0 | 0 | 0 |
| 5 | 1.79 | 1.70 | 1.16 |
| 7 | 4.00 | 3.92 | 3.60 |
| 10 | 5.80 | 5.45 | 6.35 |
| 12 | 6.54 | 6.13 | 7.29 |

TABLE 16
Fold Increase in CD8+ to CD4+ Ratios After 10
Days of Culture in Non-Naïve T Cells

| | % | % | % CD4+ | % CD8+ | % Ratios | Fold |
|---|---|---|---|---|---|---|
| Conditions | CD4+ | CD8+ | (Avg) | (Avg) | (CD8+/CD4+) | Increase |
| Day 0 | 64.79 | 27.92 | 64.79 | 27.92 | 0.43 | 1 |
| No 2-DG | 81.28 | 35.84 | 82.52 | 9.11 | 0.11 | 0.25 |
| | 83.75 | 54.69 | | | | |
| 4 mM 2-DG | 80.85 | 12.23 | 81.08 | 12.23 | 0.15 | 0.35 |
| | 81.31 | 12.23 | | | | |
| 5% Human AB | 79.02 | 5.94 | 79.65 | 5.99 | 0.07 | 0.17 |
| Serum | 80.27 | 6.05 | | | | |

TABLE 17
Fold Increase in CD8+ to CD4+ Ratios After 10 Days of Culture in Naïve T Cells

| | % | % | % CD4+ | % CD8+ | % Ratios | Fold |
|---|---|---|---|---|---|---|
| Conditions | CD4+ | CD8+ | (Avg) | (Avg) | (CD8+/CD4+) | Increase |
| Day 0 | 59.69 | 36.66 | 59.69 | 36.66 | 0.61 | 1 |
| No 2-DG | 48.80 | 41.02 | 48.215 | 41.55 | 0.86 | 1.40 |
| | 47.63 | 42.02 | | | | |
| 4 mM 2-DG | 25.23 | 53.26 | 59.69 | 36.66 | 2.07 | 3.38 |
| | 25.82 | 52.71 | | | | |
| 5% Human AB | 61.78 | 25.88 | 61.08 | 27.67 | 0.45 | 0.74 |
| Serum | 60.638 | 29.46 | | | | |

TABLE 18
Cultured T Cell Population
Doublings at 5:1 CD4+:CD8+ Cells

| | No | 0.25 mM |
|---|---|---|
| Days | 2-DG | 2-DG |
| 0 | 0 | 0 |
| 5 | 3.87 | 2.84 |
| 7 | 6.82 | 5.44 |
| 10 | 7.36 | 6.17 |
| 12 | 1.89 | 1.95 |

TABLE 19
Cultured T Cell Population
Doublings at 10:1 CD4+:CD8+ Cells

| | No | 0.25 mM |
|---|---|---|
| Days | 2-DG | 2-DG |
| 0 | 0 | 0 |
| 5 | 2.06 | 1.97 |
| 7 | 3.98 | 2.98 |
| 10 | 6.87 | 5.67 |
| 12 | 7.42 | 6.25 |

TABLE 20
Changes in CD4+/CD8+ Ratios After 10 Days of Culture
(5:1 CD4+ CD8+ Ratio)

| | | | % CD4+ | % CD8+ |
|---|---|---|---|---|
| Conditions | % CD4+ | % CD8+ | (Avg) | (Avg) |
| Day 0 | 60.88 | 11.42 | 60.88 | 11.42 |
| No 2-DG | 49.33 | 43.18 | 49.16 | 43.05 |
| | 48.99 | 42.92 | | |
| 0.25 mM 2-DG | 32.91 | 53.50 | 33.39 | 53.83 |
| | 33.88 | 54.17 | | |

TABLE 21
Changes in CD4+/CD8+ Ratios After 10 Days of Culture
(10:1 CD4+ CD8+ Ratio)

| | % | % | % CD4+ | % CD8+ |
|---|---|---|---|---|
| Conditions | CD4+ | CD8+ | (Avg) | (Avg) |
| Day 0 | 65.03 | 5.42 | 65.03 | 5.42 |
| No 2-DG | 57.88 | 33.92 | 58.195 | 33.97 |
| (Day 10) | 58.51 | 34.02 | | |
| 0.25 mM 2-DG | 44.25 | 45.35 | 44.57 | 44.45 |
| (Day 10) | 44.89 | 43.55 | | |

TABLE 22
Cultured T Cell Population Doublings with Culturing
2-DG at days 0, 3, 5, 7 and at every feed

| | | | | | | 0.25 mM | |
|---|---|---|---|---|---|---|---|
| | | 0.25 mM | 0.25 mM | 0.25 mM | 0.25 mM | 2-DG at | |
| | | 2-DG at | 2-DG at | 2-DG at | 2-DG at | Day at | 5% Human |
| Days | No 2-DG | Day 0 | Day 3 | Day 5 | Day 7 | every feed | AB Serum |
| 0 | 0 | 0 | 0 | 0 | 0 | 0 | 0 |
| 5 | 1.81 | 1.71 | 1.90 | 1.75 | 1.70 | 1.61 | 1.74 |
| 7 | 3.72 | 3.69 | 3.36 | 3.50 | 3.48 | 3.80 | 3.44 |
| 10 | 6.43 | 6.53 | 5.80 | 5.82 | 5.83 | 6.03 | 6.43 |
| 12 | 6.97 | 7.47 | 6.34 | 6.59 | 6.44 | 6.82 | 7.39 |

TABLE 23
Cultured T Cell Population Doublings with Culturing 2-DG
at day 7 and at every feed

| | | 0.25 mM | 0.25 mM | |
|---|---|---|---|---|
| | No | 2-DG | 2-DG at Day | 5% Human |
| Days | 2-DG | at Day 7 | at every feed | AB Serum |
| 0 | 0 | 0 | 0 | 0 |
| 5 | 1.81 | 1.70 | 1.61 | 1.74 |
| 7 | 3.72 | 3.48 | 3.80 | 3.44 |
| 10 | 6.43 | 5.83 | 6.03 | 6.43 |
| 12 | 6.97 | 6.44 | 6.82 | 7.39 |

TABLE 24
Fold Increase in CD8+ to CD4+ Ratios of 2-DG at Different
Time Points and Every Feed After 10 Days of Culture

| | % | % | % Ratios | Fold |
|---|---|---|---|---|
| Conditions | CD4+ | CD8+ | (CD8+/CD4+) | Increase |
| Day 0 | 78.4 | 17.41 | 0.22 | 1 |
| No 2-DG | 82.21 | 16.13 | 0.20 | 0.88 |
| 0.25 mM | 74.78 | 22.88 | 0.31 | 1.38 |
| 2-DG at Day 0 | | | | |
| 0.25 mM | 70.62 | 25.76 | 0.36 | 1.64 |
| 2-DG at Day 3 | | | | |
| 0.25 mM | 76.18 | 21.26 | 0.28 | 1.26 |
| 2-DG at Day 5 | | | | |
| 0.25 mM | 73.28 | 23.91 | 0.33 | 1.47 |
| 2-DG at Day 7 | | | | |
| 0.25 mM | 76.76 | 20.35 | 0.27 | 1.19 |
| 2-DG at Every | | | | |
| Feed | | | | |

### Example 7

### Materials Methods:

2-Deoxy-D-Glucose Preparation: 2-Deoxy-D-Glucose (2-DG, 164.16 g/mol) is manufactured by Acros Organics (part of Thermo Fisher Scientific). The 2-DG solution was prepared in sterile filtered water at a stock concentration of 100 mM, then aliquoted in Eppendorf tubes at a final volume of 1 mL in each tube.

Cell Culture: T cell isolation: De-identified, frozen apheresis bags from normal donors were obtained from HemaCare. T cells were negatively isolated from PBMCs with the DYNABEADS® UNTOUCHED™ Human T Cells kit (Thermo Fisher Scientific, Cat. No. 11344D).

T cell activation and expansion: T cells (seeding density 1×10⁶ vc/mL) were activated with DYNABEADS® Human T-Expander CD3/CD28 (Thermo Fisher Scientific, Cat. No. 11141D) at a ratio of 3 beads per T cell and cultured in serum-free medium, animal origin free medium. T cells were counted on days 5, 7, 10, and 12 on a Vi-CELL XR analyzer (Beckman Coulter, Indianapolis IN) and fed with or without 2-DG (0.25 mM, 0.5 mM, 1 mM, 2 mM, and 4 mM) and 5% human AB serum to a density of 5×10⁵ vc/mL on days 3, 5, and 7 and 1×10⁶ vc/mL on day 10.

For some studies, CD8+ and CD4+ T cells were isolated from PBMCs by negative selection using Untouched Human CD8+ and CD4+ T Cells Kits. Naïve and non-naïve T cells were isolated from enriched T cells by positive selection using CD45RA nanobeads (Miltenyi).

Phenotype: Primary human T cells were expanded for 10 days with and without 2-DG and 5% human AB serum. DYNABEADS® were removed from 2×10⁶ cells by magnetic separation. Surface staining was performed with antibodies against CD3 (Invitrogen, Cat. No. CD0329), CD4 (Molecular Probes, Cat. No. A15858), CD8 (Invitrogen, Cat. No. MHCD0828). Flow cytometric analysis was performed on a Gallios flow cytometer and Kaluza software (Beckman Coulter, Indianapolis IN).

Cytokine Profiles: Primary human T cells from normal donors were negatively isolated from PBMCs with the DYNABEADS® UNTOUCHED™ Human T Cells kit (Thermo Fisher Scientific, Cat. No. 11344D). T cells (seeding density 1×10⁶ vc/mL) were activated with DYNABEADS® Human T-Expander CD3/CD28 (Thermo Fisher Scientific, Cat. No. 11141D) at a ratio of 3 beads per T cell and cultured in serum-free and animal origin free medium or control medium X-VIVO™ 15 (Lonza, Cat. No. BE02-054Q) supplemented with 5% human AB serum. T cells were counted on days 5, 7, 10, and 12 on a Vi-CELL XR analyzer (Beckman Coulter, Indianapolis IN) and fed to a density of 5×10⁵ vc/mL on days 3, 5, and 7 and to a density of 1×10⁶ vc/mL on day 10. DYNABEADS® were magnetically removed from cultures on day 10 and cells were spun to remove conditioned medium and rested overnight in fresh medium. One million T cells were re-stimulated with DYNABEADS® Human T-Expander CD3/CD28 (Thermo Fisher Scientific, Cat. No. 11141D) at a 1:1 bead to cell ratio and incubated for 24 hours. Supernatants were collected and processed for analysis with the Cytokine Human Magnetic 35-Plex Panel for Luminex™ (Thermo Fisher Scientific, Cat. No. LHC6005M). Analysis was performed using a MAGPIX® system (Luminex Corporation, Austin TX).

### Results:

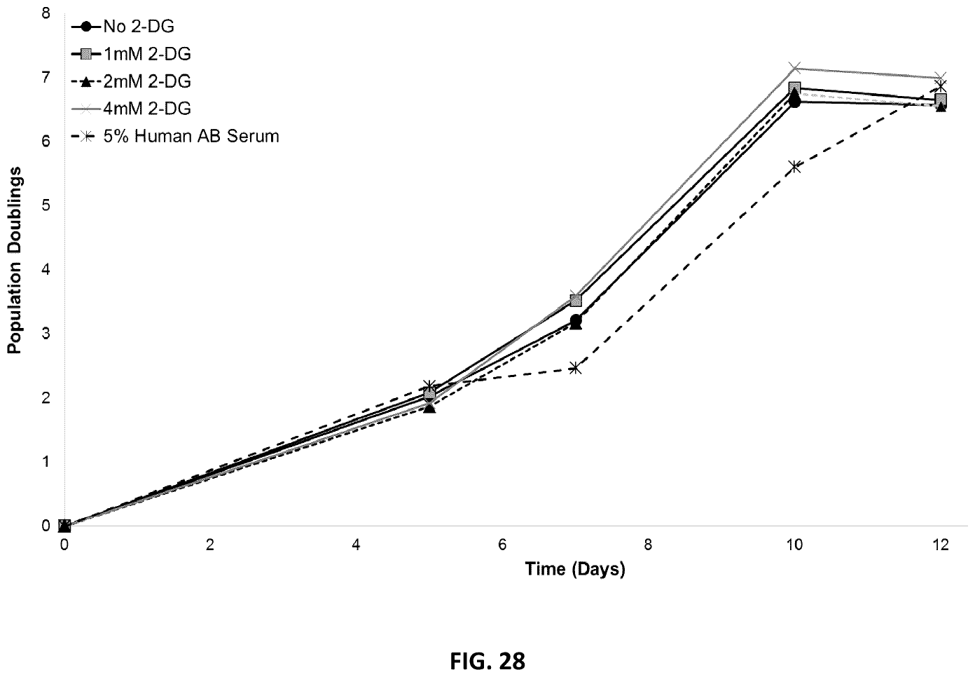

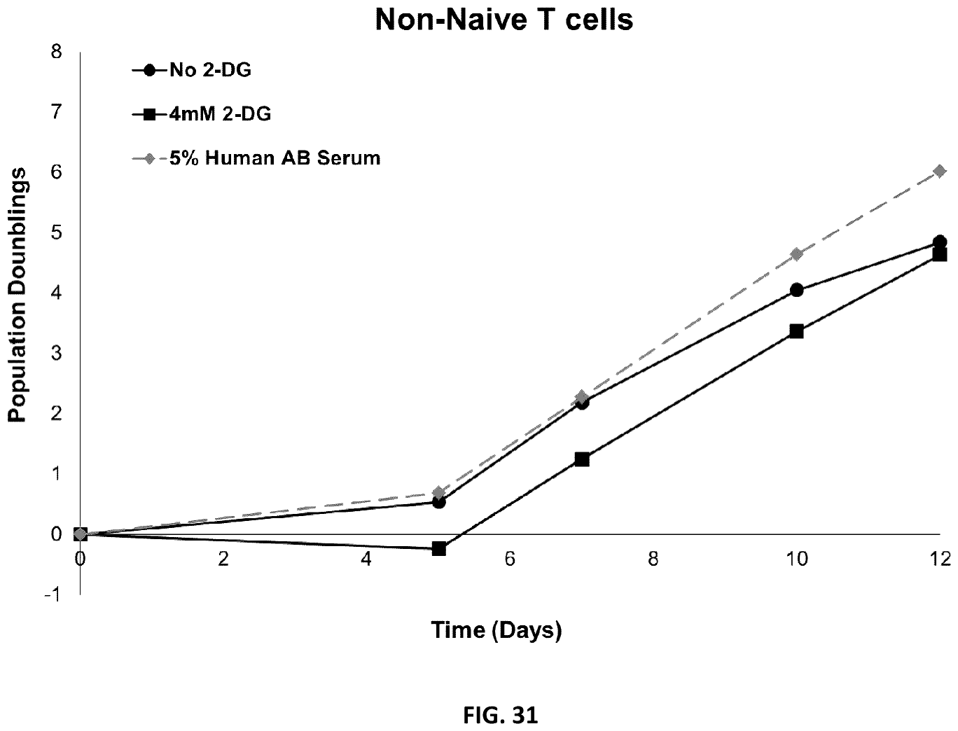

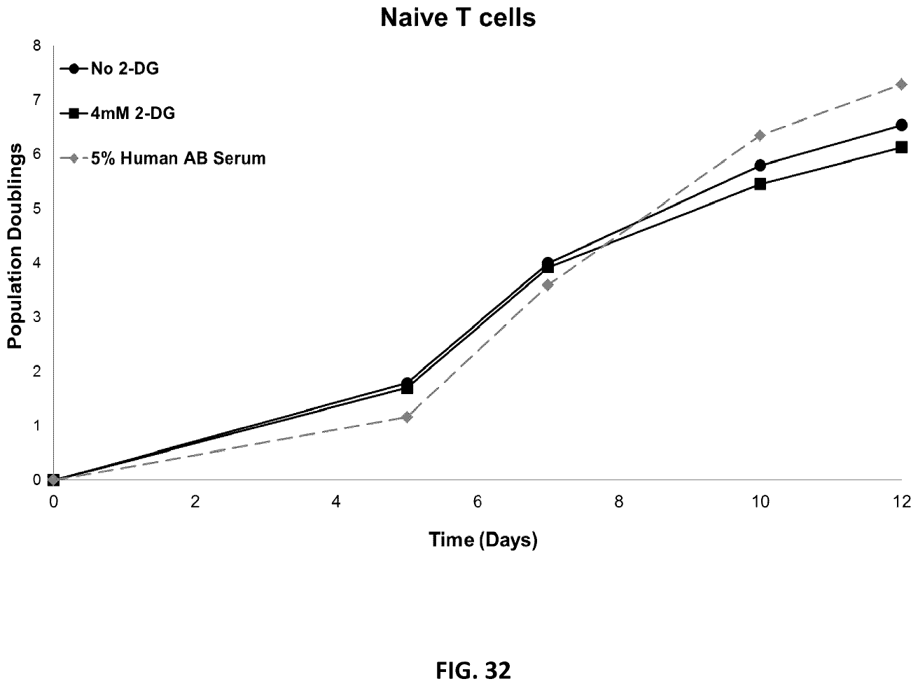

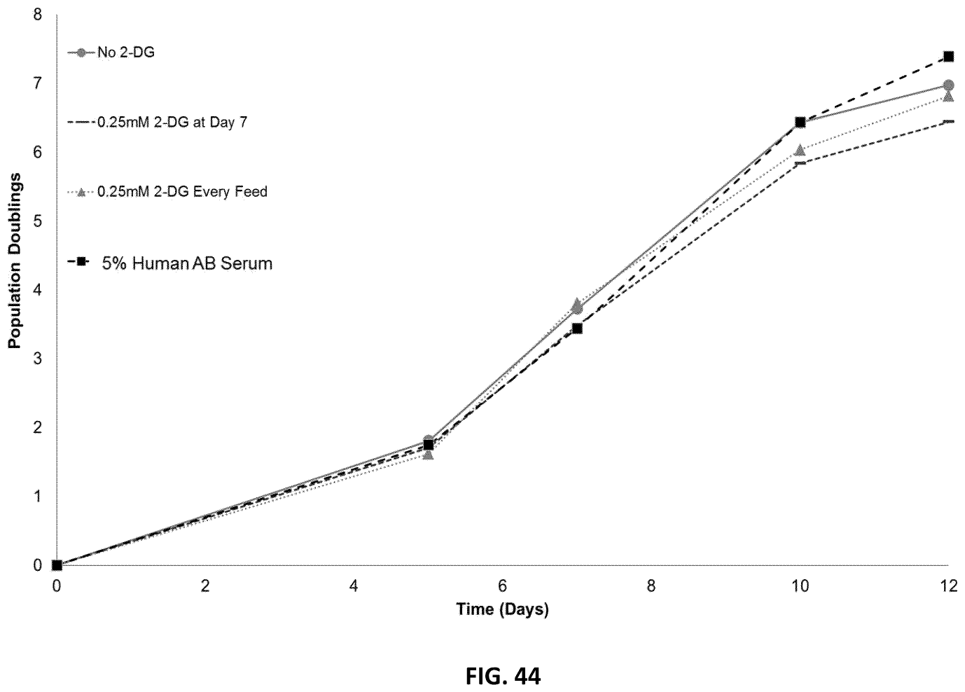

Cell Growth and Viability: T cell expansion is expressed as cumulative population doublings. Results are representative of at least 3 independent experiments. Tables 25, 27-28, 31, 33-34, 37-38, and 40-41 quantify the graphical results displayed in FIGS. 28, 31-32, 35, 38-39, 43-44, and 46-47 respectively. FIG. 28 illustrates a dose-response of 2-DG (0, 1 mM, 2 mM, and 4 mM) in Bulk T cells. Results demonstrate that supplementation with 2-DG does not affect T cell growth. FIGS. 31 and 32 demonstrate the growth curves of naïve and non-naïve T cells. Cell expansion is expressed as cumulative population doublings. Cells were expanded with and without 4 mM 2-DG and with 5% human serum for 12 days. Supplementation with 2-DG did not affect growth in naïve T cells, however, there was a modest effect on the non-naïve T cells.

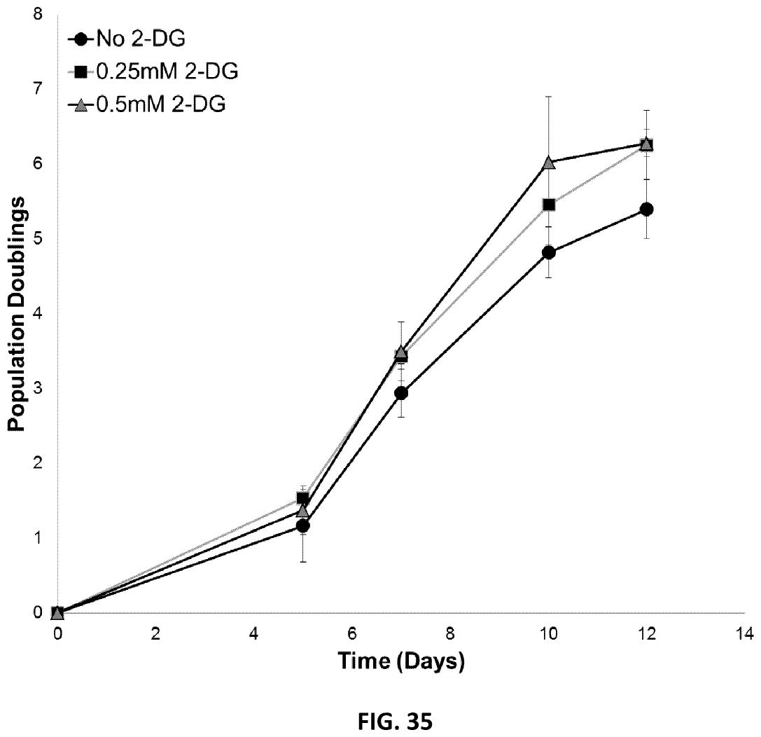

FIG. 35 illustrates a smaller dose-response of 2-DG (0.25 mM and 0.5 mM) in Bulk T cells. We tested a lower concentrations of 2-DG because it seems that different concentrations of 2-DG are required for different applications (The mix of CD4+ T cells with CD8+ T cells at set ratios). Results show that supplementation with 2-DG does not affect T cell growth even with lower concentrations.

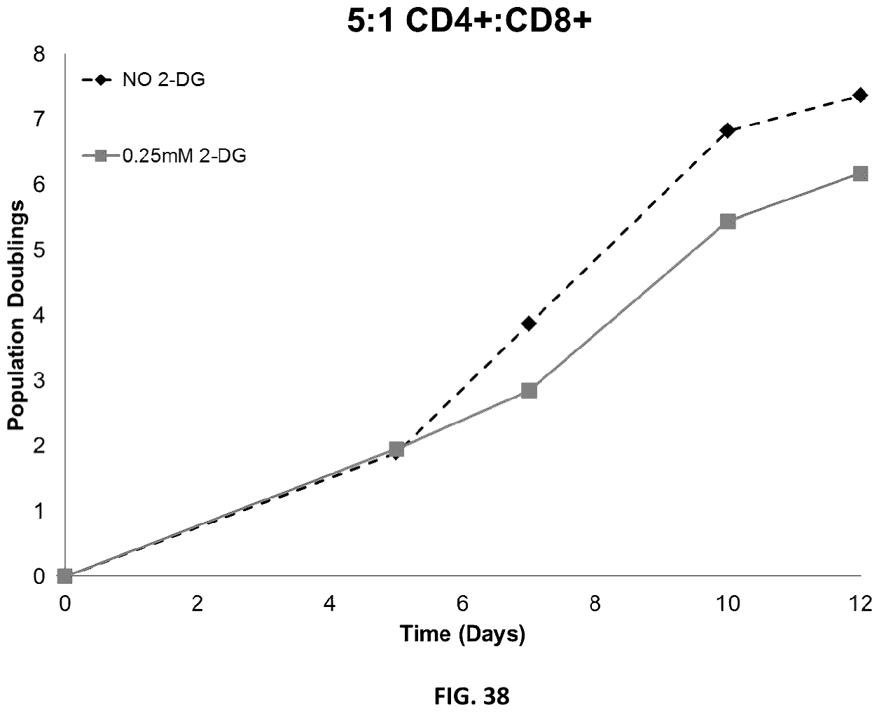

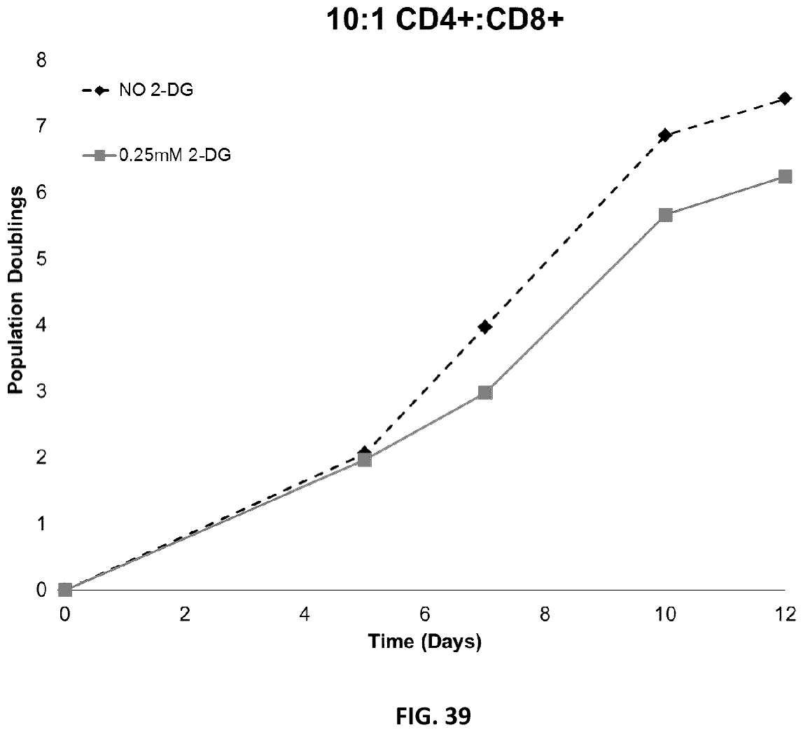

In FIGS. 38 and 39, CD4+ and CD8+ T cells were mixed at two different CD4:CD8 ratios (5:1 and 10:1) to determine the effect of 2-DG on CD8+ T cells. All conditions with and without 2-DG expanded to about 6 to 7 population doublings. Results illustrate that the effect of 2-DG treatment in cell growth was similar in both mixtures.

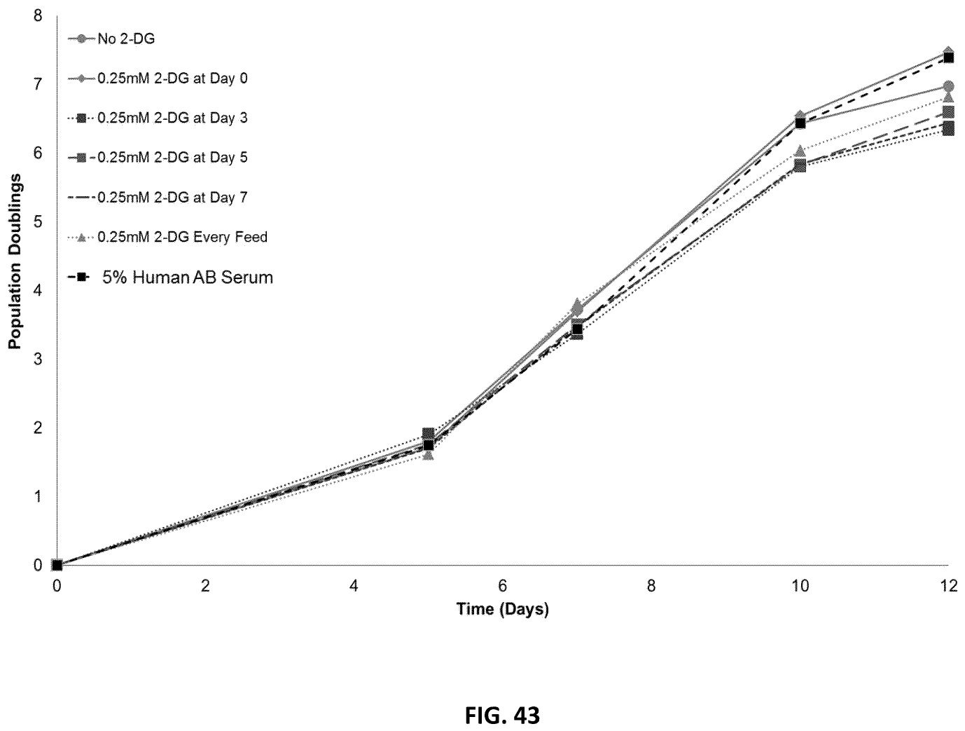

FIG. 43 shows the same growth data as FIG. 17 but with the optimal conditions. T cells were cultured with 0.25 mm 2-DG at different time points (day 0, 3, 5, 7) and every time the cells were fed. All conditions grew to about 6 to 7 population doublings.

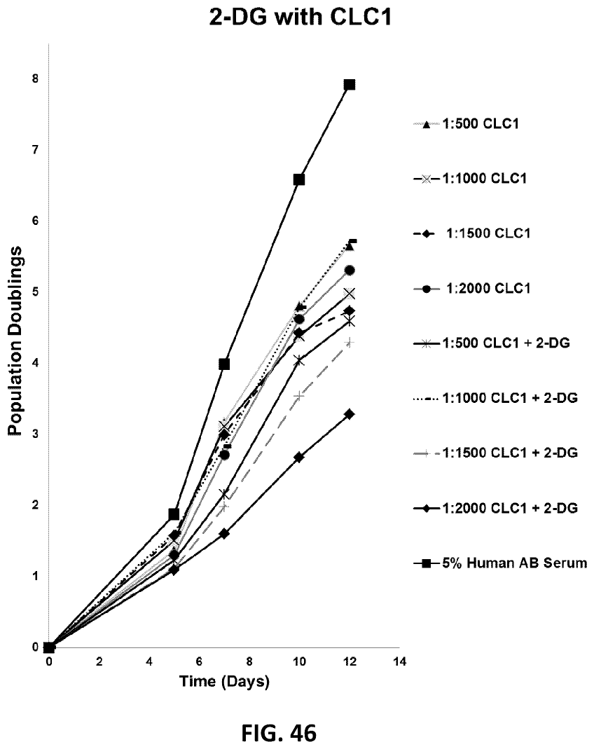

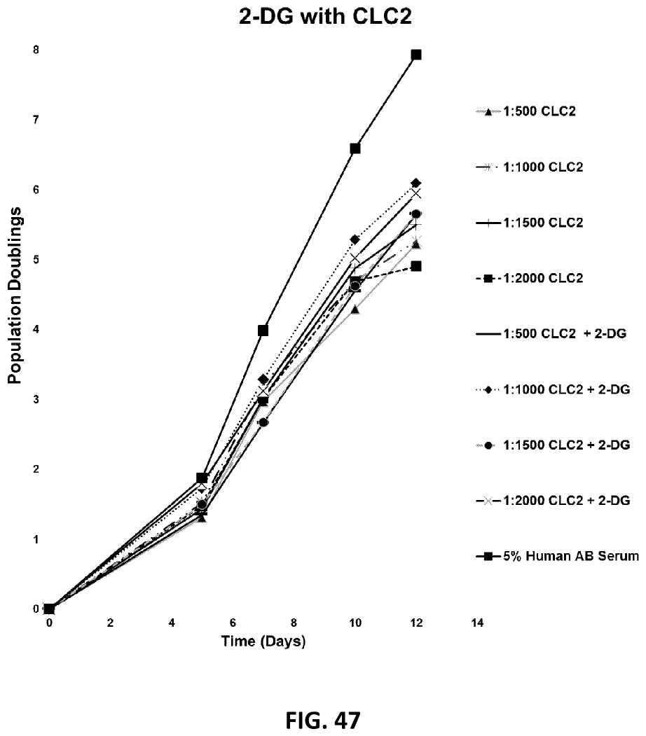

FIGS. 46 and 47 illustrate a dose response of CD Lipid Concentrate 1 (CLC1) and CD Lipid Concentrate 2 (CLC2) with 2-DG respectively. Results show that 1:1000 CLC1 and 1:1000 CLC2 with and without 2-DG demonstrate the optimal growth in T cells.

### Phenotype:

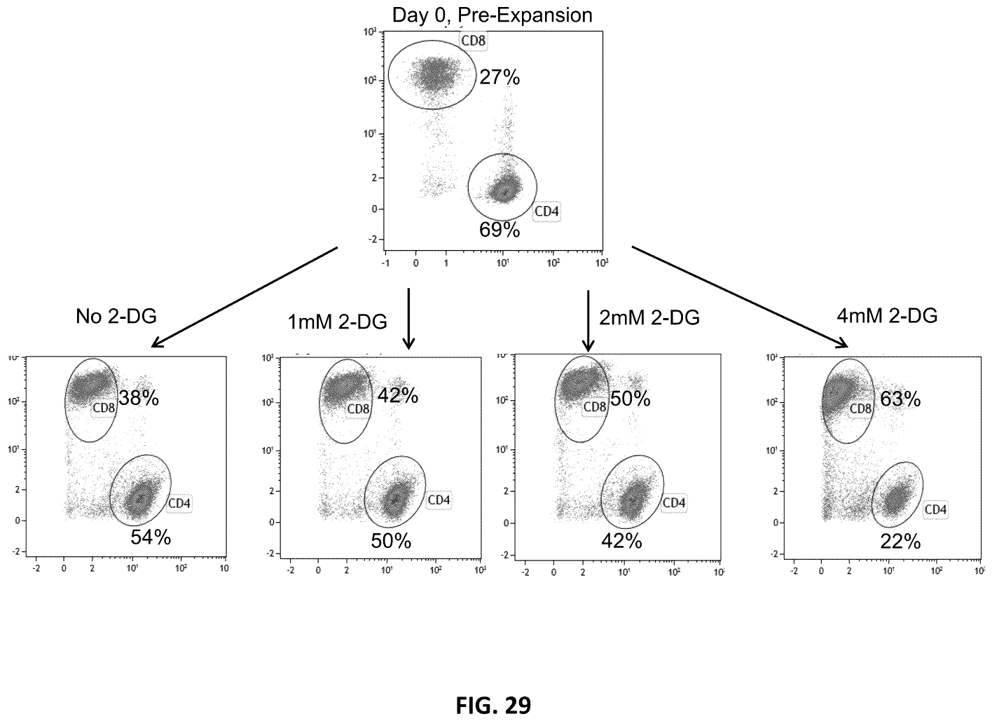

FIG. 29 depicts the gating strategy for differentiation phenotyping. At day 0, pre-expansion, the frequency of CD8+ T cells started at 27%. On day 10, post-expansion, without the addition of 2-DG, the CD8+ T cell population grew to 38%. However, with 4 mM 2-DG, the CD8+ T cell population increased to 63%.

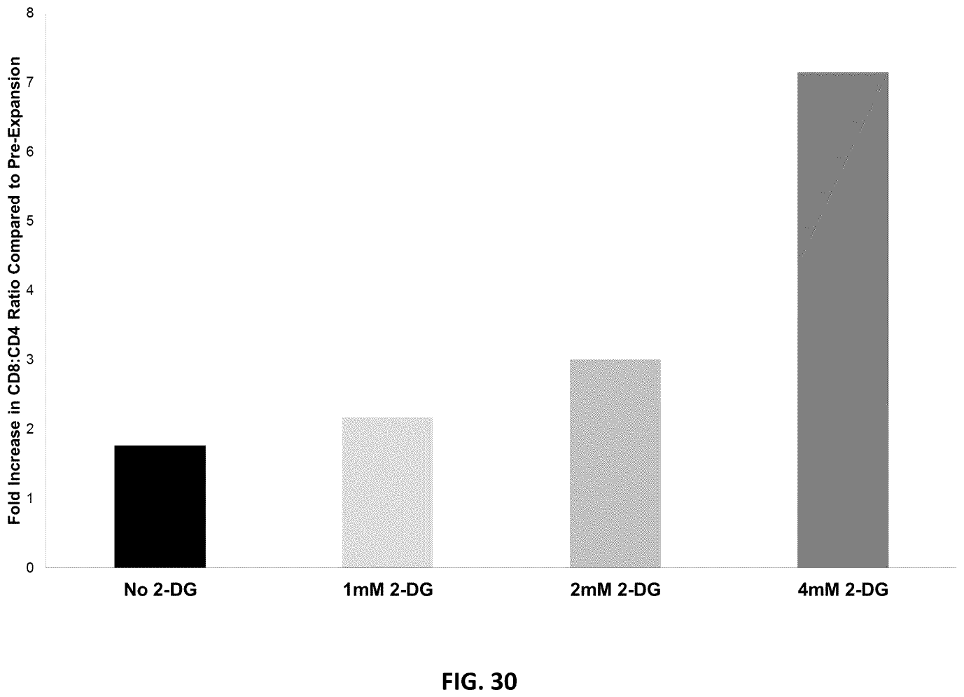

FIG. 30 depicts CD8+:CD4+ ratios compared to the original subset distribution prior to expansion (Day 0). Results show that 4 mM 2-DG results in a 4.1 fold increase in CD8:CD4 ratio compared to day 0, pre-expansion.

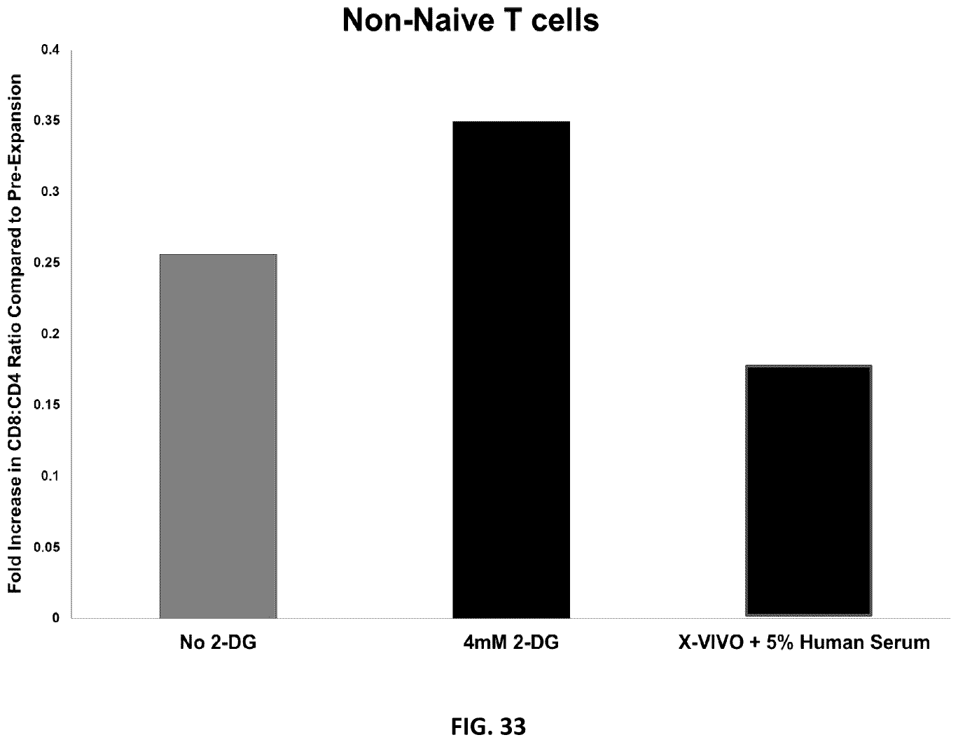

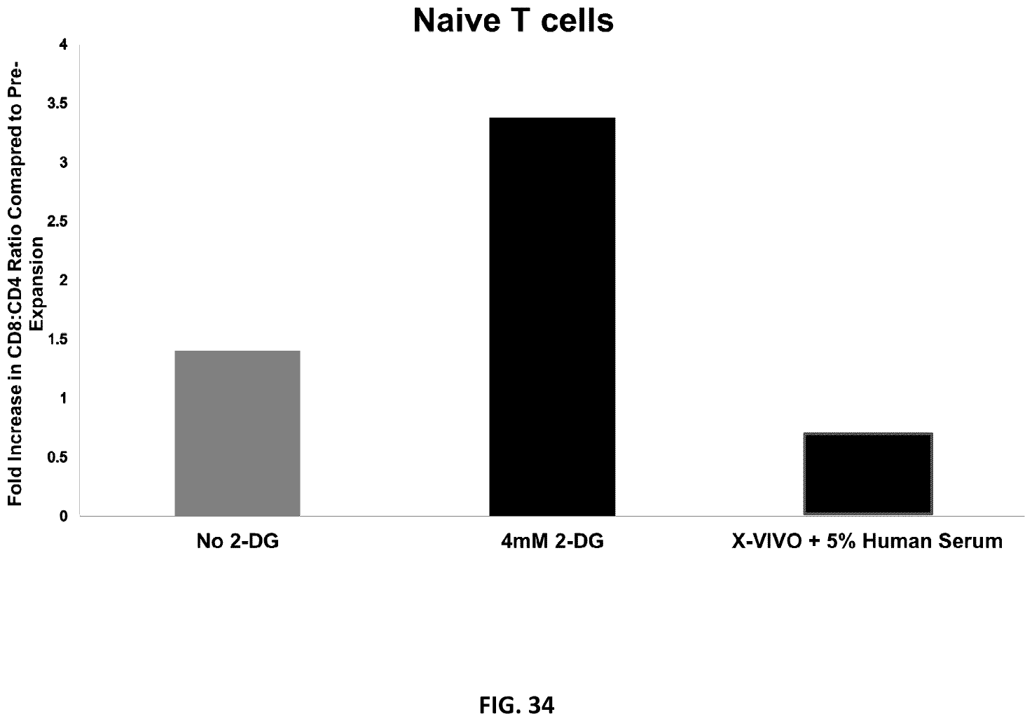

FIGS. 33 and 34 represent CD8+:CD4+ ratios compared to the original subset distribution prior to expansion (Day 0). Results demonstrate that there is a 2.4 fold increase in CD8:CD4 ratio in naïve T cells where there was a lesser effect in non-naïve T cells.

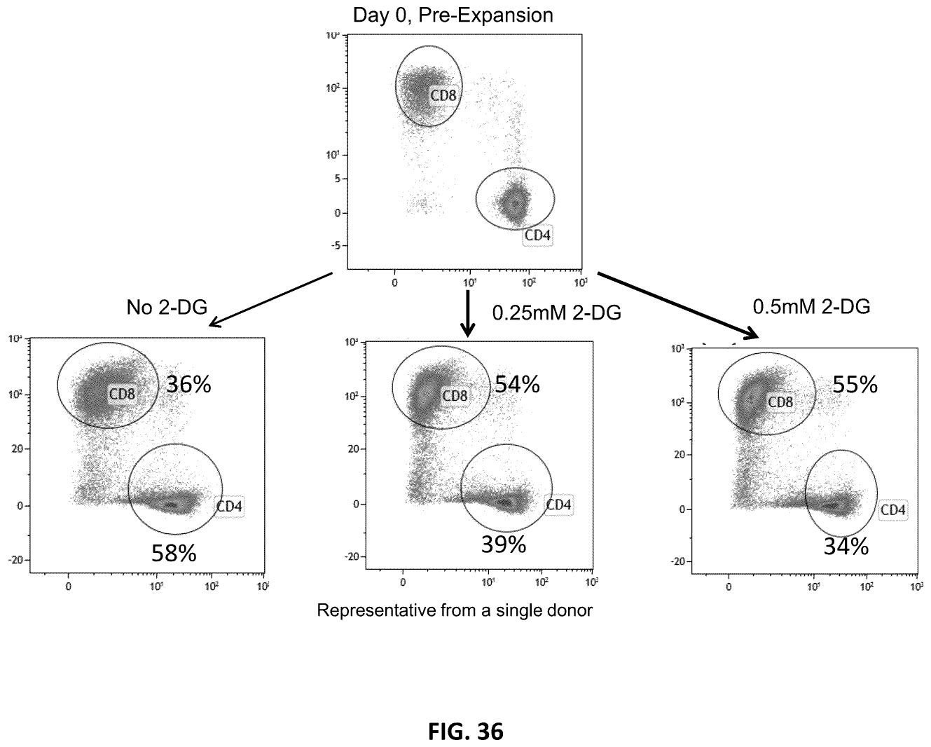

FIG. 36 depicts the gating strategy for differentiation phenotyping. At day 0, pre-expansion, the frequency of CD8+ T cells started at 19%. On day 10, post-expansion, without the addition of 2-DG, the CD8+ T cell population grew to 30%. However, with 0.25 mM and 0.5 mM 2-DG, the CD8+ T cell population increased to 48%. Representative from a single donor.

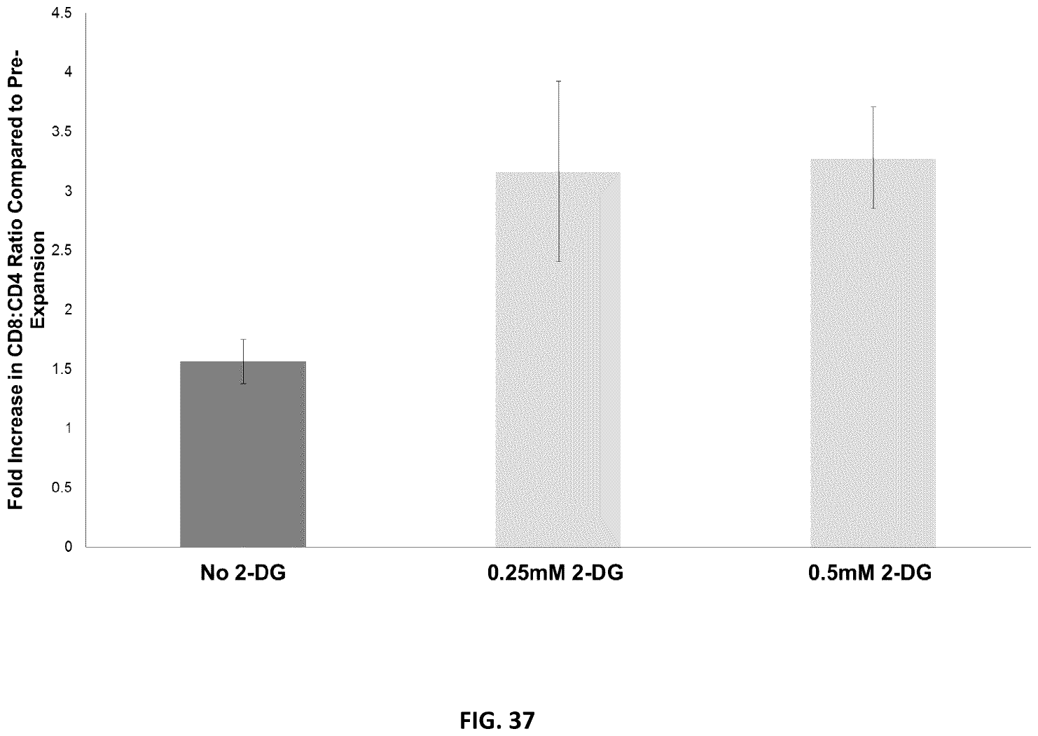

FIG. 37 depicts CD8+:CD4+ ratios compared to the original subset distribution prior to expansion (Day 0). Results show that there are a 2.1 and 2.2 fold increase in CD8:CD4 ratio with 0.25 mM and 0.5 mm 2-DG compared to day 0, pre-expansion. Representative from 3 different donors.

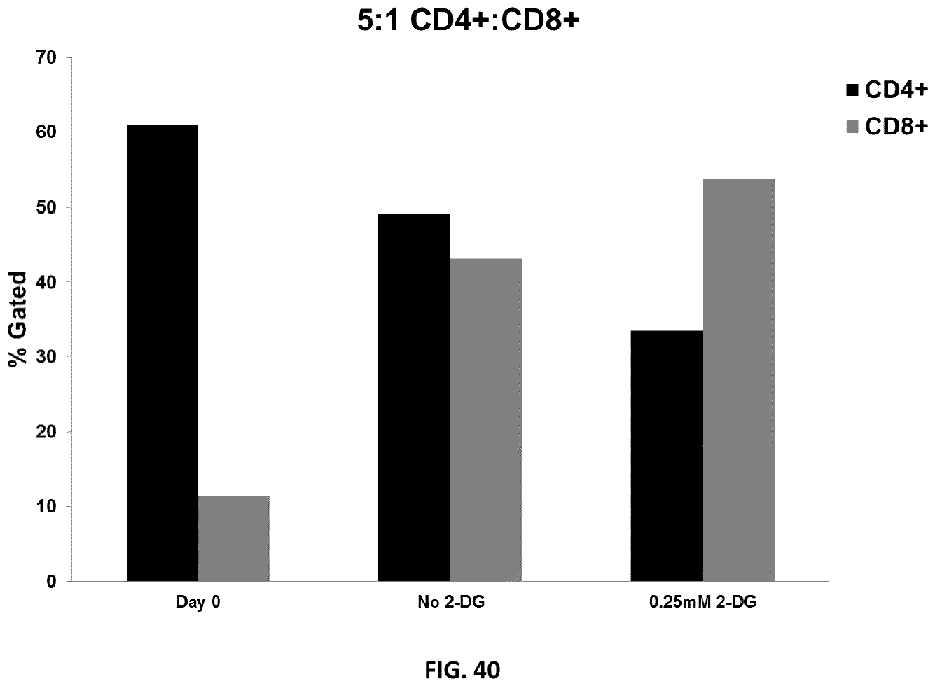

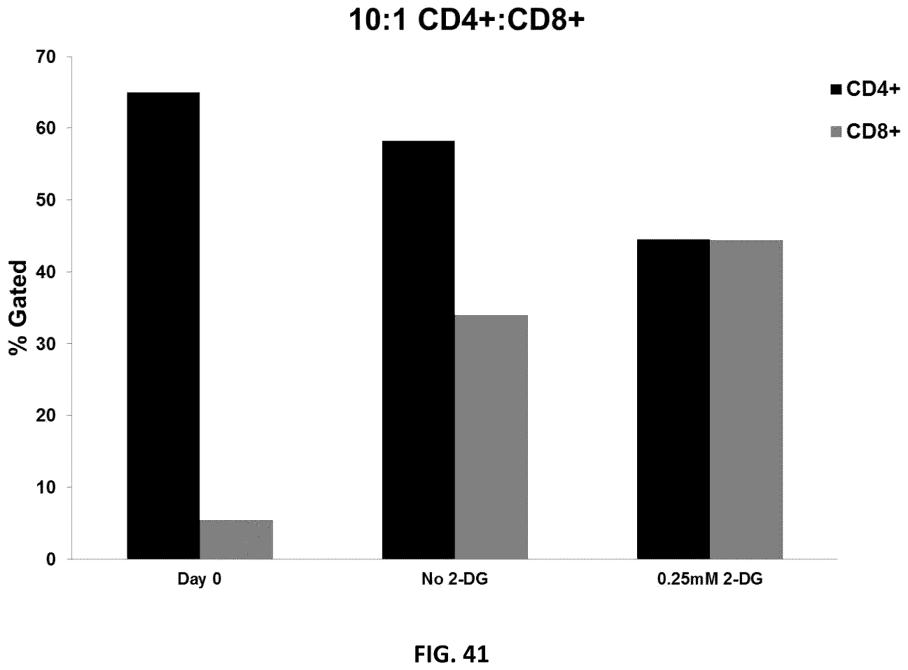

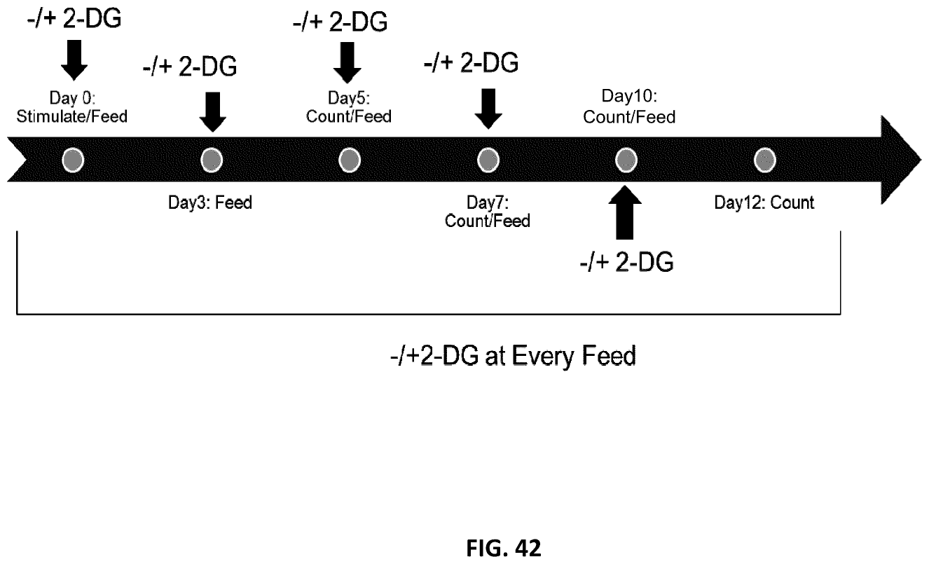

FIGS. 40 and 41 depict the average changes in CD4+/CD8+ ratios compared to the original subset distribution prior to expansion, day 0, which represents frequency of the two populations prior to expansion. 2-DG was able to correct large deficits in CD8+ T cells compared to day 0, pre-expansion.

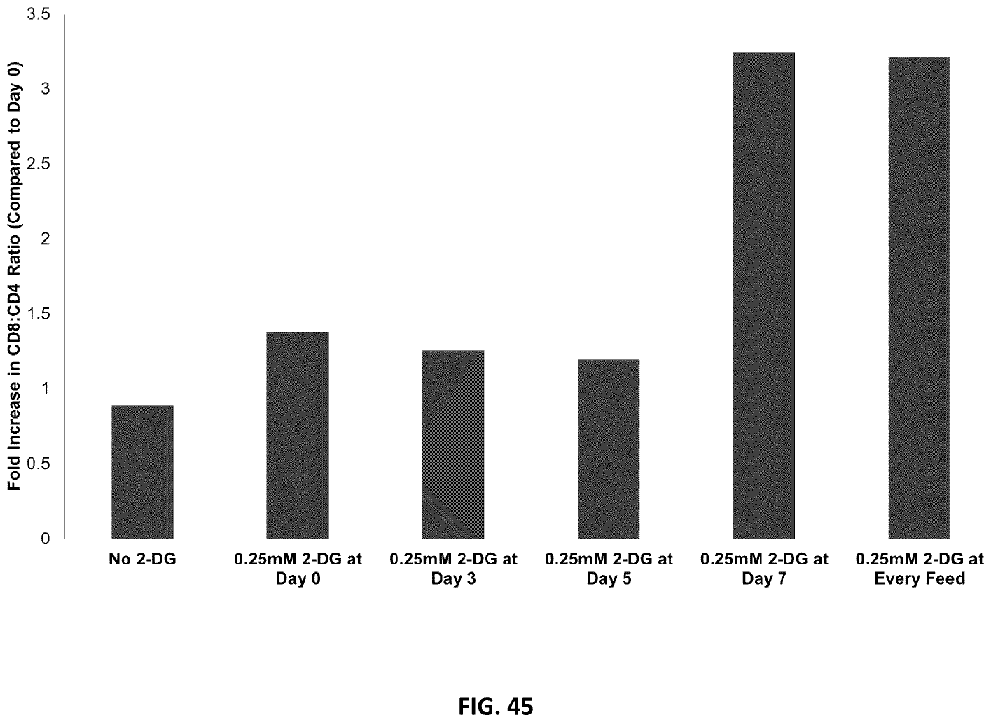

FIG. 45 represents the fold increase in CD8:CD4 ratio compared to day 0. Results show that culturing the cells with 0.25 mM 2-DG only on day 7 demonstrated the same results as culturing the cells with 2-DG at every feed and they both resulted in a 3 fold increase in CD8:CD4 ratio. This makes it easier to add 2-DG manipulation into our protocol as a tool for process development.

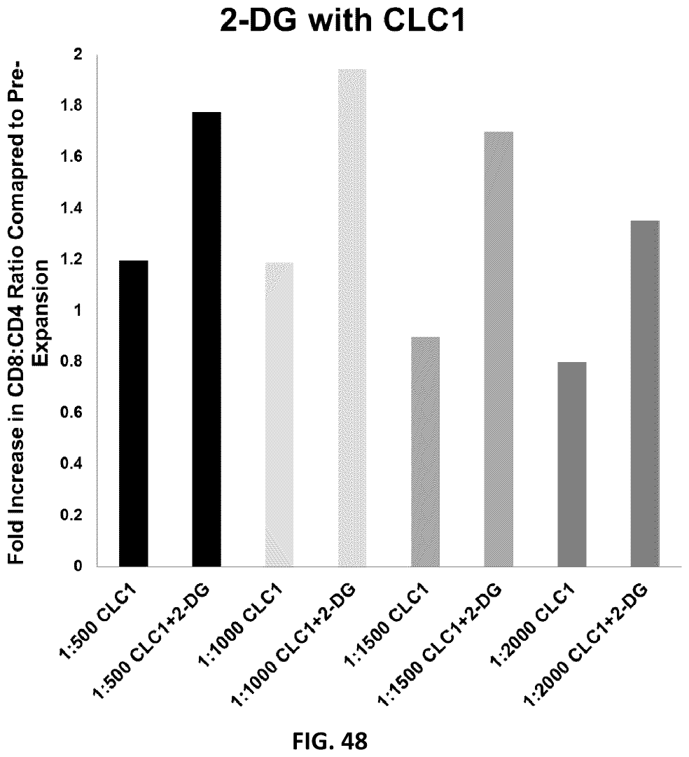

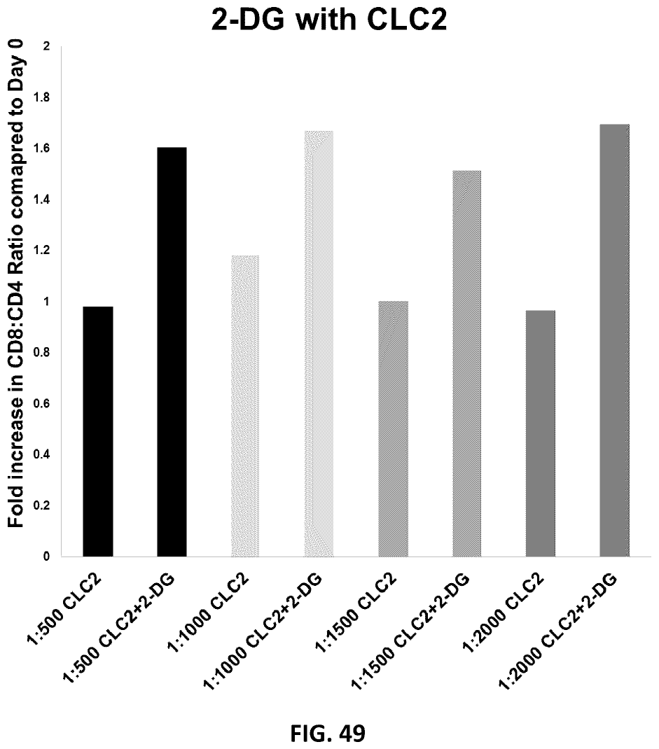

FIGS. 48 and 49 represent the fold increase in CD8:CD4 ratio compared to day 0. Results show that there is a 1.6 fold increase in CD8:CD4 ratio when adding 2-DG with CLC1 and a 1.4 fold increase in CD8:CD4 ratio when adding 2-DG with CLC2 compared to day 0, pre-expansion.

### Cytokine Profiles:

T cells were expanded for 12 days and re-stimulated with DYNABEADS® Human T-Expander CD3/CD28. Cytokine production upon re-stimulation was assessed with Invitrogen Cytokine Human Magnetic 35-Plex Panel for LUMINEX™. Results demonstrate that there is no change in cytokine production which means that 2-DG does not alter the function of the cells as measured by multiplexed cytokine assay as shown in (data not shown).

TABLE 25
Cultured T Cell Population Doublings with and without 2-DG
at different concentrations

| | | | | | X-VIVO ™ + |
|---|---|---|---|---|---|
| | No | 1 mM | 2 mM | 4 mM | 5% Human |
| Days | 2-DG | 2-DG | 2-DG | 2-DG | Serum |
| 0 | 0 | 0 | 0 | 0 | 0 |
| 5 | 2.02 | 2.08 | 1.87 | 1.92 | 2.09 |
| 7 | 3.56 | 3.87 | 3.53 | 3.95 | 2.52 |
| 10 | 6.98 | 7.19 | 7.10 | 7.50 | 5.56 |
| 12 | 6.92 | 7.00 | 6.90 | 7.35 | 6.62 |

TABLE 26
Fold Increase in CD8+ to CD4+ Ratios After 10 Days of Culture

| | | | | Fold Increase |
|---|---|---|---|---|
| | % | % | % Ratios | compared |
| Conditions | CD4+ | CD8+ | (CD8+/CD4+) | to Baseline |
| Day 0 | 69 | 27 | 0.39 | 1 |
| No 2-DG | 54 | 38 | 0.69 | 1.76 |
| (Day 10) | | | | |
| 1 mM 2-DG | 50 | 42 | 0.85 | 2.18 |
| (Day 10) | | | | |
| 2 mM 2-DG | 42 | 50 | 1.18 | 3.01 |
| (Day 10) | | | | |
| 4 mM 2-DG | 23 | 63 | 2.80 | 7.15 |
| (Day 10) | | | | |

TABLE 27
Cultured Non-Naïve T Cell Population Doublings
with and without 2-DG

| | | | X-Vivo ™ + |
|---|---|---|---|
| | No | 4 mM | 5% Human |
| Days | 2-DG | 2-DG | Serum |
| 0 | 0 | 0 | 0 |
| 5 | 0.54 | −0.24 | 0.69 |
| 7 | 2.19 | 1.25 | 2.27 |
| 10 | 4.05 | 3.37 | 4.64 |
| 12 | 4.84 | 4.64 | 6.02 |

TABLE 28
Cultured Naïve T Cell Population Doublings with and
without 2-DG

| | | | X-Vivo ™ + 5% |
|---|---|---|---|
| Days | No 2-DG | 4 mM 2-DG | Human Serum |
| 0 | 0 | 0 | 0 |
| 5 | 1.79 | 1.70 | 1.16 |
| 7 | 4.00 | 3.92 | 3.60 |
| 10 | 5.80 | 5.45 | 6.35 |
| 12 | 6.54 | 6.13 | 7.29 |

TABLE 29
Fold Increase in CD8+ to CD4+ Ratios After 10 Days of Culture in Non-Naive T Cells

| | | | | | | Fold Increase |
|---|---|---|---|---|---|---|
| | % | % | % CD4+ | % CD8+ | % Ratios | compared to |
| Conditions | CD4+ | CD8+ | (Avg) | (Avg) | (CD8+/CD4+) | Baseline |
| Day 0 | 64.79 | 27.92 | 64.79 | 27.92 | 0.43 | 1 |
| No 2-DG | 81.28 | 35.84 | 82.52 | 9.11 | 0.11 | 0.25 |
| (Day 10) | 83.75 | 54.69 | | | | |
| 4 mM 2-DG | 80.85 | 12.23 | 81.08 | 12.23 | 0.15 | 0.35 |
| (Day 10) | 81.31 | 12.23 | | | | |
| X-VIVO ™ + 5% | 79.02 | 5.94 | | | | |
| Human Serum | 80.27 | 6.05 | 79.65 | 5.99 | 0.07 | 0.17 |
| (Day 10) | | | | | | |

TABLE 30
Fold Increase in CD8+ to CD4+ Ratios After 10 Days of Culture in Naïve T Cells

| | | | | | | Fold Increase |
|---|---|---|---|---|---|---|
| | % | % | % CD4+ | % CD8+ | % Ratios | compared to |
| Conditions | CD4+ | CD8+ | (Avg) | (Avg) | (CD8+/CD4+) | Baseline |
| Day 0 | 59.69 | 36.66 | 59.69 | 36.66 | 0.61 | 1 |
| No 2-DG | 48.80 | 41.02 | 48.215 | 41.55 | 0.86 | 1.40 |
| (Day 10) | 47.63 | 42.02 | | | | |
| 4 mM 2-DG | 25.23 | 53.26 | 59.69 | 36.66 | 2.07 | 3.38 |
| (Day 10) | 25.82 | 52.71 | | | | |
| X-VIVO ™ + 5% | 61.78 | 25.88 | 61.08 | 27.67 | 0.45 | 0.74 |
| Human Serum | 60.638 | 29.46 | | | | |
| (Day 10) | | | | | | |

TABLE 31
Cultured T Cell Population Doublings with and without
0.25 mM and 0.5 mM 2-DG

| | | 0.25 mM 2-DG | 0.5 mM |
|---|---|---|---|
| Days | No 2-DG | (Avg/SD) | 2-DG |
| 0 | 0 | 0 | 0 |
| 5 | 1.57 | 1.64 | 1.69 |
| | 1.31 | 1.57 | 1.04 |
| | 0.63 | 1.44 | 1.40 |
| | (1.17/0.49) | (1.54/0.09) | (1.38/0.33) |
| 7 | 3.04 | 3.40 | 3.95 |
| | 3.20 | 3.54 | 3.32 |
| | 2.58 | 3.35 | 3.33 |
| | (2.94/0.32) | (3.43/0.10) | (3.50/0.39) |
| 10 | 4.78 | 4.85 | 5.06 |
| | 5.18 | 5.70 | 6.28 |
| | 4.50 | 5.81 | 6.75 |
| | (4.82/0.34) | (5.45/0.53) | (6.03/0.87) |
| 12 | 5.18 | 5.72 | 6.13 |
| | 5.86 | 6.57 | 6.22 |
| | 5.16 | 6.47 | 6.48 |
| | (5.40/0.40) | (6.26/0.46) | (6.28/0.18) |

TABLE 32
Fold Increase in CD8+ to CD4+ Ratios After 10 Days of Culture

| | | | | Fold | | Fold Increase |
|---|---|---|---|---|---|---|
| | | | | Increase | % Ratios | compared to |
| | % | % | % Ratios | compared | (CD8+/CD4+) | Baseline |
| Conditions | CD4+ | CD8+ | (CD8+/CD4+) | to Baseline | (Avg) | (Avg/SD) |
| Day 0 | 65.69 | 29.65 | 0.451362 | | 0.7 | 1 |
| | 54.89 | 42.99 | 0.783203 | | | |
| | 55 | 42.75 | 0.777273 | | | |
| No 2-DG | 57.84 | 35.84 | 0.61964 | 1.37 | 1.1 | 1.6/0.2 |
| (Day 10) | 40.1 | 54.69 | 1.36384 | 1.74 | | |
| | 41.42 | 50.76 | 1.23 | 1.58 | | |
| 0.25 mM 2-DG | 39.21 | 53.46 | 1.36 | 3.02 | 2.1 | 3.2/0.8 |
| (Day 10) | 32.39 | 63.2 | 1.95 | 2.49 | | |
| | 22.68 | 70.32 | 3.10 | 3.99 | | |
| 0.5 mM 2-DG | 37.08 | 55.04 | 1.48 | 3.29 | 2.2 | 3.6.0.4 |
| (Day 10) | 29.85 | 66.57 | 2.23 | 2.85 | | |
| | 23.85 | 68.64 | 2.88 | 3.7 | | |

TABLE 33
Cultured T Cell Population
Doublings at 5:1 CD4+:CD8+ Cells

| Days | No 2-DG | 0.25 mM 2-DG |
|---|---|---|
| 0 | 0 | 0 |
| 5 | 1.89 | 1.95 |
| 7 | 3.87 | 2.84 |
| 10 | 6.82 | 5.44 |
| 12 | 7.36 | 6.17 |

TABLE 34
Cultured T Cell Population
Doublings at 10:1 CD4+:CD8+ Cells

| Days | No 2-DG | 0.25 mM 2-DG |
|---|---|---|
| 0 | 0 | 0 |
| 5 | 2.06 | 1.97 |
| 7 | 3.98 | 2.98 |
| 10 | 6.87 | 5.67 |
| 12 | 7.42 | 6.25 |

TABLE 35
Changes in CD4+/CD8+ Ratios After 10 Days of
Culture (5:1 CD4+ CD8+ Ratio)

| Conditions | % CD4+ | % CD8+ | % CD4+ (Avg) | % CD8+ (Avg) |
|---|---|---|---|---|
| Day 0 | 60.88 | 11.42 | 60.88 | 11.42 |
| No 2-DG | 49.33 | 43.18 | 49.16 | 43.05 |
| (Day 10) | 48.99 | 42.92 | | |
| 0.25 mM | 32.91 | 53.50 | 33.39 | 53.83 |
| 2-DG | 33.88 | 54.17 | | |
| (Day 10) | | | | |

TABLE 36
Changes in CD4+/CD8+ Ratios After 10 Days of
Culture (10:1 CD4+ CD8+ Ratio)

| Conditions | % CD4+ | % CD8+ | % CD4+ (Avg) | % CD8+ (Avg) |
|---|---|---|---|---|
| Day 0 | 65.03 | 5.42 | 65.03 | 5.42 |
| No 2-DG | 57.88 | 33.92 | 58.195 | 33.97 |
| (Day 10) | 58.51 | 34.02 | | |
| 0.25 mM 2-DG | 44.25 | 45.35 | 44.57 | 44.45 |
| (Day 10) | 44.89 | 43.55 | | |

TABLE 37
Cultured T Cell Population Doublings with Culturing
2-DG at days 0, 3, 5, 7 and at every feed

| | | | | | | 0.25 mM | |
|---|---|---|---|---|---|---|---|
| | | 0.25 mM | 0.25 mM | 0.25 mM | 0.25 mM | 2-DG at | |
| | | 2-DG at | 2-DG at | 2-DG at | 2-DG at | Day at | 5% Human |
| Days | No 2-DG | Day 0 | Day 3 | Day 5 | Day 7 | every feed | Serum |
| 0 | 0 | 0 | 0 | 0 | 0 | 0 | 0 |
| 5 | 1.81 | 1.71 | 1.90 | 1.75 | 1.70 | 1.61 | 1.74 |
| 7 | 3.72 | 3.69 | 3.36 | 3.50 | 3.48 | 3.80 | 3.44 |
| 10 | 6.43 | 6.53 | 5.80 | 5.82 | 5.83 | 6.03 | 6.43 |
| 12 | 6.97 | 7.47 | 6.34 | 6.59 | 6.44 | 6.82 | 7.39 |

TABLE 38
Cultured T Cell Population Doublings with Culturing
2-DG at day 7 and at every feed

| | | 0.25 mM | 0.25 mM 2-DG | 5% |
|---|---|---|---|---|
| | | 2-DG | at Day at | Human |
| Days | No 2-DG | at Day 7 | every feed | Serum |
| 0 | 0 | 0 | 0 | 0 |
| 5 | 1.81 | 1.70 | 1.61 | 1.74 |
| 7 | 3.72 | 3.48 | 3.80 | 3.44 |
| 10 | 6.43 | 5.83 | 6.03 | 6.43 |
| 12 | 6.97 | 6.44 | 6.82 | 7.39 |

TABLE 39
Fold Increase in CD8+ to CD4+ Ratios of 2-DG at Different
Time Points and Every Feed After 10 Days of Culture

| | | | % Ratios | Fold Increase |
|---|---|---|---|---|
| | % | % | (CD8+/ | compared to |
| Conditions | CD4+ | CD8+ | CD4+) | Baseline |
| Day 0 | 78.4 | 17.41 | 0.22 | 1 |
| No 2-DG (Day 10) | 82.21 | 16.13 | 0.20 | 0.88 |
| 0.25 mM 2-DG at Day 0 | 74.78 | 22.88 | 0.31 | 1.38 |
| (Day 10) | | | | |
| 0.25 mM 2-DG at Day 3 | 70.62 | 25.76 | 0.36 | 1.64 |
| (Day 10) | | | | |
| 0.25 mM 2-DG at Day 5 | 76.18 | 21.26 | 0.28 | 1.26 |
| (Day 10) | | | | |
| 0.25 mM 2-DG at Day 7 | 73.28 | 23.91 | 0.33 | 1.47 |
| (Day 10) | | | | |
| 0.25 mM 2-DG at Every | 76.76 | 20.35 | 0.27 | 1.19 |
| Feed (Day 10) | | | | |

TABLE 40
Cultured T Cell Population Doublings with Culturing 2-DG with CD Lipid Concentrate 1

| | | | | | 1:500 | 1:1000 | 1:1500 | 1:2000 | |
|---|---|---|---|---|---|---|---|---|---|
| | 1:500 | 1:1000 | 1:1500 | 1:2000 | CLC1 + | CLC1 + | CLC1 + | CLC1 + | 5% Human |
| Days | CLC1 | CLC1 | CLC1 | CLC1 | 2-DG | 2-DG | 2-DG | 2-DG | AB Serum |
| 0 | 0 | 0 | 0 | 0 | 0 | 0 | 0 | 0 | 0 |
| 5 | 1.37 | 1.50 | 1.57 | 1.30 | 1.23 | 1.61 | 1.11 | 1.09 | 1.87 |
| 7 | 3.16 | 3.11 | 2.99 | 2.71 | 2.15 | 2.83 | 1.97 | 1.59 | 3.98 |
| 10 | 4.81 | 4.38 | 4.43 | 4.62 | 4.04 | 4.78 | 3.54 | 2.68 | 6.59 |
| 12 | 5.65 | 4.98 | 4.74 | 5.31 | 4.59 | 5.72 | 4.29 | 3.28 | 7.92 |

TABLE 41
Cultured T Cell Population Doublings with Culturing 2-DG with CD Lipid Concentrate 2

| | | | | | 1:500 | 1:1000 | 1:1500 | 1:2000 | |
|---|---|---|---|---|---|---|---|---|---|
| | 1:500 | 1:1000 | 1:1500 | 1:2000 | CLC2 + | CLC2 + | CLC2 + | CLC2 + | 5% Human |
| Days | CLC2 | CLC2 | CLC2 | CLC2 | 2-DG | 2-DG | 2-DG | 2-DG | AB Serum |
| 0 | 0 | 0 | 0 | 0 | 0 | 0 | 0 | 0 | 0 |
| 5 | 1.31 | 1.52 | 1.43 | 1.48 | 1.35 | 1.73 | 1.50 | 1.79 | 1.87 |
| 7 | 2.97 | 3.21 | 3.02 | 3.02 | 2.66 | 3.29 | 2.67 | 3.11 | 3.98 |
| 10 | 4.29 | 4.69 | 4.87 | 4.68 | 4.55 | 5.28 | 4.62 | 5.02 | 6.59 |
| 12 | 5.22 | 5.27 | 5.50 | 4.90 | 5.65 | 6.09 | 5.65 | 5.94 | 7.92 |

TABLE 42
Fold Increase in CD8+ to CD4+ Ratios of CLC1 with 2-DG

| | | | % Ratios | Fold Increase |
|---|---|---|---|---|
| | % | % | (CD8+/ | compared |
| Conditions | CD4+ | CD8+ | CD4+) | to Baseline |
| Day 0 | 69.51 | 25.34 | 0.36 | 1 |
| 1:500 (Day 10) | 61.8 | 26.9 | 0.4 | 1.2 |
| 1:1000 (Day 10) | 57.2 | 37.0 | 0.6 | 1.8 |
| 1:1500 (Day 10) | 55.1 | 23.9 | 0.4 | 1.2 |
| 1:2000 (Day 10) | 53.9 | 38.3 | 0.7 | 1.9 |
| 1:500 CLC1 + 2-DG | 54.6 | 17.9 | 0.3 | 0.9 |
| (Day 10) | | | | |
| 1:1000 CLC1 + 2-DG | 53.1 | 32.9 | 0.6 | 1.7 |
| (Day 10) | | | | |
| 1:1500 CLC1 + 2-DG | 72.3 | 21.0 | 0.3 | 0.8 |
| (Day 10) | | | | |
| 1:2000 CLC1 + 2-DG | 63.2 | 31.1 | 0.5 | 1.4 |
| (Day 10) | | | | |

TABLE 43
Fold Increase in CD8+ to CD4+ Ratios of CLC1 with 2-DG

| | | | % Ratios | Fold Increase |
|---|---|---|---|---|
| | % | % | (CD8+/ | compared |
| Conditions | CD4+ | CD8+ | CD4+) | to Baseline |
| Day 0 | 69.51 | 25.34 | 0.36 | 1 |
| 1:500 (Day 10) | 63.3 | 22.6 | 0.4 | 1.0 |
| 1:1000 (Day 10) | 55.6 | 32.5 | 0.6 | 1.6 |
| 1:1500 (Day 10) | 59.8 | 25.8 | 0.4 | 1.2 |
| 1:2000 (Day 10) | 58.2 | 35.5 | 0.6 | 1.7 |
| 1:500 CLC2 + 2-DG | 66.2 | 24.2 | 0.4 | 1.0 |
| (Day 10) | | | | |
| 1:1000 CLC2 + 2-DG | 61.5 | 33.9 | 0.6 | 1.5 |
| (Day 10) | | | | |
| 1:1500 CLC2 + 2-DG | 65.4 | 23.0 | 0.4 | 1.0 |
| (Day 10) | | | | |
| 1:2000 CLC2 + 2-DG | 58.4 | 36.1 | 0.6 | 1.7 |
| (Day 10) | | | | |

### Example 8: Culturing Diploid Cells for Vaccine Production in Serum-Free Medium (SFM) Containing Lipids

### Background

Lipids, traditionally supplied by fetal bovine serum, also appear necessary for diploid cells grown in serum-free medium and for optimal diploid cell expansion.

Popularly, diploid cells are used for vaccine production, which may involve production of the whole virus, or part of the virus, viral particles, viral proteins, viral DNA, or fragments thereof. Examples of viruses that have been produced for vaccines include but are not limited to, Varicella zoster (VZV), Rubella, Measles, MMR, Hepatitis A, Adenovirus, Poliomyelitis, Rotavirus, Rabies or vesicular stomatitis virus (VSV), Dengue virus, etc. Cells used for vaccine production include human diploid cells like MRC-5, MRC-5 RCB, WI-38, 2BS, Walvax-2, KMB-17, IMR-90, IMR-91, etc., other proprietary diploid cell lines used for in-house vaccine manufacture, and non-human diploid cells like VERO (African Green Monkey Kidney) cells, and so on.

Vaccine manufacturers generally culture diploid cells in classical media that contain 10% bovine serum and desire to move to serum-free formulations due to the potential regulatory and supply chain risks associated with serum. Using metabolite analysis and a design of experiment (DOE) rationale, we have developed the first serum-free medium (SFM) for the ‘growth’ of diploid cells, which we also refer to as Diploid Growth SFM The advantages of using the Diploid Growth SFM is that it is serum-free, it supports the direct recovery of cells from thaw to adaptation-free expansion, it can support the recovery of diploid cells frozen previously in serum-containing medium, all while resulting in performances that are comparable to serum containing medium Since the requirements for the production of viruses are different from that of cell growth, diploid production medium was optimized separately, and that resulted in the making of an animal origin-free (AOF) Diploid Production SFM. Together, the Diploid Growth SFM and the AOF Diploid Production SFM, give manufacturers the opportunity to produce vaccines without the concern of the presence of bovine serum albumin (BSA), thereby fulfilling the 50 ng/dose BSA limit set by the WHO. By switching to a serum-free process, vaccine manufacturers can reduce their dependency on serum, reduce their production and purification costs, and increase their product consistency and safety.

### Materials and Methods

The compositions and methodology for preparing the CD cyclodextrin-lipid supplements described below are the same as those described above under Example 1: CD Supplement Formulations. The Diploid Growth Supplement (Thermo Fisher Scientific, Cat. No. A39695SA) was prepared with or without CD cyclodextrin-lipid supplement, as described below.

### 1. Cell Culture

MRC-5 pd19 cells (ECACC, Cat. No. 05072101) were thawed into Diploid Basal SFM (Thermo Fisher Scientific, Cat. No. A39693DK) containing 6 mM L-Glutamine and 1% Diploid Growth Supplement—with or without CD lipid supplement. For the controls, the MRC-5 pd19 cells were thawed into MEM Alpha medium (Thermo Fisher Scientific, Cat. No. 12571-063) supplemented with 10% Fetal Bovine Serum (Thermo Fisher Scientific, Cat. No. 10082), 4 mM L-Glutamine, and 2 g/L D-Glucose. The cells were washed with Dulbecco's Phosphate-Buffered Saline (Thermo Fisher Scientific, Cat. No. 14190-136), dissociated with Trypsin-EDTA (0.05%) (Thermo Fisher Scientific, Cat. No. 25300-054), quenched with 2× Defined Trypsin Inhibitor (Thermo Fisher Scientific, Cat. No. R-007-100), and passaged every 3-4 days. Cell counts were performed using a Vi-CELL XR analyzer (Beckman Coulter, Indianapolis, IN) and seeded at a density of 0.6×10⁶ VCD in 25 mL growth medium for 3 day culture in a vented T-flask 75 cm² (Corning, Cat. No. 353136) or seeded at a density of 0.3×10⁶ VCD in 25 mL growth medium for 4 day culture in a vented T-flask 75 cm² (Corning, Cat. No. 353136).

### 2. Virus Production

For virus production (e.g., varicella zoster virus production), exemplary diploid cells, e.g., MRC-5, were seeded at 40,000 cells/cm², either in Diploid Basal SFM (Thermo Fisher Scientific, Cat. No. A39693DK) containing 6 mM L-Glutamine and 1% Diploid Production (Thermo Fisher Scientific, Cat. No. A39696SA), or, for the control: into MEM Alpha medium supplemented with 10% Fetal Bovine Serum (Thermo Fisher Scientific, Cat. No. 10082), 4 mM L-Glutamine, and 2 g/L D-Glucose. After 24 hours, the media was exchanged to Diploid SFM containing 6 mM L-Glutamine, or MEM Alpha with 2% FBS, 4 mM L-Glutamine, and 2 g/L D-Glucose, respectively. Cells were infected with varicella zoster virus (ATCC® VR-1367™) at an MOI of 0.01 and harvested after 2 days. Virus titers were determined by TCID₅₀ assay.

### Results

### Cell Growth and Viability Measurements

Diploid viable cell density (VCD) is expressed as viable cells per milliliter and is shown as a % control of each cell line in Diploid Growth SFM versus serum-containing medium control (MEM Alpha+10% FBS), and these results are representative of at least 3 independent experiments.

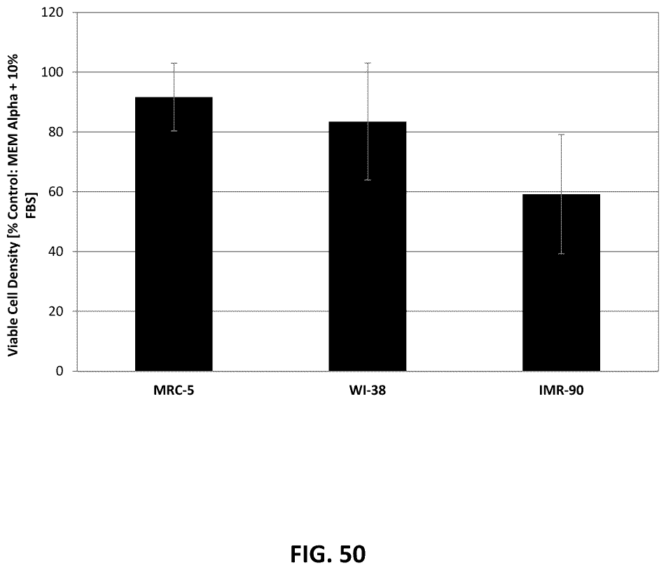

FIG. 50 and Table 44 below depict the average % (VCD) for MRC-5, WI-38, and IMR-90 cell lines over 5 passages compared to the growth of each cell line in the control MEM Alpha medium+10% FBS. The results indicate that for MRC-5 cells, the growth is almost comparable to that of the serum-containing medium control.

TABLE 44
Viable Cell Density of Diploid Cells in Diploid SFM Compared to each
cell line grown in MEM Alpha + 10% FBS (Average Over 4 Passages)

| Cell Line | % Control (VCD) | Standard Deviation |
|---|---|---|
| MRC-5 | 91.664 | 11.294 |
| WI-38 | 83.462 | 19.562 |
| IMR-90 | 59.181 | 19.945 |

FIG. 51 and Table 45 below depict a comparison of VCD for MRC-5 cells in Diploid Growth SFM with or without lipid supplementation compared to MRC-5 cells grown in control MEM Alpha medium. Further, 2 types of lipid supplement comparisons were made: 1) in “Lipid Concentrate” (1:100 and 1:1000) (Thermo Fisher Scientific, cat. no. 11905-031), and 2) in CD Supplements 1 and 2 (1:2000 and 1:500 each) whose preparation is described above in Example 1. Results indicate that CD Supplements 1 and 2 increase MRC-5 VCD compared to Lipid Concentrate (1:100 and 1:1000). Additionally, results also indicate that CD (cyclodextrin-based) Supplements 1 and 2 increased MRC-5 VCD comparable to VCDs in serum-containing medium (FIG. 51 and Table 45).

Hence the Diploid SFM containing CD lipid supplement yields diploid cell growth comparable or superior to that of serum-containing medium.

TABLE 45
Viable Cell Density of MRC-5 Cells in Diploid
SFM with Different Lipid Supplements Compared to
MEM Alpha + 10% FBS (Average Over 4 Passages)

| Medium # | % Control (VCD) | Standard Deviation |
|---|---|---|
| MEMα + 10% FBS | 100.000 | 0 |
| Lipid Concentrate (1:1000) | 29.1 | 4.75 |
| Lipid Concentrate (1:100) | 45.9 | 3.16 |
| CD Sup. 1 (1:2000) | 86.83 | 19.61 |
| CD Sup. 1 (1:500) | 103.0 | 21.22 |
| CD Sup. 2 (1:2000) | 92.5 | 11.10 |
| CD Sup. 2 (1:500) | 107.3 | 25.89 |

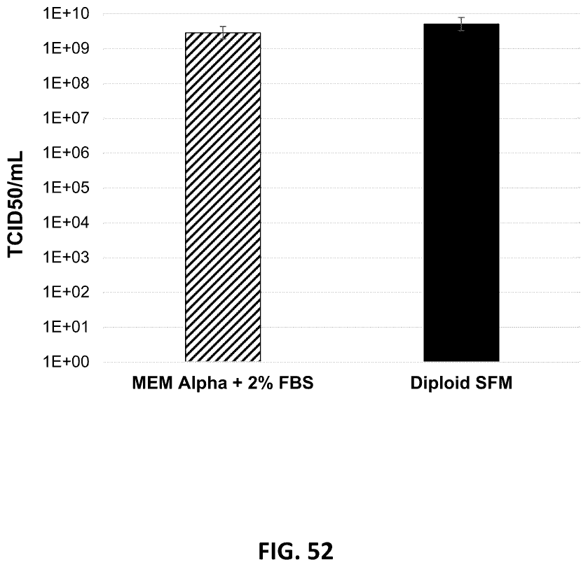

FIG. 52 and Table 46 below demonstrates that MRC-5 cells grown in Diploid SFM yields vancella zoster virus (VZV) production comparable to that of cells grown in serum-containing medium.

TABLE 46
Varicella Zoster Virus Production with MRC-5 Cells in Diploid SFM

| Medium | TCID50/mL | Std. Dev + | Std. Dev |
|---|---|---|---|
| MEM Alpha + 2% FBS | 2.81E+09 | 1.47E+09 | 9.67E+08 |
| Diploid SFM | 5.00E+09 | 2.73E+09 | 1.76E+09 |

Besides culturing human diploid cells (as shown above), the Diploid Growth SFM comprising diploid growth supplements CD1 or CD2 were also capable of culturing non-human diploid cells like VERO cells (data not shown).

### Kits for Growing Cells for Virus Production Under SFM Conditions

Exemplary kits for the culturing cells for vaccine production, including diploid and non-diploid cells may contain the following components shown in Kits 1 and 2:

Kit 1 consists of 1 L Diploid Basal Medium+10 mL Diploid Growth Supplement OR 10 L Diploid Basal Medium+100 mL Diploid Growth Supplement.

Kit 2 consists of 1 L Diploid Basal Medium+10 mL Diploid Production Supplement OR 10 L Diploid Basal Medium+100 mL Diploid Production Supplement.

In some embodiments, only the Diploid Growth Supplement may contain the cyclodextrin lipids, while the Diploid Basal Medium and the Diploid Production Supplement do not contain cyclodextrin and/or lipids.

These following kits for culturing vaccine producing cells are available from Thermo Fisher Scientific.

### Liquid Kit 1

Liquid Kit 1

| Kit SKU | Parent SKU | Size | | Product Name |
|---|---|---|---|---|
| A3968701 | Growth Supplement | 10 | mL | Diploid Growth |
| | Diploid Basal Medium | 1000 | mL | SFM Kit |
| A3968702 | Growth Supplement | 100 | mL | Diploid Growth |
| | Diploid Basal Medium | 10 | L | SFM Kit |

### Liquid Kit 2

Liquid Kit 2

| Kit SKU | Parent SKU | Size | | Product Name |
|---|---|---|---|---|
| A3968801 | Production Supplement | 10 | mL | Diploid |
| | | | | Production |
| | Diploid Basal Medium | 1000 | mL | SFM Kit |
| A3968802 | Production Supplement | 100 | mL | Diploid |
| | | | | Production |
| | Diploid Basal Medium | 10 | L | SFM Kit |

In another embodiment, both the Diploid Growth Supplement and the Diploid Production Supplement may contain cyclodextrin and/or lipids.

Some Applications where Diploid Cells Cultured Under SFM Conditions are Used

1. For virus/vaccine production, using methods and/or protocols described in the following references, all of which are hereby incorporated by reference.
a) Mirchamsy et al., Arch. Inst. Razi, (1979) 31, 97-102. Production of Measles and Rubella virus vaccines,
b) Rabies Vaccine Imovax® Rabies by Sanofi Pasteur, April (2013) v0.2 https://www.fda.gov/downloads/biologicsbloodvaccines/vaccines/approvedproducts/ucm133484.pdf.
c) Liu et al., “High Genetic Stability of Dengue Virus Propagated in MRC-5 Cells as Compared to the Virus Propagated in Vero Cells,” 3:e1810 (2008).

2. For testing the efficacy of vaccine production, or for determining virus titer, using methods and/or protocols described in, e.g., Hong, Virology Journal (2015) 12:101, hereby incorporated by reference.

3. For testing: i) virucide; ii) adventitious agents through detection of cytopathic effects on indicator cell lines such as MRC-5 cells, Vero cells, etc. (see, e.g., http://www.mds-usa.com/cellcharacter_adventitious.html) hereby incorporated by reference.

4. For transfection with a suitable transfection reagent (see for e.g., http://www.merckmillipore.com/INTERSHOP/web/WFS/Merck-JP-Site/ja_JP/-/JPY/ShowDocument-Pronet?id=201708.146) hereby incorporated by reference.

5. For diagnostic testing (see, e.g., CMV: Gregory et al., JOURNAL OF CLINICAL MICROBIOLOGY, 17:605-609 (1983), CMV, Rabies, Herpes Simplex, VSV, Echo, Rhinovirus (Dilnessa et al., Journal of Microbiology and Modern Techniques, 2:102 (2007)) hereby incorporated by reference).

6. In cell models (e.g., lung cancer model (Zuchowska et al., Biomicrofluidics 11:024110 (2017); hereby incorporated by reference)).

7. To study chromosomal instability (see Conry et al., PNAS 107:15455-15460 (2010)); or to study replicative senescence (see https://www.activemotif.com/catalog/details/40310/wi-38-nuclear-extract), both hereby incorporated by reference.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. GenBank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

Exemplary Subject Matter of the Invention is represented by the following clauses:

Clause 1. A combination comprising (i) a population of T cells and (ii) a cell culture medium that comprises a cyclodextrin and at least one lipid.

Clause 2. The combination of clause 1, wherein the at least one lipid is cholesterol, a fatty acid, a fatty acid ester, a phospholipid, or a glycerolipid.

Clause 3. The combination of clause 2, wherein the fatty acid is a saturated fatty acid, a monounsaturated fatty acid, or a polyunsaturated fatty acid.

Clause 4. The combination clause 2 or 3, wherein the fatty acid is an omega-3 fatty acid, an omega-6 fatty acid, or an omega-9 fatty acid.

Clause 5. The combination of any one of clauses 2-4, wherein the fatty acid is linoleic acid.

Clause 6. The combination of clause 5, further comprising linolenic acid.

Clause 7. The combination of clause 6, wherein the linolenic acid is alpha-linolenic acid, gamma-linolenic acid, or alpha-linolenic acid and gamma-linolenic acid.

Clause 8. The combination of any one of clauses 5-7, further comprising arachidonic acid.

Clause 9. The combination of any one of clauses 5-8, further comprising myristic acid, oleic acid, palmitic acid, palmitoleic acid, or stearic acid.

Clause 10. The combination of any one of clauses 1-9, wherein the at least one lipid is at least 2, 3, 4, 5, 6, 7, or 8 different fatty acids.

Clause 11. The combination of any one of clauses 1-10, wherein the at least one lipid is 2, 3, 4, 5, 6, 7, or 8 different fatty acids.

Clause 12. The combination of any one of clauses 1-11, wherein the at least one lipid is
(a) any one of linoleic acid, linolenic acid, arachidonic acid, myristic acid, oleic acid, palmitic acid, palmitoleic acid, or stearic acid;
(b) 2, 3, 4, 5, 6, or 7 of linoleic acid, linolenic acid, arachidonic acid, myristic acid, oleic acid, palmitic acid, palmitoleic acid, and stearic acid;
(c) linoleic acid, linolenic acid, arachidonic acid, myristic acid, oleic acid, palmitic acid, palmitoleic acid, and stearic acid;
(d) a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid;
(e) monounsaturated fatty acid, and the monounsaturated fatty acid is palmitoleic acid, oleic acid, eicosenoic acid, erucic acid, or nervonic acid;
(f) polyunsaturated fatty acid, and the polyunsaturated fatty acid is hexadecatrienoic acid, linoleic acid, alpha-linolenic acid, gamma-linolenic acid, stearidonic acid, eicosadienoic acid, eicosatrienoic acid, dihomo-gamma-linolenic acid, mead acid, arachidonic acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid;
(g) omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid;
(h) omega-6 fatty acid, and the omega-6 fatty acid is linoleic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid; or
(i) omega-9 fatty acid, and the omega-9 fatty acid is palmitoleic acid, oleic acid, eicosenoic acid, erucic acid, nervonic acid, or mead acid.

Clause 13. The combination of clause 1 or 2, wherein the at least one lipid is cholesterol.

Clause 14. The combination of clause 13, wherein the cholesterol is synthetic cholesterol.

Clause 15. The combination of any one of clauses 1-14, wherein the cyclodextrin is an α-cyclodextrin, a β-cyclodextrin, or a γ-cyclodextrin.

Clause 16. The combination of any one of clauses 1-15, wherein the cyclodextrin is methylated.

Clause 17. The combination of clause 16, wherein the cyclodextrin is methyl-β-cyclodextrin.

Clause 18. The combination of any one of clauses 1-16, wherein the cyclodextrin is 2-hydroxypropyl-β-cyclodextrin, sulfobutylether-β-cyclodextrin, 2-hydroxypropyl-γ-cyclodextrin, 2,6-dimethyl-α-cyclodextrin, hydroxypropyl-γ-cyclodextrin, hydroxyethyl-β-cyclodextrin, β-cyclodextrin polysulfate, trimethyl β-cyclodextrin, or γ-cyclodextrin polysulfate.

Clause 19. The combination of any one of clauses 1-18, wherein the composition comprises a plurality of different cyclodextrins, wherein the plurality of cyclodextrins comprises at least two cyclodextrins.

Clause 20. The combination of clause 19, wherein the plurality of cyclodextrins comprises at least one α-cyclodextrin, at least one β-cyclodextrin, and/or at least one γ-cyclodextrin.

Clause 21. The combination of any one of clauses 1-20, comprising linoleic acid, cholesterol, and the cyclodextrin.

Clause 22. The combination of any one of clauses 1-21, comprising the cyclodextrin and cholesterol, wherein the molar ratio of the cyclodextrin to the cholesterol is less than 10.5:1.

Clause 23. The combination of any one of clauses 1-21, comprising the cyclodextrin and at least one fatty acid, wherein the molar ratio of the cyclodextrin to the at least one fatty acid is less than 11.5:1.

Clause 24. The combination of any one of clauses 1-21, comprising the cyclodextrin, cholesterol, and at least one fatty acid, wherein the molar ratio of the cyclodextrin to the cholesterol and the at least one fatty acid is less than 7.5:1.

Clause 25. The combination of any one of clauses 1-21, comprising the cyclodextrin, cholesterol, and at least one fatty acid, wherein the molar ratio of the cyclodextrin to the cholesterol and the at least one fatty acid is less than 5.5:1.

Clause 26. The combination of any one of clauses 1-21, comprising the cyclodextrin, cholesterol, and at least one fatty acid, wherein the molar ratio of the cyclodextrin to the cholesterol and the at least one fatty acid is less than 4.5:1.

Clause 27. The combination of any one of clauses 1-26, further comprising a prostaglandin, a corticosteroid, a leukotriene, a lipoxin, a protectin, a resolvin, an oligonucleotide, or hydrophobic drug compound.

Clause 28. The combination of clause 27, wherein the hydrophobic drug compound is etomoxir or a statin.

Clause 29. The combination of any one of clauses 1-28, wherein the cell culture medium comprises a level of cyclodextrin that is less than about 200 μM.

Clause 30. The combination of any one of clauses 1-28, wherein the cell culture medium comprises a level of cyclodextrin that is from about 50 μM to about 200 μM.

Clause 31. The combination of any one of clauses 1-30, wherein the cell culture medium comprises a level of cholesterol that is from about 10 μM to about 30 μM.

Clause 32. The combination of any one of clauses 1-31, wherein the level of the at least one lipid in the cell culture medium is from about 10 μM to about 30 μM.

Clause 33. The combination of any one of clauses 1-26 or 29-32, which does not comprise a drug compound.

Clause 34. The combination of any one of clauses 1-26 or 29-32, which does not comprise alprostadil, cefotiam hexetil HCl, benexate HCl, dexamethasone, iodine, nicotine, nimesulide, nitroglycerin, omeprazol, PGE2, piroxicam, tiaprofenic acid, cisapride, hydrocortisone, ludomethacin, itraconazole, mitomycin, 17β-estradiol, chloramphenicol, voriconazole, ziprasidoue maleate, diclofenac sodium, etomoxir or a statin.

Clause 35. The combination of any one of clauses 1-26 or 29-32, which does not comprise a hydrophobic drug compound.

Clause 36. The combination of any one of clauses 1-35, wherein (i) the cell culture medium comprises albumin; or (ii) the cell culture medium does not comprise albumin.

Clause 37. The combination of any one of clauses 1-36, wherein (i) the cell culture medium comprises albumin; or (ii) the cell culture medium does not comprise a protein.

Clause 38. The combination of any one of clauses 1-37, wherein the cell culture medium is serum-free cell culture medium.

Clause 39. The combination of any one of clauses 1-38, wherein the population of T cells comprises T cells that are capable of greater retention of phenotype, greater expansion, greater potency, and/or higher transduction efficiency compared to corresponding T cells in a population of T cells that is in combination with a cell culture medium that does not comprise a cyclodextrin and at least one lipid.

Clause 40. The combination of any one of clauses 1-39, further comprising 2-deoxy-D-glucose.

Clause 41. A cell culture plate or flask, bag, biofermentor, or bioreactor system comprising the combination of any one of clauses 1-40.

Clause 42. A serum-free cell culture medium composition comprising linoleic acid, at least one other omega-6 fatty acid, cholesterol, and a methylated cyclodextrin.

Clause 43. The composition of clause 42, wherein the methylated cyclodextrin is present at a level from about 50 μM to about 200 μM.

Clause 44. The composition of clause 42 or 43, wherein the cholesterol is present at a level from about 10 μM to about 30 μM.

Clause 45. A serum-free cell culture supplement composition, comprising linoleic acid, at least one other omega-6 fatty acid, cholesterol, and a methylated cyclodextrin.

Clause 46. The composition of any one of clauses 42-45, herein the at least one other omega-6 fatty acid is a polyunsaturated omega-6 fatty acid.

Clause 47. The composition of any one of clauses 42-46, herein the at least one other omega-6 fatty acid is gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid.

Clause 48. The composition of any one of clauses 42-47, herein the at least one other omega-6 fatty acid is arachidonic acid.

Clause 49. The composition of any one of clauses 42-48, further comprising alpha-linolenic acid.

Clause 50. The composition of any one of clauses 42-49, further comprising myristic acid, oleic acid, palmitic acid, palmitoleic acid, or stearic acid.

Clause 51. The composition of any one of clauses 42-50, comprising at least 3, 4, 5, 6, 7, or 8 different fatty acids.

Clause 52. The composition of any one of clauses 42-50, comprising 3, 4, 5, 6, 7, or 8 different fatty acids.

Clause 53. The composition of any one of clauses 42-52, comprising
(a) any one of linolenic acid, arachidonic acid, myristic acid, oleic acid, palmitic acid, palmitoleic acid, or stearic acid;
(b) 2, 3, 4, 5, 6, or 7 of linolenic acid, arachidonic acid, myristic acid, oleic acid, palmitic acid, palmitoleic acid, and stearic acid;
(c) linolenic acid, arachidonic acid, myristic acid, oleic acid, palmitic acid, palmitoleic acid, and stearic acid;
(d) a saturated fatty acid, and the saturated fatty acid is butyric acid, caprylic acid, capric acid, lauric acid, myristic acid, palmitic acid, stearic acid, arachidic acid, behenic acid, lignoceric acid, or cerotic acid;
(e) monounsaturated fatty acid, and the monounsaturated fatty acid is palmitoleic acid, oleic acid, eicosenoic acid, erucic acid, or nervonic acid;
(f) polyunsaturated fatty acid, and the polyunsaturated fatty acid is hexadecatrienoic acid, alpha-linolenic acid, gamma-linolenic acid, stearidonic acid, eicosadienoic acid, eicosatrienoic acid, dihomo-gamma-linolenic acid, mead acid, eicosatetraenoic acid, eicosapentaenoic acid, heneicosapentaenoic acid, docosadienoic acid, adrenic acid, docosapentaenoic acid, docosapentaenoic acid, docosahexaenoic acid, tetracosatetraenoic acid, tetracosapentaenoic acid, tetracosapentaenoic acid, or tetracosahexaenoic acid;
(g) omega-3 fatty acid, and the omega-3 fatty acid is hexadecatrienoic, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, or tetracosahexaenoic acid;
(h) omega-6 fatty acid, and the omega-6 fatty acid is linoleic acid, arachidonic acid, gamma linolenic acid, eicosadienoic acid, dihomo-gamma-linolenic acid, arachidonic acid, docosadienoic acid, adrenic acid, docosapentaenoic acid, tetracosatetraenoic acid, or tetracosapentaenoic acid; or
(i) omega-9 fatty acid, and the omega-9 fatty acid is palmitoleic acid, oleic acid, eicosenoic acid, erucic acid, nervonic acid, or mead acid.

Clause 54. The composition of any one of clauses 42-53, wherein the cholesterol is synthetic cholesterol.

Clause 55. The composition of any one of clauses 42-54, wherein the methylated cyclodextrin is a methylated α-cyclodextrin, a methylated β-cyclodextrin, or a methylated γ-cyclodextrin.

Clause 56. The composition of any one of clauses 42-55, wherein the methylated cyclodextrin is methyl-β-cyclodextrin.

Clause 57. The composition of any one of clauses 42-56, further comprising an unmethylated cyclodextrin.

Clause 58. The composition of any one of clauses 42-57, wherein the molar ratio of the methylated cyclodextrin to the cholesterol is less than 10.5:1.

Clause 59. The composition of any one of clauses 42-58, wherein the molar ratio of the methylated cyclodextrin to other lipids in the composition is less than 11.5:1.

Clause 60. The composition of any one of clauses 42-59, which (i) comprises albumin; or (ii) does not comprise albumin.

Clause 61. The composition of any one of clauses 42-60, which (i) comprises a protein; or (ii) does not comprise a protein.

Clause 62. The composition of any one of clauses 42-61, further comprising 2-deoxy-D-glucose.

Clause 63. A method for culturing a T cell population, comprising incubating the population in a cell culture medium comprising a cyclodextrin and at least one lipid.

Clause 64. The method of clause 63, wherein the cell culture medium comprises the serum-free cell culture supplement composition of any one of clauses 45-62.

Clause 65. The method of clause 63 or 64, wherein the T cell population comprises CD8+ T cells.

Clause 66. The method of clause 63 or 64, wherein the T cell population comprises CD4+ T cells.

Clause 67. The method of clause 63 or 64, wherein the T cell population comprise CD8+ T cells and CD4+ T cells.

Clause 68. The method of any one of clauses 63-67, wherein the cell culture medium further comprises 2-deoxy-D-glucose.

Clause 69. A method of culturing a T cell population that comprises CD8+ T cells and CD4+ T cells while minimizing a change in the ratio of CD8+ T cells to CD4+ T cells within the population, the method comprising incubating the population in a cell culture medium comprising a cyclodextrin and a polyunsaturated fatty acid.

Clause 70. The method of clause 69, wherein the polyunsaturated fatty acid is an omega-6 polyunsaturated fatty acid.

Clause 71. The method of clause 70, wherein the omega-6 polyunsaturated fatty acid is linoleic acid.

Clause 72. The method of any one of clauses 69-71, wherein the cell culture medium further comprises cholesterol.

Clause 73. The method of any one of clauses 71 or 72, wherein the cell culture medium further comprises linolenic acid.

Clause 74. The method of clause 69, wherein the polyunsaturated fatty acid is linolenic acid.

Clause 75. The method of any one of clauses 69-74, wherein the cell culture medium further comprises arachidonic acid.

Clause 76. The method of any one of clauses 69-75, wherein minimizing a change in the ratio of CD8+ T cells to CD4+ T cells comprises maintaining a ratio of CD8+ T cells to CD4+ T cells in which the number of CD8+ T cells to CD4+ T cells differs by less than 25%, 20%, 15%, 10%, or 5% compared to the number of CD8+ T cells to CD4+ T cells when the population is first contacted with the medium.

Clause 77. The method of any one of clauses 69-76, wherein when the population is first contacted with the medium, then the population comprises a ratio of CD8+ T cells to CD4+ T cells of about 1:1.

Clause 78. The method of clause 69, wherein the medium comprises (i) a cyclodextrin; (ii) cholesterol; and (iii) fatty acids, wherein the fatty acids consist of linoleic acid, linolenic acid, and arachidonic acid.

Clause 79. The method of any one of clauses 69-78, wherein the medium lacks any one of, or any combination of, myristic acid, oleic acid, palmitic acid, palmitoleic acid, and stearic acid.

Clause 80. The method of any one of clauses 69-79, wherein the medium comprises a molar ratio of linoleic acid, linolenic acid, and/or arachidonic acid to other fatty acids of at least 1:1.

Clause 81. The method of any one of clauses 69-80, further comprising incubating the population for a sufficient period of time until the T cells have reached a desired number, stage of differentiation, and/or phenotype; and optionally harvesting T cells from the culture.

Clause 82. A method for preferentially expanding members of a T cell subpopulation, the method comprising exposing a mixed population of T cells to:
(i) cyclodextrin; and
(ii) fatty acids,
   wherein the molar ratio of two or more fatty acids is adjusted to induce the members of the T cell subpopulation to preferentially expand over members of other T cell subpopulations.

Clause 83. The method of clause 82, wherein the T cell subpopulation is CD8+ T cells.

Clause 84. The method of clause 83, wherein the mixed population of T cells is exposed to more polyunsaturated fatty acids than other fatty acids.

Clause 85. The method of clause 83, wherein the mixed population of T cells is exposed to more omega-6 polyunsaturated fatty acids than other fatty acids.

Clause 86. The method of any one of clauses 82-85, further comprising exposing the mixed population of T cells to 2-deoxy-D-glucose.

Clause 87. The method of clause 82, wherein the T cell subpopulation is CD4+ T cells.

Clause 88. The method of any one of clauses 82-87, wherein the T cells are primary T cells.

Clause 89. The method of any one of clauses 82-88, wherein the T cells have been isolated from the blood of a human subject.

Clause 90. The method of any one of clauses 82-87, wherein the T cells are genetically modified T cells.

Clause 91. The method of clause 90, wherein the T cells express a genetically modified T cell receptor.

Clause 92. The method of clause 90, wherein the T cells express a chimeric antigen receptor.

Clause 93. A method of culturing a T cell population that comprises CD8+ T cells and CD4+ T cells while increasing the ratio of CD8+ T cells to CD4+ T cells within the population, the method comprising incubating the population in a cell culture medium comprising 2-deoxy-D-glucose.

Clause 94. The method of clause 93, wherein the 2-deoxy-D-glucose is present at a level from about 0.1 mM to about 5 mM.

Clause 95. The method of clause 93 or 94, wherein the cell culture medium further comprises serum.

Clause 96. The method of clause 95, wherein the serum is human serum.

Clause 97. The method of clause 93 or 94, wherein the cell culture medium is a serum-free cell culture medium.

Clause 98. The method of any one of clauses 93-97, wherein the ratio of CD8+ T cells to CD4+ T cells in the population increases by at least 2-fold, 2.5-fold, 3-fold, or 3.5-fold within about 7 days after the population is first contacted with the medium.

Clause 99. The method of any one of clauses 93-98, wherein there are more CD4+ T cells than CD8+ T cells in the population when the population is first contacted with the medium.

Clause 100. The method of clause 99, wherein the ratio of CD4+ T cells to CD8+ T cells is at least 5:1 in the population when the population is first contacted with the medium.

Clause 101. The method of any one of clauses 63-100, wherein the T cells are primary T cells.

Clause 102. The method of any one of clauses 63-101, wherein the T cells have been isolated from the blood of a human subject.

Clause 103. The method of any one of clauses 63-100, wherein the T cells are genetically modified T cells.

Clause 104. The method of clause 103, wherein the T cells express a genetically modified T cell receptor.

Clause 105. The method of clause 103, wherein the T cells express a chimeric antigen receptor.

Clause 106. The method of any one of clauses 63-105, wherein the T cells are T regulatory cells (Tregs), T helper cells, Th17 cells, Th9 cells, T memory cells, T effector memory cells, T central memory cells, terminally differentiated effector (TTD) T cells, naïve T cells, or engineered T cells.

Clause 107. The method of any one of clauses 63-106, wherein the size of the T cell population doubles at least 3 times within 7 days.

Clause 108. The method of any one of clauses 63-107, wherein the size of the T cell population doubles at least 3, 4, or 5 times within 10 days.

Clause 109. The method of any one of clauses 63-108, wherein at least 75%, 80%, 85%, 90%, or 95% of the T cells in the T cell population are viable 7, 8, 9, or 10 days after the T cell population is first contacted with the medium.

Clause 110. The method of any one of clauses 63-109, wherein at least 95% of the T cells in the T cell population are viable 10 days after the T cell population is first contacted with the medium.

Clause 111. The method of any one of clauses 63-110, further comprising preparing the cultured T cells for administration to a subject suffering from or at risk of suffering from a disease or condition.

Clause 112. The method of clause 111, further comprising administering the T cells to the subject.

Clause 113. A method for treating a disease in a subject in need thereof, comprising administering to the subject T cells obtained by the method of any one of clauses 63-111.

Clause 114. The method of clause 113, wherein the disease is a hyperproliferative disorder.

Clause 115. The method of clause 113, wherein the disease is an autoimmune disease.

Clause 116. The method of clause 113, wherein the disease is an inflammatory disease.

Clause 117. The method of clause 113, wherein the disease is an allergic disease.

Clause 118. The method of clause 113, wherein the disease is an infectious disease.

Clause 119. The method of clause 118, wherein the infectious disease is a viral infection.

Clause 120. The method of clause 119, wherein the viral infection is a cytomegalovirus infection, a Epstein-Barr virus infection, or a human immunodeficiency virus infection.

Clause 121. The method of any one of clauses 113-120, wherein the subject has a suppressed immune system.

Clause 122. The method of any one of clauses 113-121, wherein the subject has received a tissue or organ transplant.

Clause 123. The method of any one of clauses 113-122, wherein the subject has acquired immune deficiency syndrome.

Clause 124. The method of any one of clauses 113-123, wherein the T cells are CD8+ T cells.

Clause 125. The method of any one of clauses 113-123, wherein the T cells are CD4+ T cells.

Clause 126. The method of any one of clauses 113-125, wherein the T cells are CD8+ T cells and CD4+ T cells.

Clause 127. A system for the supplementation of a T cell medium, comprising (i) a two or more different cyclodextrins, wherein each cyclodextrin is in a separate vessel; and (ii) two or more different fatty acids, wherein each fatty acid is in a separate vessel.

Clause 128. The system of clause 127, further comprising 2-deoxy-D-glucose.

Clause 129. A kit for culturing T cells comprising a serum free medium, a cyclodextrin, and one or more lipids.

Clause 130. The kit of clause 129, further comprising 2-deoxy-D-glucose.

Clause 131. The kit of clause 129 or 130, further comprising etomoxir.

Clause 132. A combination comprising (i) a population of T cells, (ii) a cell culture medium, and (ii) a supplement that comprises a cyclodextrin and at least one lipid.

Clause 133. A kit or combination comprising (i) a cell culture medium, and (ii) the composition of any one of clauses 42-62

Clause 134. A combination comprising (i) a population of diploid or non-diploid cells and (ii) a cell culture medium that comprises a cyclodextrin and at least one lipid.

Clause 135. The combination of clause 134, wherein the diploid cell produces a vaccine, a virus, a viral particle, a viral protein or nucleic acid, or a viral fragment under serum-free conditions.

Clause 136. A method for culturing a diploid cell population, comprising incubating the cell population in a cell culture medium comprising a cyclodextrin and at least one lipid.

Clause 137. The method of clause 136, wherein the cell culture medium is serum free.

Clause 138. The method of clause 137-137, wherein the cell population is selected from the group consisting of MRC-5, MRC-5 RCB, WI-38, 2BS, Walvax-2, IMR-90, IMR-91, KMB-17, and VERO cells.

Clause 139. The method of clause 137-138, wherein the cell produces a vaccine, a virus, a viral particle, a viral protein or nucleic acid, or a viral fragment under serum-free conditions.

Clause 140. A system for the supplementation of a diploid cell medium, comprising (i) a one or more different cyclodextrins, wherein each cyclodextrin is in a separate vessel; and (ii) two or more different fatty acids, wherein each fatty acid is in a separate vessel.

Clause 141. The system of clause 140, further comprising growth factors.

Clause 142. A system for the supplementation of a diploid cell medium, comprising (i) a cyclodextrins and (ii) two or more different fatty acids, wherein each fatty acid is in a separate vessel.

Clause 143. A kit for culturing a vaccine producing cell or cell line comprising a basal medium and a serum free growth supplement.

Clause 144. The kit for culturing the vaccine producing a cell or cell line of clause 143, wherein the serum free growth supplement further comprises a cyclodextrin, and one or more lipids.

Clause 145. The kit for culturing the vaccine producing cell or cell line of clause 144, wherein the vaccine producing cell is a diploid cell or a non-diploid cell.

Clause 146. The kit for culturing the vaccine producing cell or cell line of clause 145, wherein the diploid cell is a human cell.

Clause 147. A serum-free cell culture medium composition comprising linoleic acid, at least one other omega-6 fatty acid, cholesterol, and a cyclodextrin.

Clause 148. A serum-free cell culture supplement composition, comprising linoleic acid, at least one other omega-6 fatty acid, cholesterol, and a cyclodextrin.

Clause 149. The serum-free cell culture medium composition of clause 147, or the serum-free cell culture supplement composition of clause 148, wherein the cyclodextrin is a methylated cyclodextrin.

Clause 150. The serum-free cell culture medium composition, or the serum-free cell culture supplement composition of clause 149, wherein the methylated cyclodextrin is present at a level from about 50 μM to about 200 μM.

Clause 151. The serum-free cell culture medium composition of any of clauses 147 to 150, or the serum-free cell culture supplement composition of any of clauses 148 to 150, wherein the cholesterol is a synthetic cholesterol; and/or, wherein the cholesterol is present at a level from about 5 μM to about 30 μM.

Clause 152. The serum-free cell culture medium composition of any of clauses 147 to 151, or the serum-free cell culture supplement composition any of clauses 148 to 151, wherein the at least one other omega-6 fatty acid is a polyunsaturated omega-6 fatty acid.

Clause 153. The serum-free cell culture medium composition of any of clauses 147 to 152, or the serum-free cell culture supplement composition of any of clauses 148 to 152, wherein the at least one other omega-6 fatty acid is arachidonic acid.

Clause 154. The serum-free cell culture medium composition of any of clauses 147 to 153, or the serum-free cell culture supplement composition of any of clauses 148 to 153, wherein the polyunsaturated omega-6 fatty acid is selected from the group consisting of arachidonic acid, linoleic acid, linolenic acid, myristic acid, oleic acid, palmitic acid, palmitoleic acid and stearic acid.

Clause 155. The serum-free cell culture supplement composition of any of clauses 148 to 154, wherein the effective dilution of the supplement is from about 1:10 to about 1:5000.

Clause 156. The serum-free cell culture medium composition of clauses 147, or the serum-free cell culture supplement composition of clause 148, that is capable of culturing a cell that can produce a vaccine, a virus, a viral particle, a viral protein or nucleic acid, or a viral fragment.

Clause 157. The serum-free cell culture medium composition or the serum-free cell culture supplement composition of any of clauses 147 to 156, wherein the cell is an animal cell.

Clause 158. The serum-free cell culture medium composition or the serum-free cell culture supplement composition of any of clauses 147 to 157, wherein the animal cell is a bovine cell, a canine cell, a feline cell, an insect cell, an avian cell, a primate cell or a human cell.

Clause 159. The serum-free cell culture medium composition or the serum-free cell culture supplement composition of any of clauses 147 to 158, wherein the animal cell is a diploid cell.

Clause 160. The serum-free cell culture medium composition or the serum-free cell culture supplement composition of any of clauses 147 to 159, wherein the cell is selected from the group consisting of MRC-5, MRC-5 RCB, MRC-9, WI-38, 2BS, Walvax-2, IMR-90, IMR-91, KMB-17, HUT series cell, Chang liver, U937, MDCK, CD4-expressing T cell, CD8-expressing T cell, VERO and any clone of the preceding cells.

Clause 161. The serum-free cell culture medium composition, or the serum-free cell culture supplement composition of any of clauses 147 to 160, wherein the medium or supplement increases: the growth of the cell, the viable cell density of the cell, the viral titer of a virus infected cell, or a combination thereof.

Clause 162. The serum-free cell culture medium composition or the serum-free cell culture supplement composition of any of clauses 147 to 161, wherein said cell is infected with a virus.

Clause 163. The serum-free cell culture medium composition or the serum-free cell culture supplement composition of any of clauses 147 to 162, wherein the virus is an animal virus, a plant virus or a bacteriophage.

Clause 164. The serum-free cell culture medium composition or the serum-free cell culture supplement composition of any of clauses 147 to 163, wherein the virus is selected from the group consisting of Varicella zoster virus (VZV), Rubella, Measles, Mumps, Hepatitis A, Adenovirus, Poliomyelitis, Rotavirus, Smallpox, Chickenpox, Yellow fever, Papillomavirus, Ebola virus, HIV, Rabies or vesicular stomatitis virus (VSV), and Dengue virus.

Clause 165. The serum-free cell culture medium composition or the serum-free cell culture supplement composition of any of clauses 147 to 164, wherein the viral particle is derived from a Parvoviridae family, Retroviridae family, Flaviviridae family or a bacteriophage.

Clause 166. The serum-free cell culture supplement composition of clause 148 that is added to a basal medium to culture a diploid cell capable of producing a vaccine, a virus, a viral particle, a viral protein or nucleic acid, or a viral fragment, wherein the cell is cultured under serum-free conditions.

Clause 167. A method for culturing a diploid cell population, comprising incubating the cell population in a cell culture medium comprising a cyclodextrin and at least one lipid.

Clause 168. The method of culturing a cell population of clause 167 that comprises vaccine producing diploid cells, the method comprising incubating the cell population in a serum-free, cell culture medium comprising:
(i) a cyclodextrin, linoleic acid, at least one other omega-6 fatty acid and cholesterol, or,
(ii) a suitable dilution of the supplements described in Table 1 and/or Table 2; wherein the culture increases viable cell density in said serum-free, cell culture medium compared to a viable cell density in a serum-containing medium without cyclodextrin.

Clause 169. The method for culturing a diploid cell population of clause 167, wherein the cyclodextrin is a methylated cyclodextrin.

Clause 170. The method for culturing a diploid cell population of clause 169, wherein the methylated cyclodextrin is present at a level from about 50 μM to about 200 μM.

Clause 171. The method for culturing a diploid cell population of clauses 168 or 169, wherein the at least one other omega-6 fatty acid is a polyunsaturated omega-6 fatty acid.

Clause 172. The method for culturing a diploid cell population of clauses 168 or 169, wherein the polyunsaturated omega-6 fatty acid is selected from the group consisting of arachidonic acid, linolenic acid, myristic acid, oleic acid, palmitic acid, palmitoleic acid and stearic acid.

Clause 173. The method for culturing a diploid cell population of any of clauses 167 to 172, wherein:
(i) the medium or supplement increases: the growth of the cell, the viable cell density of the cell, the viral titer of a virus infected cell, or a combination thereof, and/or,
(ii) the diploid cell is capable of producing a vaccine, a virus, a viral particle, a viral protein or nucleic acid, or a viral fragment thereof under serum-free conditions; and/or,
(iii) said cell is infected with a virus.

Clause 174. The method for culturing a diploid cell population of any of clauses 167 to 173, wherein:
(i) the virus is an animal virus, a plant virus or a bacteriophage; and/or,
(ii) the virus is selected from the group consisting of Varicella zoster virus (VZV), Rubella, Measles, Mumps, Hepatitis A, Adenovirus, Poliomyelitis, Rotavirus, Smallpox, Chickenpox, Yellow fever, Papillomavirus, Ebola virus, HIV, Rabies or vesicular stomatitis virus (VSV), and Dengue virus; and/or,
(iii) the viral particle is derived from a Parvoviridae family, Retroviridae family, Flaviviridae family or a bacteriophage.

Clause 175. The method of any of clauses 167 to 174, wherein the cell population is selected from the group consisting of MRC-5, MRC-5 RCB, MRC-9, WI-38, 2BS, Walvax-2, IMR-90, IMR-91, KMB-17, HUT series cell, Chang liver, U937, MDCK, CD4-expressing T cell, CD8-expressing T cell, VERO and any clone of the preceding cells.

Clause 176. A combination comprising:
(i) a population of cells; (ii) a serum-free cell culture medium that comprises a cyclodextrin and at least one lipid, or,
(i) a population of cells; (ii) a serum-free basal cell culture medium; and (iii) a supplement that comprises a cyclodextrin and at least one lipid, or
(i) a population of cells; (ii) a serum-free basal cell culture medium; and (iii) a suitable dilution of the supplements described in Table 1 and/or Table 2.

Clause 177. The combination of clause 176, wherein:
(i) the cell is animal cell; or,
(ii) the animal cell is a bovine cell, a feline cell, an insect cell, an avian cell, a primate cell or a human cell; or,
(iii) the animal cell is a diploid cell; or,
(iv) the cell is selected from the group consisting of MRC-5, MRC-5 RCB, MRC-9, WI-38, 2BS, Walvax-2, IMR-90, IMR-91, KMB-17, HUT series cell, Chang liver, U937, MDCK, CD4-expressing T cell, CD8-expressing T cell, VERO and any clone of the preceding cells.

Clause 178. The combination of clauses 176 to 177, wherein the diploid cell produces a vaccine, a virus, a viral particle, a viral protein or nucleic acid, or a viral fragment under serum-free conditions.

Clause 179. A method of making a serum-free diploid cell culture medium, comprising admixing (i) a basal medium; and either (ii) a supplement that comprises a cyclodextrin and at least one lipid; or (ii) a suitable dilution of the supplements described in Table 1 and/or Table 2.

Clause 180. The method of making a serum-free diploid cell culture medium of clause 179, wherein the supplement further comprises growth factors.

Clause 181. A system for the supplementation of a diploid cell medium, comprising (i) a one or more different cyclodextrins, wherein each cyclodextrin is in a separate vessel; and (ii) two or more different fatty acids, wherein each fatty acid is in a separate vessel.

Clause 182. A kit for culturing a cell or cell line comprising: (i) a population of cells; (ii) a serum-free cell culture medium that comprises a cyclodextrin and at least one lipid, or,
(i) a population of cells; (ii) a serum-free basal cell culture medium; and (iii) a supplement that comprises a cyclodextrin and at least one lipid, or,
(i) a population of cells; (ii) a serum-free basal cell culture medium; and (iii) a suitable dilution of the supplements described in Table 1 and/or Table 2.

Clause 183. The kit of clause 182, wherein:
(i) the cell is animal cell; or,
(ii) the animal cell is a bovine cell, a canine cell, a feline cell, an insect cell, an avian cell, a primate cell or a human cell; or,
(iii) the animal cell is a diploid cell; or,
(iv) the cell is selected from the group consisting of MRC-5, MRC-5 RCB, MRC-9, WI-38, 2BS, Walvax-2, IMR-90, IMR-91, KMB-17, HUT series cell, Chang liver, U937, MDCK, CD4-expressing T cell, CD8-expressing T cell, VERO and any clone of the preceding cells.

Clause 184. The kit of clauses 182 to 183, wherein the cell produces a vaccine, a virus, a viral particle, a viral protein or nucleic acid, or a viral fragment under serum-free conditions.

1.-39. (canceled)

40. A combination comprising (i) a population of T cells and (ii) a culture medium that comprises a cyclodextrin and at least one lipid.

41.-50. (canceled)

51. The combination of claim 40, wherein the cyclodextrin is methyl-β-cyclodextrin.

52.-56. (canceled)

57. The combination of claim 40, further comprising 2-deoxy-D-glucose.

58. A method for culturing a T cell population, the method comprising incubating the population in a cell culture medium comprising a cyclodextrin and at least one lipid.

59. (canceled)

60. The method of claim 58, wherein the T cell population comprises CD8+ T cells.

61. The method of claim 58, wherein the T cell population comprises CD4+ T cells.

62. (canceled)

63. The method of claim 58, wherein the cell culture medium further comprises 2-deoxy-D-glucose.

64. The method of claim 58 wherein the T cell population comprises CD8+ T cells and CD4+ T cells, the method further comprising incubating the T cell population in the cell culture medium,
wherein the change in the ratio of CD4+ T cells to CD8+ T cells within the population is less than 50% after 10 days of culture, and
wherein the cell culture medium contains between 0.1 mM to about 10 mM 2-deoxy-D-glucose.

65.-67. (canceled)

68. The method of claim 64, wherein when the population is first contacted with the medium, then the population comprises a ratio of CD4+ T cells to CD8+ T cells of about 1:1.

69. The method of claim 58 comprising preferentially expanding members of a T cell subpopulation present in the T cell population, the method further comprising exposing the T cell population to:
(i) cyclodextrin; and
(ii) two or more fatty acids,
wherein the molar ratio of two or more fatty acids is adjusted to induce the members of the T cell subpopulation to preferentially expand over members of other T cell subpopulations.

70. The method of claim 69, wherein the T cell subpopulation is CD8+ T cells.

71. The method of claim 69, wherein the T cell subpopulation is CD4+ T cells.

72. The method of claim 69, further comprising exposing the mixed population of T cells to 2-deoxy-D-glucose.

73. (canceled)

74. The method of claim 69, wherein the T cells are primary T cells.

75. (canceled)

76. The method of claim 69, wherein the T cells are genetically modified T cells.

77. The method of claim 76, wherein the T cells express a genetically modified T cell receptor.

78. The method of claim 69, wherein the T cells express a chimeric antigen receptor.

79. A method of culturing a T cell population that comprises CD8+ T cells and CD4+ T cells while increasing the ratio of CD8+ T cells to CD4+ T cells within the population, the method comprising incubating the population in a cell culture medium comprising 2-deoxy-D-glucose.

80.-82. (canceled)

83. The method of claim 79, wherein the ratio of CD8+ T cells to CD4+ T cells in the population increases by from 2-fold to 5-fold after 7 days after first contacted with the culture medium.

84.-94. (canceled)
